(19) United States
(12) Patent Application Publication
(10) Pub. No.: US 2010/0144751 A1
(43) Pub. Date: Jun. 10, 2010

(54) IMIDAZO[1,2-a]PYRIDINE COMPOUNDS AS RECEPTOR TYROSINE KINASE INHIBITORS

(75) Inventors: Fredrik P. Marmsater, Boulder, CO (US); Mark C. Munson, Louisville, CO (US); James P. Rizzi, Longmont, CO (US); John E. Robinson, Commerce City, CO (US); Stephen T. Schlacter, Boulder, CO (US); George T. Topalov, Superior, CO (US); Joseph P. Lyssikatos, Piedmont, CA (US)

Correspondence Address: VIKSNINS HARRIS & PADYS PLLP, P.O. BOX 111098, ST. PAUL, MN, 55111-1098, US

(73) Assignee: ARRAY BIOPHARMA INC., Boulder, CO (US)

(21) Appl. No.: 12/593,041

(22) PCT Filed: Mar. 27, 2008
(86) PCT No.: PCT/US08/58385

§ 371 Date: Jan. 5, 2010

Related U.S. Application Data
(60) Provisional application No. 60/908,556, filed on Mar. 28, 2007.

(51) Int. Cl. A61K 31/496; C07D 471/04; A61K 31/4709; A61P 35/00

(52) U.S. Cl. 514/253.06; 544/363; 546/121; 514/300

(57) ABSTRACT

Compounds of Formula (I): are receptor tyrosine inhibitors useful in the treatment of diseases mediated by class (3) and class (5) receptor tyrosine kinases. Particular compounds of this invention have also been found to be inhibitors of Pim-1.

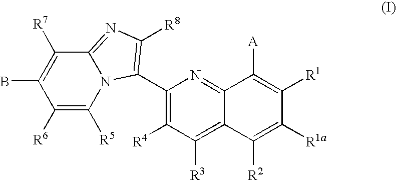

The present invention relates to novel compounds, to pharmaceutical compositions comprising the compounds, to a process for making the compounds and to the use of the compounds in therapy. More particularly, it relates to certain imidazopyridine compounds useful in the treatment and prevention of diseases mediated by class 3 and class 5 receptor tyrosine kinases. Particular compounds of this invention have also been found to be inhibitors of Pim-1.

Receptor tyrosine kinases (RTK's) include the class 3 receptor tyrosine kinases (PDGF-α, PDGFR-β, MCSF-1R, c-kit, and FLT3) and the class 5 receptor tyrosine kinases (VEGFR and KDR). It is known that such kinases are frequently aberrantly expressed in common human cancers, such as breast cancer, gastrointestinal cancer such as colon, rectal or stomach cancer, leukemia, and ovarian, bronchial or pancreatic cancer, renal cell cancer and gliomas.

FLT3 (fins-like tyrosine kinase; also known as Flk-2) is a member of the class 3 receptor tyrosine kinase (RTK) family, and is presumed to be involved in the hematopoietic system (Rosnet, et al., 1991, Genomics 9:380-385, Rosnet, et al., 1993, Blood 82:1110-1119). Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the FLT3 receptor have been found in about 35% of patients with acute myeloblastic leukemia (AML), and are associated with a poor prognosis. These types of mutations are associated with constitutive activation of the tyrosine kinase activity of FLT3, and result in proliferation and viability signals in the absence of ligand. Patients expressing the mutant form of the receptor have been shown to have a decreased chance for cure. In addition to activating mutations, ligand dependent (autocrine or paracrine) stimulation of over-expressed wild-type FLT3 contributes to AML. Thus, there is accumulating evidence for a role for hyper-activated (mutated) FLT3 kinase activity in human leukemias and myelodysplastic syndrome. FLT3 inhibitors may also be useful for treating immune related disorders, diseases of the bone, and inflammation based on the role of FLT3 in dendritic cells.

PDGFR is expressed on early stem cells, mast cells, myeloid cells, mesenchymal cells, and smooth muscles cells and is involved in the process of angiogenesis through its expression in pericytes. PDGFR-β has been implicated in myeloid leukemias. Recently, it was shown that activating mutations in PDGFR-α kinase domain are in gastrointestinal stromal tumors (GIST) (Wong et al., 2007, Histopathology 51(6): 758-762).

In addition, blockade of PDGF signaling has been shown to reduce the development of fibrosis in various experimental models (Yoshiji et al., 2006, International Journal Molecular Medicine 17: 899-904).

Accordingly, it has been recognized that inhibitors of receptor tyrosine kinases are useful as inhibitors of the growth of mammalian cancer cells or for treating immune related disorders.

The Pim kinases are a family of three distinct vertebrate protein serine/threonine kinases (Pim-1, -2 and -3) belonging to the calmodulin-dependent protein kinase-related (CAMK) group. The over-expression of Pim-1 has been reported in various human lymphomas and acute leukemias (Amson, R. et al, Proc. Natl. Acad. Sci. U.S.A., 1989, 86: 8857-8861). In addition, there is evidence that Pim-1 is over-expressed in prostatic neoplasia and human prostate cancer (Valdman, A. et al, The Prostate, 2004, 60: 367-371; Cibull, T. L. et al, J. Clin. Pathol., 2006, 59: 285-288) and may serve as a useful biomarker in identification of prostate cancer (Dhanasekaran, S. M. et al, Nature, 2001, 412(13): 822-826). Recently, it has been discovered that Pim-1 is up-regulated by Flt-3 and may play an ancillary role in Flt-3 mediated cell survival (Kim, K. T. et al Neoplasia, 2005, 105(4): 1759-1767). Since Flt-3 itself is implicated in leukemias like AML, additional knockdown of Pim-1 may be a useful approach to treating leukemias driven by Flt-3 or various mutations. Accordingly, Pim-1 inhibitors may be useful as therapeutic agents for a variety of cancers such as hematological cancers.

Tyrosine kinase inhibitors are known in the art. U.S. Pat. No. 7,125,888 describes certain imidazo[1,2-a]pyridine compounds substituted at the 3 position with a pyridyl, thiazolyl, oxazolyl or phenyl group and at the 7 position with an optionally substituted phenyl or pyridone group, which are purported to be tyrosine kinase inhibitors. U.S. patent publication 2005/0124637 discloses certain purine derivatives as inhibitors of receptor tyrosine kinases, including FLT3. PCT publication number WO 01/40217 and U.S. Pat. No. 7,019,147 disclose certain benzimidazole compounds having activity as tyrosine kinase inhibitors.

It has now been found that certain imidazo[1,2-a]pyridine compounds bearing a quinolinyl group at the 3 position of the imidazopyridine ring are inhibitors of receptor tyrosine kinases, in particular class 3 and class 5 receptor tyrosine kinases, which are useful for treating diseases mediated by class 3 and class 5 receptor tyrosine kinases, such as cancers, fibrosis, sclerosis, autoimmune disorders and scleroderma.

In certain embodiments, the imidazopyridine compounds are class 3 receptor tyrosine kinases inhibitors. In particular embodiments, the compounds are inhibitors of the class 3 receptor tyrosine kinases PDGFR and FLT3.

A subset of compounds of the imidazopyridine compounds disclosed herein has also been found to inhibit the kinase PIM-1.

Accordingly, provided is a compound of general Formula I

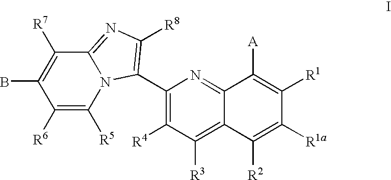

or a pharmaceutically acceptable salt thereof, wherein:

A is a 5-8 membered N-linked heterocyclic ring having at least one nitrogen atom and optionally substituted with one or more R⁹ groups;

B is H, CN, OR^{h}, Ar¹, hetAr², C(O)NRⁱR^{j}, C(O)-hetCyc³, CO₂(1-6 C alkyl), C(O)NH(1-6C alkyl)-hetCyc³, C(O)(1-6C alkyl)-hetCyc³, SR^{k}, SO₂N(1-6C alkyl)₂, or (1-6C alkyl)NR′R″;

R¹, R², R³ and R⁴ are independently H, F, Cl, CN, Me, Et, isopropyl, cyclopropyl, C(O)NR′R″, CH₂OH, or hetAr³;

R^{1a} is H, F, Cl, CN, Me, or CF₃;

R⁵, R⁶, R⁷ and R⁸ are independently H, F, Cl, CN or Me;

each R⁹ is independently selected from halogen, CN, CF₃, (1-6C)alkyl, NR^{a}R^{b}, -(1-6C alkyl)NR^{a}R^{c}, OR^{a}, (1-6C alkyl)OR^{a} [optionally substituted with amino], C(O)NR^{a}R^{c}, C(O)(CR^{x}R^{y})NR^{a}R^{c}, NHC(O)R^{e}, NHC(O)(CR^{m}Rⁿ)NR^{a}R^{c}, NHC(O)NR^{f}R^{g}, (1-6C alkyl)-hetAr¹, (1-6C alkyl)-hetCyc¹, oxo and CO₂(1-6C alkyl);

each R^{a} is independently H or (1-6C)alkyl;

each R^{b} is independently H, (1-6C)alkyl, (1-6C alkyl)OH, (3-6C)cycloalkyl, CH₂hetAr⁴, (1-6C fluoroalkyl) or -(1-6C alkyl)-O-(1-6C alkyl),

or NR^{a}R^{b} forms a 4-6 membered heterocyclic ring optionally substituted with OH;

each R^{c} is independently H, (1-6C)alkyl, (3-6C)cycloalkyl, or aryl;

each R^{e} is independently (1-6C alkyl);

each R^{f} and R^{g} is independently H or (1-6C alkyl);

R^{h} is H, CF₃, (1-6C)alkyl, (1-6Calkyl)-(3-6C cycloalkyl), (1-6C alkyl)-O-(1-6C alkyl), (1-6C alkyl)OH, (1-6C alkyl)-S-(1-6C alkyl), (1-6C alkyl)NR′R″, hetCyc⁴, (1-6C alkyl)hetCyc⁴, (1-6C alkyl)aryl, or (1-6C alkyl)-hetAr⁵;

Rⁱ is H or 1-6C alkyl;

R^{j} is (1-6C)alkyl, (1-6C alkyl)-O-(1-6C alkyl), or (1-6C alkyl)-OH;

R^{k} is (1-6C)alkyl, (3-6C)cycloalkyl, or (1-6C alkyl)-O-(1-6C alkyl);

R^{m} and Rⁿ are independently H or (1-6C alkyl);

R^{x} and R^{y} are independently H or (1-6C alkyl),

or R^{x} and R^{y} together with the atom to which they are attached form a cyclopropyl ring;

Ar¹ is aryl optionally substituted with OH, O-(1-6C alkyl), C(O)₂(1-6C alkyl), or (1-6C alkyl)NR′R″;

hetCyc¹ is a 5-6 membered heterocyclic ring which is optionally substituted with (1-6C)alkyl or OH;

hetCyc³ and hetCyc⁴ are independently a 5 or 6 membered heterocyclic ring optionally substituted with OH or —O(1-6C alkyl);

hetAr¹ and hetAr² are independently a 5-6 membered heteroaryl ring optionally substituted with one to three groups independently selected from (1-6C)alkyl, (3-6C)cycloalkyl, halogen, CN, CF₃, OCH₂F, OCF₃, O(1-6C alkyl), O(3-6C)cycloalkyl, and NR′R″;

hetAr³ and hetAr⁴ are independently a 5-6 membered heteroaryl ring;

hetAr⁵ is a 5-6 membered heteroaryl ring optionally substituted with (1-6C)alkyl; and

R′ and R″ are independently H or (1-6C)alkyl.

Compounds of Formula I include compounds having the general formula Ia:

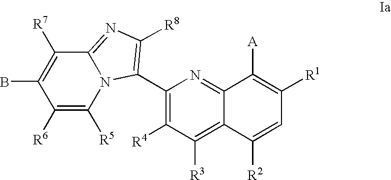

wherein:

A is a 5-8 membered N-linked heterocyclic ring having at least one nitrogen atom and optionally substituted with one or more R⁹ groups;

B is H, CN, OR^{h}, Ar¹, hetAr², C(O)NRⁱR^{j}, C(O)-hetCyc³, C(O)(1-6C alkyl)-hetCyc³, SR^{k}, SO₂N(1-6C alkyl)₂, or (1-6C alkyl)NR′R″;

R¹, R², R³ and R⁴ are independently H, F, Cl, CN, Me, C(O)NR′R″, CH₂OH, or hetAr³;

R⁵, R⁶, R⁷ and R⁸ are independently H, F, Cl, CN or Me;

each R⁹ is independently selected from halogen, CN, CF₃, (1-6C)alkyl, NR^{a}R^{b}, -(1-6C alkyl)NR^{a}R^{c}, OR^{a}, (1-6C alkyl)OR^{a} [optionally substituted with amino], C(O)NR^{a}R^{c}, C(O)(CR^{x}R^{y})NR^{a}R^{c}, NHC(O)R^{e}, NHC(O)(CR^{m}Rⁿ)NR^{a}R^{c}, NHC(O)NR^{f}R^{g}, (1-6C alkyl)-hetAr¹, (1-6C alkyl)-hetCyc¹ and oxo;

R^{a} is H or (1-6C)alkyl;

R^{b} is H, (1-6C)alkyl, (1-6C alkyl)OH, (3-6C)cycloalkyl, or CH₂hetAr⁴;

R^{c} is H, (1-6C)alkyl, (3-6C)cycloalkyl, or aryl;

R^{e} is (1-6C alkyl);

R^{f} and R^{g} are independently H or (1-6C alkyl);

R^{h} is H, CF₃, (1-6C)alkyl, (1-6Calkyl)-(3-6C cycloalkyl), (1-6C alkyl)-O-(1-6C alkyl), (1-6C alkyl)OH, (1-6C alkyl)-S-(1-6C alkyl), (1-6C alkyl)NR′R″, hetCyc⁴, (1-6C alkyl)hetCyc⁴, (1-6C alkyl)aryl, or (1-6C alkyl)-hetAr⁵;

Rⁱ is H or 1-6C alkyl;

R^{j} is (1-6C)alkyl, (1-6C alkyl)-O-(1-6C alkyl), or (1-6C alkyl)-OH;

R^{k} is (1-6C)alkyl, (3-6C)cycloalkyl, or (1-6C alkyl)-O-(1-6C alkyl);

R^{m} and Rⁿ are independently H or (1-6C alkyl);

R^{x} and R^{y} are independently H or (1-6C alkyl),

or R^{x} and R^{y} together with the atom to which they are attached form a cyclopropyl ring;

Ar¹ is aryl optionally substituted with OH, O-(1-6C alkyl), C(O)₂(1-6C alkyl), or (1-6C allyl)NR′R″;

hetCyc¹ is a 5-6 membered heterocyclic ring which is optionally substituted with (1-6C)alkyl or OH;

hetCyc³ and hetCyc⁴ are independently a 5 or 6 membered heterocyclic ring optionally substituted with OH;

hetAr¹ and hetAr² are independently a 5-6 membered heteroaryl ring optionally substituted with one to three groups independently selected from (1-6C)alkyl, (3-6C)cycloalkyl, halogen, CN, CF₃, OCH₂F, OCF₃, O(1-6C alkyl), O(3-6C)cycloalkyl, and NR′R′;

hetAr³ and hetAr⁴ are independently a 5-6 membered heteroaryl ring;

hetAr⁵ is a 5-6 membered heteroaryl ring optionally substituted with (1-6C)alkyl; and

R′ and R″ are independently H or (1-6C)alkyl.

In certain embodiments of Formula I, R¹ is H, F, Cl, Me, Et or isopropyl.

In certain embodiments of Formula I, R¹ is H, F or Cl.

In certain embodiments of Formula I, R¹ is H, Me, Et or isopropyl.

In certain embodiments of Formula I, R¹ is H.

In certain embodiments of Formula I, R^{1a} is H, F, Cl or Me.

In certain embodiments of Formula I, R^{1a} is H, F, or CF₃.

In certain embodiments of Formula I, R^{1a} is H or F.

In certain embodiments of Formula I, R^{1a} is H.

In certain embodiments of Formula I, R^{1a} is F.

In certain embodiments of Formula I, R² is H, F, Cl, Me, Et or isopropyl.

In certain embodiments of Formula I, R² is H, F or Cl.

In certain embodiments of Formula I, R² is H, Me, Et or isopropyl.

In certain embodiments of Formula I, R² is H.

In certain embodiments of Formula I, R² is F.

In certain embodiments of Formula I, R³ is H methyl, ethyl, isopropyl, cyclopropyl, or hetAr³. Examples of hetAr³ include 5 membered heteroaryl rings having a nitrogen atom and optionally having a second heteroatom selected from N and O. An example is oxazolyl. A particular value for R³ is the structure:

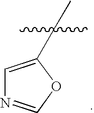

In certain embodiments of Formula I, R³ is H methyl, ethyl, isopropyl, cyclopropyl or oxazolyl.

In certain embodiments of Formula I, R³ is H, methyl, ethyl, isopropyl or cyclopropyl.

In certain embodiments of Formula I, R³ is H, methyl, or hetAr³.

In certain embodiments of Formula I, R³ is H, methyl, or oxazolyl.

In certain embodiments of Formula I, R³ is H.

In certain embodiments of Formula I, R³ is methyl.

In certain embodiments of Formula I, R³ is hetAr³. In certain embodiments, R³ is oxazolyl.

In certain embodiments of Formula I, R⁴ is H, F, Cl, Me, Et or isopropyl.

In certain embodiments of Formula I, R⁴ is H, F or Me.

In certain embodiments of Formula I, R⁴ is H, F or Cl.

In certain embodiments of Formula I, R⁴ is H.

In certain embodiments, R⁴ is F.

In certain embodiments, R⁵, R⁶, R⁷ and R⁸ are independently selected from H, F and Me.

In certain embodiments of Formula I, R⁵ is H.

In certain embodiments of Formula I, R⁶ is H.

In certain embodiments of Formula I, R⁷ is H.

In certain embodiments of Formula I, R⁸ is H.

In certain embodiments, of Formula I, each of R¹ and R⁴ is hydrogen.

In certain embodiments, of Formula I, each of R⁵, R⁶, R⁷ and R⁸ is hydrogen.

In certain embodiments, of Formula I, each of R¹, R⁴, R⁵, R⁶, R⁷ and R⁸ is hydrogen.

In certain embodiments, of Formula I, each of R¹, R^{1a}, R², R³, R⁴, R⁵, R⁶, R⁷ and R⁸ is hydrogen.

In certain embodiments of Formula I, A is a 5-8 membered heterocyclic ring having one or two nitrogen atoms. Particular values for A include piperidinyl, piperazinyl and pyrrolidinyl rings, which may be unsubstituted or substituted with one or more R⁹ groups.

In certain embodiments, A is substituted with one or more R⁹ groups independently selected from halogen, (1-6C)alkyl, NR^{a}R^{b}, -(1-6C alkyl)NR^{a}R^{c}, OR^{a}, (1-6C alkyl)OR^{a} [optionally substituted with amino], C(O)NR^{a}R^{c}, C(O)(CR^{x}R^{y})NR^{a}R^{c}, NHC(O)R^{e}, NHC(O)(CR^{m}Rⁿ)NR^{a}R^{c}, NHC(O)NR^{f}R^{g}, (1-6C alkyl)-hetAr¹, (1-6C alkyl)-hetCyc¹, and oxo.

Examples of R⁹ groups having the formula (1-6C)alkyl include methyl, ethyl, and propyl.

Examples of R⁹ groups having the formula NR^{a}R^{b} include groups where R^{a} is H or Me and R^{b} is H, methyl, ethyl, propyl, butyl, t-butyl, CH₂C(CH₃)₂OH, cyclopropyl, phenyl, or CH₂hetAr⁴. Particular examples of hetAr⁴ include 6 membered heteroaryl rings having 1-2 nitrogen atoms, for example pyridyl and pyrimidyl. Particular values of R⁹ when represented by NR^{a}R^{b} include NH₂ and NMe₂.

In other embodiments, R⁹ is a group having the formula NR^{a}R^{b} wherein R^{a} is H or (1-6C alkyl), and R^{b} is H, (1-6C alkyl), (1-6C fluoroalkyl), (1-6C alkyl)-O-(1-6C alkyl) or (1-6C alkyl)OH. In a particular embodiment, R⁹ is selected from NH₂, NHMe, NMe₂, NHCH(CH₃)CH₂F, NHCH₂CH₂OMe, NHCH₂CH₂OH and N(CH₃)CH₂CH₂OH.

In other embodiments, R⁹ is a group having the formula NR^{a}R^{b} where NR^{a}R^{b} forms a 4-6 membered heterocyclic ring optionally substituted with OH. Examples of heterocyclic rings include azetidinyl, pyrrolidinyl and piperidinyl rings. In certain embodiments, NR^{a}R^{b} is an azetidinyl ring optionally substituted with OH. In particular embodiments, NR^{a}R^{b} is 1-azedidin-3-ol.

Examples of R⁹ groups having the formula (I-6C alkyl)NR^{a}R^{c} include groups where R^{a} is H or Me and R^{c} is H, methyl, or cyclopropyl. Particular values of R⁹ when represented by (1-6C alkyl)NR^{a}R^{c} include CH₂NH₂ and CH₂CH₂NMe₂.

Examples of R⁹ groups having the formula OR^{a} include groups where R^{a} is H or methyl. Particular mention is made of OH.

Examples of R⁹ groups having the formula (1-6C alkyl)OR^{a} optionally substituted with an amino group include groups where R^{a} is H. Particular values of such substituents include CH₂OH. A further example of R⁹ is CH(NH₂)CH₂OH.

Examples of R⁹ groups having the formula C(O)NR^{a}R^{c}include groups where R^{a} is H or Me and R^{c} is (1-6C)alkyl, for example methyl. A particular value of R⁹ is C(O)NHMe.

Examples of R⁹ groups having the formula C(O)(CR^{x}R^{y})NR^{a}R^{c} include groups wherein R^{x} and R^{y} are independently H or methyl, R^{a} is H or methyl, and R^{c} is H or (1-6C)alkyl, for example methyl. In another embodiment, R^{x} and R^{y} together with the atom to which they are attached form a cyclopropyl ring. That is, CR^{x}R^{y} forms a cyclopropyl ring. Particular values of R⁹ include C(O)C(CH₃)₂NH₂, C(O)CH(CH₃)NH₂, C(O)CH₂NH₂, C(O)CH₂NMe₂, and C(O)C(cyclopropylidine)NH₂.

Examples of R⁹ groups having the formula NHC(O)R^{e} include groups wherein R^{e} is methyl.

Examples of R⁹ groups having the formula NHC(O)(CR^{m}Rⁿ)NR^{a}R^{c} include groups wherein R^{m} and Rⁿ are independently H or methyl, R^{a} is H or Me, and R^{c} is H or Me. Particular values of R⁹ include NHC(O)CH₂NH₂, NHC(O)CH(CH₃)NH₂, and NHC(O)C(CH₃)₂NH₂.

Examples of R⁹ groups having the formula NHC(O)NR^{f}R^{g} include groups wherein R^{f} and R^{g} are independently H or Me. A particular value includes NHC(O)NH₂.

Examples of R⁹ groups having the formula (1-6C alkyl)-hetAr¹ include groups wherein hetAr¹ is a 6 membered heteroaryl having at least one nitrogen atom, for example a pyridyl group. Particular values of R⁹ include CH₂(pyrid-2-yl) and CH₂(pyrid-4-yl).

Examples of R⁹ groups having the formula (I-6C alkyl)-hetCyc¹ include groups wherein hetCyc¹ is a 5-6 membered ring having 1-2 nitrogen atoms. Particular values of hetCyc¹ include optionally substituted piperazinyl or pyrrolidinyl rings. In certain embodiments, hetCyc¹ is optionally substituted with OH or an alkyl group, for example methyl. Particular values of R⁹ include CH₂-(4-methylpiperazinyl) and CH₂(3-hydroxypyrrolidinyl).

In certain embodiments, R⁹ is halogen. A particular example is fluoro.

In certain embodiments, R⁹ is CF₃.

In certain embodiments, R⁹ is CO₂(1-6C alkyl). An example is CO₂Me.

In certain embodiments, A is a 5-8 membered heterocyclic ring which is unsubstituted or substituted with one or more R⁹ groups independently selected from (1-6C)alkyl, NR^{a}R^{b}, OR^{a}, (1-6C alkyl)OR^{a}, C(O)NR^{a}R^{c}, -(1-6C alkyl)NR^{a}R^{c}, halogen, CO₂(1-6C alkyl), and CF₃.

In certain embodiments, A is a 5-8 membered heterocyclic ring which is unsubstituted or substituted with one or more R⁹ groups independently selected from methyl, NH₂, NMe₂, —NHCH(CH₃)CH₂F, NHCH₂CH₂OCH₃, —NHCH₂CH₂OH, N(CH₃)CH₂CH₂OH, 1-azetidin-3-ol, OH, CH₂OH, C(O)NHMe, CH₂NH₂, CH₂CH₂NMe₂, F, CO₂Me and CF₃. In certain embodiments, A is a 5-6 membered heterocyclic ring having 1-2 ring nitrogen atoms optionally substituted with said R⁹ groups. In particular embodiments, A is a piperidinyl, piperazinyl or pyrrolidinyl ring optionally substituted with one or more of said R⁹ groups.

In certain embodiments, A is a 5-8 membered heterocyclic ring which is unsubstituted or substituted with one or more R⁹ groups independently selected from methyl, NH₂, F and CH₂OH. In certain embodiments, A is a 5-6 membered heterocyclic ring having 1-2 ring nitrogen atoms optionally substituted with said R⁹ groups. In particular embodiments, A is a piperidinyl, piperazinyl or pyrrolidinyl ring optionally substituted with one or more of said R⁹ groups.

In particular embodiments, A is a 5-8 membered heterocyclic ring which is unsubstituted or substituted with one or more groups independently selected from NH₂, NMe₂, Me, OH, CH₂OH, C(O)NHMe, CH₂NH₂, and CH₂CH₂NH₂.

In further embodiments, A is a 5-8 membered heterocyclic ring which is substituted with one or more groups independently selected from methyl, NH₂, NHCH(CH₃)CH₂F, OH, CH₂OH, and F. In certain embodiments, A is a 5-6 membered heterocyclic ring having 1-2 ring nitrogen atoms optionally substituted with said R⁹ groups.

In further embodiments, A is a 5-8 membered heterocyclic ring which is substituted with one or more groups independently selected from F, NH₂ methyl and CH₂OH. In particular embodiments, A is substituted with one or more groups independently selected from F, NH₂ and CH₂OH. In certain embodiments, A is a 5-6 membered heterocyclic ring having 1-2 ring nitrogen atoms optionally substituted with said R⁹ groups.

In other embodiments, A is a 5-8 membered heterocyclic ring which is unsubstituted or substituted with one or more groups independently selected from NH-cyclopropyl, NH(t-butyl), NHMe, NHCH₂C(CH₃)₂OH, NHCH₂(pyrid-2-yl), NHCH₂(pyrid-4-yl), oxo, CH(NH₂)CH₂OH, C(O)C(CH₃)₂NH₂, C(O)CH(CH₃)NH₂, C(O)CH₂NH₂, C(O)CH₂NMe₂, C(O)C(cyclopropylidine)NH₂, CH₂CH₂NHMe, CH₂NMe₂, CH₂NH-cyclopropyl, CH₂NHMe, CH₂-(4-methylpiperazinyl), CH₂(3-hydroxypyrrolidinyl), NHC(O)Me, NHC(O)NH₂, NHC(O)CH₂NH₂, NHC(O)CH(CH₃)NH₂, NHC(O)C(CH₃)₂NH₂, CH₂(pyrid-2-yl), and CH₂(pyrid-4-yl). In particular embodiments, A is a piperidinyl, piperazinyl or pyrrolidinyl ring optionally substituted with one or more of said R⁹ groups.

Particular embodiments of A when represented by a 5-6 membered heterocyclic ring optionally substituted with one or more R⁹ groups include the structures:

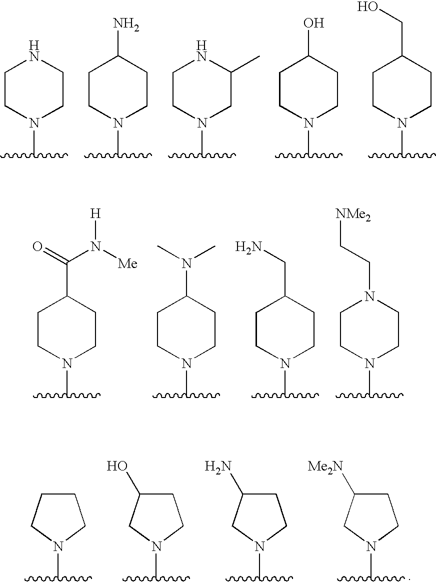

Further particular examples of A when represented by a 5-6 membered heterocyclic ring optionally substituted with one or more R⁹ groups include the structures:

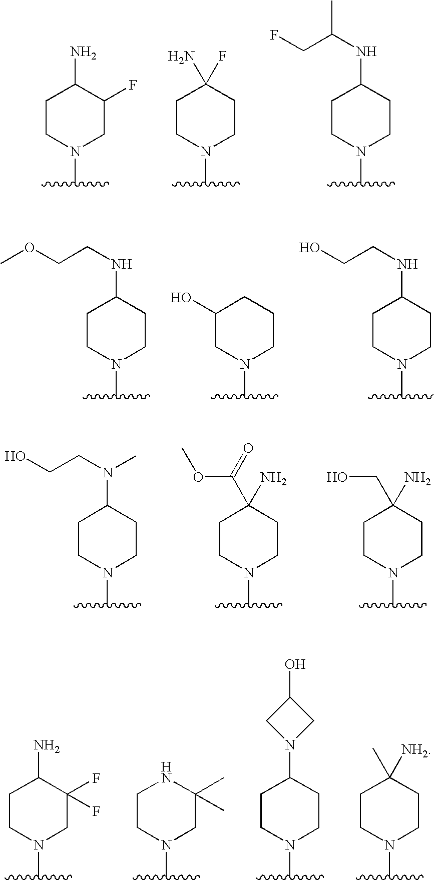

Further exemplary embodiments of A when represented by an optionally substituted 5-6 membered heterocyclic ring include the structures:

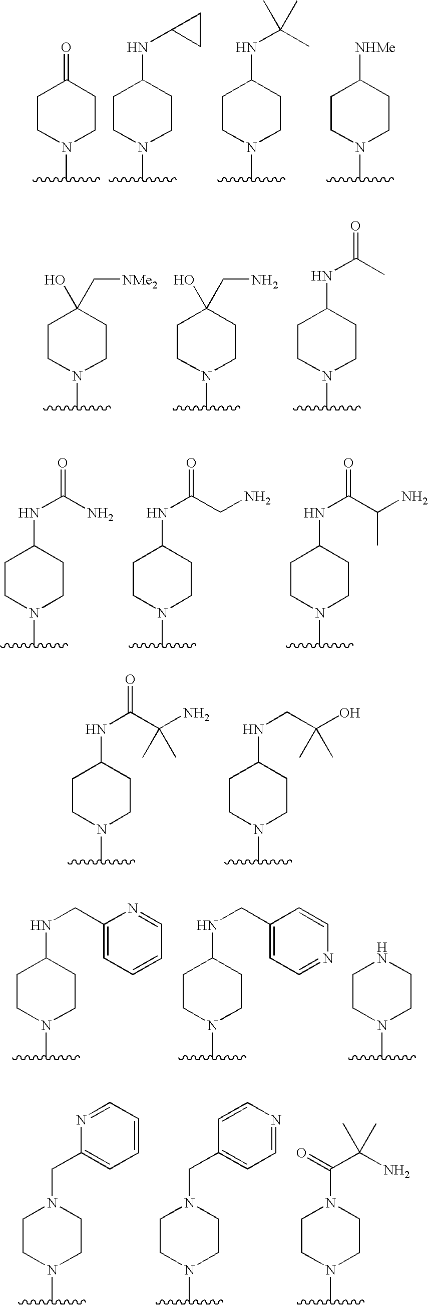

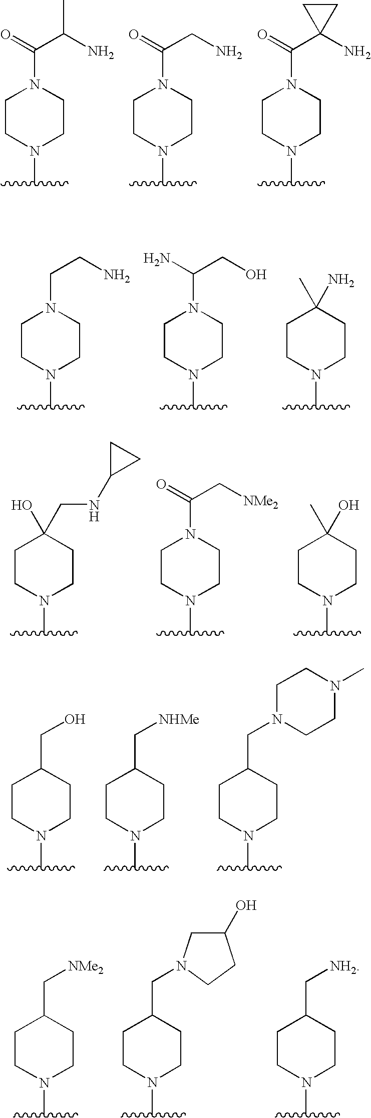

In certain embodiments of Formula I, the A group is selected from groups having the following structures:

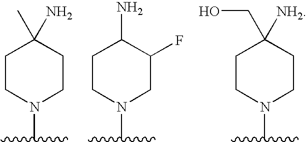

In particular embodiments of Formula I, the A group is selected from groups having the following structures:

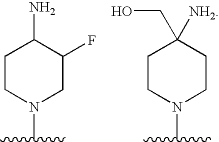

In further particular embodiments of Formula I, the A group is a group having the following structure:

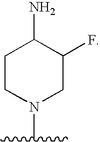

In certain embodiments, the amino and fluoro substituents on the piperidine ring are in the cis-configuration.

In further particular embodiments of Formula I, the A group is a group having the following structure:

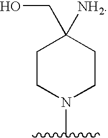

In certain embodiments, B is CN.

In certain embodiments, B is H.

In certain embodiments, B is OR^{h}.

In certain embodiments, B is represented by OR^{h} wherein R^{h} is H.

In certain embodiments, B is represented by OR^{h} wherein R^{h} is CF₃.

In certain embodiments, B is represented by OR^{h} wherein R^{h} is (1-6C)alkyl. Particular values for OR^{h} when R^{h} is represented by (1-6C)alkyl include OMe, OEt and O-isobutyl.

In certain embodiments, B is represented by OR^{h} wherein R^{h} is -(1-6C alkyl)-(3-6C cycloalkyl). Particular values for OR^{h} when R^{h} is represented by -(1-6Calkyl)-(3-6C cycloalkyl) include —O-(1-6Calkyl)-cyclopropyl, for example —OCH₂-cyclopropyl.

In certain embodiments, B is represented by OR^{h} wherein R^{h} is -(1-6C alkyl)-O-(1-6C alkyl). Particular values for OR^{h} when R^{h} is represented by -(1-6C alkyl)-O-(1-6C alkyl) include —OCH₂CH₂OMe and —OCH₂CH₂CH₂OMe.

[symbol]

In certain embodiments, B is represented by OR^{h} wherein R^{h} is -(1-6C alkyl)OH. Particular values for OR^{h} when R^{h} is represented by -(1-6C alkyl)OH include —OCH₂CH₂OH.

In certain embodiments, B is represented by OR^{h} wherein R^{h} is -(1-6C alkyl)-S-(1-6C alkyl). A particular value for OR^{h} when R^{h} is represented by -(1-6C alkyl)-S-(1-6C alkyl) includes —OCH₂CH₂CH₂SMe.

In certain embodiments, B is represented by OR^{h} wherein R^{h} is -(1-6C alkyl)NR′R″. Particular values for OR^{h} when R^{h} is represented by -(1-6C alkyl)NR′R″ include groups wherein R′ and R″ are independently H or Me, for example, —OCH₂CH₂CH₂NH₂, —OCH₂CH₂NMe₂, and —OCH₂CH₂NMe₂. Further examples of OR^{h} include —OCH₂CH₂NH₂, —OCH₂CH₂CH₂NMe₂ and —OCH₂CH₂NHMe.

In certain embodiments, B is represented by OR^{h} wherein R^{h} is hetCyc⁴. Particular values for OR^{h} when R^{h} is represented by hetCyc⁴ include groups wherein hetCyc⁴ is a 5-6 membered heterocyclic ring having 1-2 atoms independently selected from N and O, for example, tetrahydrofuranyl and tetrahydropyranyl rings. Particular examples of OR^{h} include the structures:

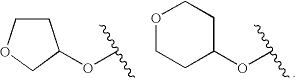

In certain embodiments, B is represented by OR^{h} wherein R^{h} is (1-6C alkyl)hetCyc⁴. Particular values for OR^{h} when R^{h} is represented by (1-6C alkyl)hetCyc⁴ include groups wherein hetCyc⁴ is a 5-6 membered heterocyclic ring having 1-2 atoms independently selected from N and O. A particular example of OR^{h} includes the structure:

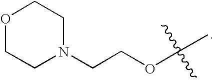

In certain embodiments, B is represented by OR^{h} wherein R^{h} is (1-6C alkyl)aryl. Particular values for OR^{h} when R^{h} is represented by (1-6C alkyl)aryl include groups wherein the aryl is a phenyl group, such as OCH₂Ph.

In certain embodiments, B is represented by OR^{h} wherein R^{h} is (1-6C alkyl)herAr⁵. Particular values for OR^{h} when R^{h} is represented by (1-6C alkyl)-hetAr⁵ include groups wherein herAr⁵ is a 5-6 membered heteroaryl ring having 1-3 nitrogen atoms. Examples include pyridyl, triazolyl and pyrazolyl rings. In certain embodiments, hetAr⁵ is substituted with a group selected from (1-6C) alkyl. Particular examples of OR^{h} include the structures:

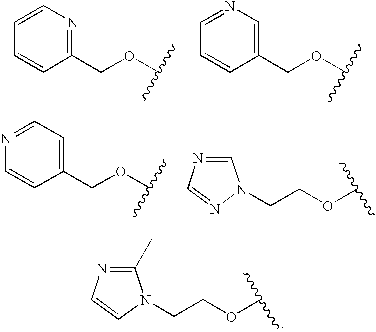

In certain embodiments, B is C(O)NRⁱR^{j}. In certain embodiments, Rⁱ is H. In certain embodiments, Rⁱ is (1-6C alkyl). In certain embodiments, R^{j} is (1-6C alkyl), for example methyl. In other embodiments, R^{j} is (1-6C alkyl)O(1-6 alkyl), for example (1-6C alkyl)OMe. In other embodiments, R^{j} is (1-6C alkyl)OH for example (1-6C alkyl)OH. Particular values for B include —C(O)NHMe, —C(O)NHCH₂CH₂OMe, and —C(O)NHCH₂CH₂OH. In certain embodiments, B is C(O)N(1-6C alkyl)₂. A particular example includes —C(O)NMe₂.

In certain embodiments, B is C(O)-hetCyc³. Examples of hetCyc³ include 5-6 membered heterocyclic rings having 1-2 atoms independently selected from N and O and optionally substituted with OH or O-(1-6C alkyl), for example, optionally substituted piperidinyl, morpholinyl and pyrrolidinyl rings. Particular values for B include the structures:

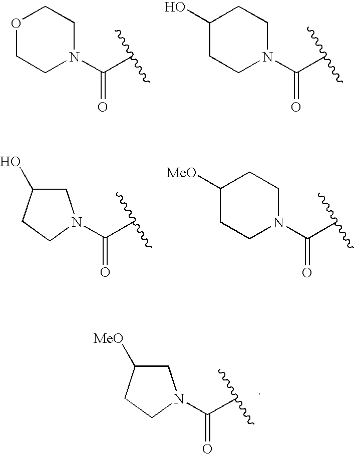

In certain embodiments, B is C(O)(1-6C alkyl)hetCyc³. In other embodiments, B is C(O)NH(1-6C alkyl)hetCyc³. Examples of hetCyc³ include 5-6 membered heterocyclic rings having 1-2 atoms independently selected from N and O. An example of hetCyc³ includes a morpholinyl ring. In certain embodiments, hetCyc³ is substituted with OH or OMe. A particular value for B includes the structure:

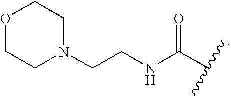

In certain embodiments, B is hetAr². Examples of hetAr² include 5-6 membered heteroaryl rings having 1-2 nitrogen atoms. Examples include pyridyl and pyrimidyl rings. In certain embodiments, hetAr² is substituted with —O(1-6C alkyl), such as methoxy. In certain embodiments, hetAr² is substituted with (1-6C) alkyl. Particular values for B include 3-pyridyl, 4-pyridyl, and 4-methoxypyridy-3-yl. Additional example of hetAr² include 2-pyridyl and 2-pyrimidyl.

In certain embodiments, B is SR^{k}. In certain embodiments, R^{k} is a 3-6 membered carbocyclic ring. In other embodiments, R^{k} is -(1-6C alkyl)O(1-6C alkyl), e.g., (1-6C alkyl)OCH₃. Particular values for B include S-cyclohexyl and S(CH₂CH₂)OCH₃.

In certain embodiments, B is Ar¹. In certain embodiments, Ar¹ is phenyl which is unsubstituted or substituted with OH, O-(1-6C alkyl), C(O)₂(1-6C alkyl), or (1-6C alkyl)NR′R″. Particular values for B include phenyl, phenoxy, 3-methoxyphenyl, 4-(methylamino)phenyl, or 4-(methoxycarbonyl)phenyl.

In certain embodiments, B is -(1-6 alkyl)NR′R″. Particular values include CH₂NHMe and CH₂NMe₂.

In certain embodiments, B is —SO₂N(1-6 alkyl)₂, for example SO₂NMe₂.

In certain embodiments, B is C(O)O(1-6C alkyl), for example C(O)OMe.

In certain embodiments of Formula I, B is selected from H, CN, OR^{h}, hetAr², C(O)NRⁱR^{j}, and CO₂(1-6 C alkyl).

In certain embodiments of Formula I, B is selected from H, CN, —O(1-6C alkyl)-O-(1-6C alkyl), —O(1-6C alkyl)OH, —O(1-6Calkyl)-(3-6C cycloalkyl), —O(1-6C alkyl)NR′R″, a pyridyl ring, or a pyrimidyl ring, C(O)N(di-1-6C alkyl), and CO₂(1-6 C alkyl).

In certain embodiments of Formula I, B is selected from H, CN, —OCH₂CH₂OMe, —OCH₂CH₂OH, —OCH₂(cyclopropyl), 2-pyridyl, 3-pyridyl, 2-pyrimidyl, —OCH₂CH₂NH₂, C(O)NMe₂, and C(O)₂Me.

In certain embodiments of Formula I, B is selected from OR^{h} and hetAr².

In certain embodiments, B is selected from —O(1-6C alkyl)-O-(1-6C alkyl), —O(1-6Calkyl)-(3-6C cycloalkyl), —O(1-6C alkyl)OH, a pyridyl ring, and a pyrimidyl ring.

In certain embodiments, B is —OCH₂CH₂OMe, —OCH₂CH₂OH, —OCH₂(cyclopropyl), 2-pyridyl, 3-pyridyl, or 2-pyrimidyl.

In certain embodiments of Formula I, B is OR^{h}.

In certain embodiments, R^{h} is (1-6C alkyl)-O-(1-6C alkyl), (1-6Calkyl)-(3-6C cycloalkyl), or (1-6C alkyl)OH.

In certain embodiments, B is —OCH₂CH₂OMe, —OCH₂CH₂OH, or —OCH₂(cyclopropyl).

In certain embodiments, B is —OCH₂CH₂OMe.

In certain embodiments, B is hetAr².

In certain embodiments, B is a pyridyl ring or a pyrimidyl ring.

In certain embodiments, B is 2-pyridyl, 3-pyridyl, or 2-pyrimidyl.

Certain compounds according to the present invention have been found to be class 3 receptor tyrosine kinase inhibitors and are useful in the treatment of cancers, such as hematological cancers (e.g., leukemias such as AML), breast cancer, colon cancer, gliomas, fibrosis (including liver fibrosis and lung fibrosis, and scleroderma.

It will be appreciated that certain compounds according to the invention may contain one or more centers of asymmetry and may therefore be prepared and isolated in a mixture of isomers such as a racemic mixture, or in an enantiomerically pure form.

It will further be appreciated that the compounds of Formula I or their salts may be isolated in the form of solvates, and accordingly that any such solvate is included within the scope of the present invention.

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula I.

The term “halogen” as used herein includes F, Cl, Br, and I.

The term “C₁-C₆ alkyl” as used herein refers to saturated linear or branched-chain monovalent hydrocarbon radicals of one to six carbon atoms, respectively. Examples include, but are not limited to, methyl, ethyl, 1-propyl, 2-propyl, 1-butyl, 2-methyl-1-propyl, 2-butyl, 2-methyl-2-propyl, 2,2-dimethylpropyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl.

The term “(1-6C)fluoro alkyl” as used herein refers to a C₁-C₆ alkyl group wherein one or more of the hydrogen atoms is replaced by a fluorine atom.

The term “-(1-6C alkyl)-(3-6C cycloalkyl)” refers to a saturated linear or branched-chain monovalent hydrocarbon radical of one to six carbon atoms, wherein one of the hydrogen atoms is replaced with a 3-6 membered cycloalkyl group.

According to another aspect, the present invention provides a process for the preparation a compound of Formula I or a salt thereof as defined herein which comprises:

(a) coupling a corresponding compound having the formula II

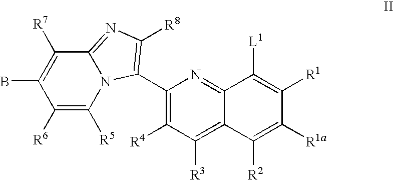

wherein L¹ represents a leaving atom or group, with a compound having the formula HNR¹⁰R¹¹ wherein NR¹⁰R¹¹ forms a 5-8 membered heterocyclic ring optionally substituted with one or more R⁹ groups, using a palladium catalyst and a ligand in the presence of a base; or

(b) for a compound of Formula I where B is OR^{h}, reacting a corresponding compound having the Formula III

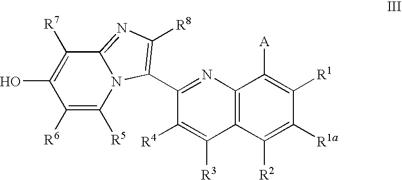

with a compound of the formula R^{h}-L² wherein L² represents a leaving atom or group in the presence of a base; or

(c) for a compound of Formula I where B is OR^{h}, reacting a corresponding compound having the Formula III with a compound having the formula R^{h}—OH in the presence of a coupling reagent; or

(d) for a compound having the Formula I wherein A is:

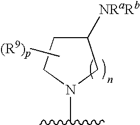

wherein n is 1-3, p is 0-4, R^{b} is other than hydrogen, and R^{a} is as defined for Formula I, reacting a corresponding compound having the formula IV

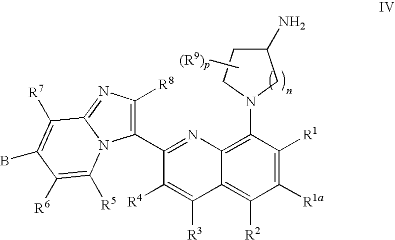

with a compound having the formula R^{a}C(═O)R^{b} where R^{b} is other than hydrogen, followed by treatment with a reducing agent; or

(e) for a compound of Formula I wherein A is:

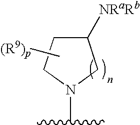

wherein n is 1-3 and p is 0-4, reacting a corresponding compound having the formula V

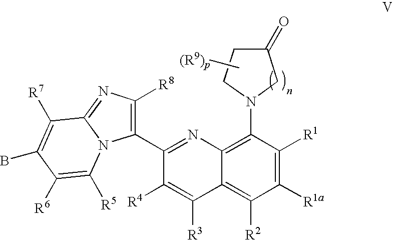

with a compound having the formula HNR^{a}R^{b} followed by treatment with a reducing agent; or

(f) for a compound of Formula I wherein the B group has the formula —O(1-6C alkyl)NH₂, reacting a corresponding compound having the formula VI

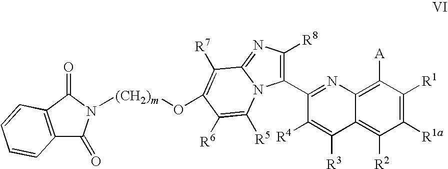

wherein m is an integer from 1-6, with a hydrazine reagent; or

(g) for a compound of Formula I wherein the B group has the formula —O(CH₂CH₂)OH, demethylating a corresponding compound having the formula

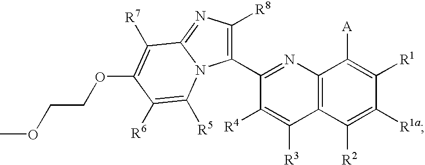

and

removing any protecting group or groups and, if desired, forming a salt.

Referring to method (a), the leaving atom L¹ may be, for example a halogen atom such as Br or I. Alternatively, L¹ can be a leaving group, such as a hydrocarbylsulfonyloxy group, for example, a triflate group, or an arylsulfonyloxy group or an alkylsulfonyloxy group, such as a mesylate or a tosylate group. Suitable palladium catalysts include Pd₂(dba)₃ and Pd(OAc)₂. Suitable ligands include rac-BINAP or DIPHOS. The base may be, for example, an alkali metal carbonate or alkoxide, such as for example cesium carbonate or sodium tert-butoxide. Convenient solvents include aprotic solvents such as ethers (for example tetrahydrofuran or p-dioxane) or toluene. The coupling of a compound of formula (II) with HNR¹⁰R¹¹ can be conveniently performed at a temperature between 0° C. and reflux, and more particularly at reflux.

Referring to method (b), the leaving atom L¹ may be, for example a halogen atom such as Br, Cl or I. Alternatively, L¹ can be a leaving group, for example an arylsulfonyloxy group or an alkylsulfonyloxy group, such as a mesylate or a tosylate group. The base may be, for example, an alkali metal hydride or carbonate, such as sodium hydride, potassium hydride, sodium carbonate, potassium carbonate or cesium carbonate. Convenient solvents include aprotic solvents such as ethers (for example tetrahydrofuran or p-dioxane), DMF, or acetone. The reaction can be conveniently performed at a temperature ranging from −78 to 100° C.

Referring to method (c), the coupling reagent may be any suitable reagent(s) known to those skilled in the art, for example, DEAD and PPh₃. Convenient solvents include aprotic solvents such as ethers (for example tetrahydrofuran). The reaction can be conveniently performed at a temperature ranging from −78 to 100° C.

Referring to methods (d) and (e) suitable reducing agents include metal hydrides such as sodium borohydride.

Referring to method (f), the hydrazine reagent can be hydrazine or a derivative thereof such as methylhydrazine.

Referring to method (g), the demethylation step can take place in the presence of a Lewis acid, such as BBr₃ or BCl₃. The reaction is conveniently performed at reduced temperatures, for example at a temperature ranging from −78 to 0° C. Suitable solvents include aprotic solvents such as dichloromethane.

A compound of Formula II

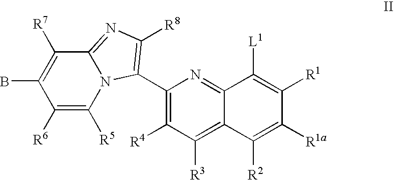

can be prepared by reacting corresponding 2,8-dibromoquinoline having the formula

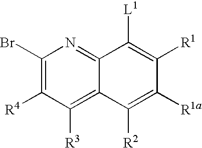

with a corresponding compound having the formula

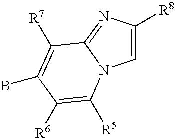

using a palladium catalyst (such as Pd(PPh₃)₄, Pd₂(dba)₃ or Pd(OAc)₂) and a palladium ligand (for example rac-BINAP or DIPHOS) in the presence of a suitable base, for example an alkali metal carbonate or alkoxide base (e.g., cesium carbonate, potassium carbonate, or sodium tert-butoxide) in a suitable solvent (such as toluene or dioxane) at a temperature ranging from about ambient temperature to reflux.

Alternatively, a compound of formula (II) can be prepared by reacting a corresponding compound having the formula (VII)

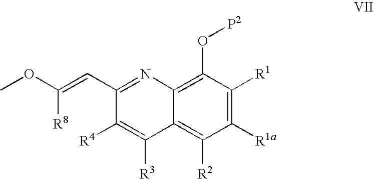

wherein P² represents an alcohol protecting group, such as t-butyldimethylsilyl, with a corresponding compound having the formula

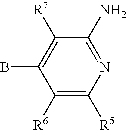

in the presence of N-bromosuccimide or N-chlorosuccinimide in a suitable solvent (such as THF).

The compounds of the formulas (II), (III) and (VII) as shown in the above methods (a), (b) and (c) are believed to be novel and are provided as further aspects of the invention.

The ability of test compounds to act as PDGFR inhibitors may be demonstrated by the assay described in Example A.

The ability of test compounds to act as FLT3 inhibitors may be demonstrated by the assay described in Example B.

Compounds of Formula I are useful for treating diseases and disorders mediated by class 3 and/or class 5 receptor tyrosine kinases. In particular embodiments, compounds of formula I are inhibitors of one or more of the class 3 receptor tyrosine kinases, for example PDGFR and FLT3. For example, compounds of this invention are useful in the treatment fibrosis (including lung, liver and kidney fibroses), scleroderma, and cancers, including hematological malignancies.

As used herein, the term treatment includes prophylaxis as well as treatment of an existing condition.

Examples of hematological malignancies include, for instance, leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

Particular examples of PDGFR-driven or dependent cancers which may be treated with compounds of this invention include dermatofibrosacroma protuberans (DFSB), chronic myelomonocytic leukemia (CMML), hypereosinophilic syndrome (HES), glioblastoma multiforme (GBM) and gastrointestinal stromal tumors (GIST).

FLT3 inhibitors may also be useful for treating immune related disorders such as bone marrow transplant rejection, solid organ rejection after transplant, ankylosing spondylitis, arthritis, aplastic anemia, Behcet's disease, Graves' disease, hemolytic anemia, hyper IgE syndrome, idiopathic thrombocytopenia purpura (ITP), multiple sclerosis (MS), rheumatoid arthritis, Wegener's granulomatosis, type 1 diabetes mellitus, Myasthenia gravis, and psoriasis.

Particular compounds of this invention are inhibitors of Pim-1 and therefore are useful in treating diseases and disorders mediated by Pim-1, such as cancers such as hematological cancers.

Accordingly, another aspect of this invention provides a method of treating diseases or medical conditions in a mammal mediated by a class 3 and/or class 5 receptor tyrosine kinase, comprising administering to said mammal one or more compounds of Formula I or a pharmaceutically acceptable salt or prodrug thereof in an amount effective to treat or prevent said disorder.

Another aspect of this invention provides a method of treating diseases or medical conditions in a mammal mediated by Pim-1, comprising administering to said mammal one or more compounds of Formula I or a pharmaceutically acceptable salt or prodrug thereof in an amount effective to treat or prevent said disorder.

The phrase “effective amount” means an amount of compound that, when administered to a mammal in need of such treatment, is sufficient to (i) treat or prevent a particular disease, condition, or disorder mediated by a class 3 receptor tyrosine kinase, (ii) attenuate, ameliorate, or eliminate one or more symptoms of the particular disease, condition, or disorder, or (iii) prevent or delay the onset of one or more symptoms of the particular disease, condition, or disorder described herein.

The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the present invention can be used in combination with one or more additional drugs, for example an anti-inflammatory compound, anti-fibrotic compound or a chemotherapeutic that works by the same or by a different mechanism of action.

Compounds of the invention may be administered by any convenient route, e.g. into the gastrointestinal tract (e.g. rectally or orally), the nose, lungs, musculature or vasculature, or transdermally or dermally. The compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

According to another aspect, the present invention provides a pharmaceutical composition, which comprises a compound of Formula I or a pharmaceutically acceptable salt thereof, as defined hereinabove. In one embodiment, the pharmaceutical composition includes the compound of Formula I together with a pharmaceutically acceptable diluent or carrier.

According to another aspect, the present invention provides a compound of Formula I or a pharmaceutically acceptable salt thereof, for use in therapy, such as the treatment of a class 3 and/or class 5 receptor tyrosine kinase-mediated condition.

According to a further aspect, the present invention provides the use of a compound of Formula I or a pharmaceutically acceptable salt thereof, in the manufacture of a medicament to treat a class 3 and/or class 5 receptor tyrosine kinase-mediated condition, as defined hereinabove.

In certain embodiments, the invention provides the used of a compound of Formula I or a pharmaceutically acceptable salt thereof, for use in the treatment of cancer.

In certain embodiments, the invention provides the used of a compound of Formula I or a pharmaceutically acceptable salt thereof, for use in the treatment of fibrosis.

In certain embodiments, the invention provides the used of a compound of Formula I or a pharmaceutically acceptable salt thereof, for use in the treatment of scleroderma.

According to another aspect, the present invention provides a compound of Formula I or a pharmaceutically acceptable salt thereof, for use in therapy, such as the treatment of a Pim-1-mediated condition.

According to a further aspect, the present invention provides the use of a compound of Formula I or a pharmaceutically acceptable salt thereof, in the manufacture of a medicament to treat a Pim-1-mediated condition, as defined hereinabove.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), dichloromethane (DCM, methylene chloride), toluene, and dioxane were purchased from Aldrich in Sure seal bottles and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

¹HNMR spectra were obtained as CDCl₃, CD₃OD, D₂O or d₆-DMSO solutions (reported in ppm), using tetramethylsilane (0.00 ppm) or residual solvent (CDCl₃: 7.25 ppm; CD₃OD: 3.31 ppm; D₂O: 4.79 ppm; d₆-DMSO: 2.50 ppm) as the reference standard. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### Example A

### Cellular PDGFR Assay

The ability of compounds of this invention to inhibit PDGF-induced PDGFR phosphorylation was assessed by using mouse NIH3T3 cells.

25,000 cells in DMEM supplemented with 10% fetal bovine serum were added to each well of a black 96-well cell culture plate. Plates were incubated in a 37° C./5% CO₂ incubator for 6-8 hours. Plates were then washed and incubated with serum-free DMEM, and the cells were returned to the 37° C./5% CO₂ incubator for 16-20 hours.

Compound test solutions were added at a final concentration of 0.5% DMSO, and the cells were incubated in a 37° C./5% CO₂ incubator for 1 hour. PDGF-BB ligand was then added (75 ng/mL) and incubated for 15 minutes. Cells were washed with PBS and fixed in 3.7% formaldehyde in PBS for 10 minutes. This was followed by washing in PBS/0.2% Triton X-100 and permeabilizing in 100% MeOH for 10 minutes. Cells were blocked in Odyssey blocking buffer (LI-COR Biosciences) for 1 hour. Antibodies to phosphorylated PDGFRβ and total PDGFRβ were added to the cells and incubated for 3 hours. After washing with PBS/0.2% TritonX-100, the cells were incubated with fluorescently-labeled secondary antibodies (goat anti-rabbit IgG-IRDye800 and goat anti-mouse IgG-Alexa Fluor 680) for an additional hour. Cells were then washed with PBS and analyzed for fluorescence at both wavelengths using the Odyssey Infrared Imaging System (LI-COR Biosciences). Phosphorylated PDGFR signal was normalized to total PDGFR signal. Compounds of the invention were found to have an IC₅₀ less than 10 μM when tested in this assay.

### Example B

### Cellular FLT3 Assay

The inhibition of FLT3 ligand (FL)-induced phosphorylated FLT3 in human RS4; 11 cells was measured as follows. Cells were plated in 96-well V-bottom plates in RPMI/10% FCS at a concentration of 1 million cells/well. Diluted compounds were added at a final concentration of 0.5% DMSO for one hour. FL was added at a final concentration of 50 ng/ml. After a 15 minute incubation, the cells were pelleted by centrifugation and resuspended in lysis buffer. Phospho-FLT3 was detected by standard ELISA procedure (R&D Systems; DYC368). Briefly, after 20 minutes on ice, the lysate was added to 96-well plates coated with capture antibody to total FLT3. Phospho-FLT3 was detected by the addition of antibody to phospho-tyrosine conjugated to HRP. After addition of substrate and stop solution, the signal was read at A450. Compounds of the invention were found to have an IC₅₀ less than 10 μM when tested in this assay.

### Example 1

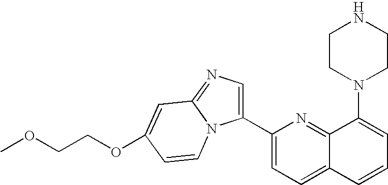

### 2-(7-(2-Methoxyethoxy)imidazo[1,2-a]pyridin-3-yl)-8-(piperazin-1-yl)quinoline

Step 1A: Preparation of 2-chloro-4-(2-methoxyethoxy)pyridine: A mixture of 2-chloro-4-nitropyridine (43.6 g, 275.0 mmol) and 2-methoxyethanol (325.6 ml, 425 mmol) was cooled to 0° C. Potassium 2-methylpropan-2-olate (35.73 g, 302.5 mmol) was added and the resulting mixture was stirred while warming to ambient temp over 2 hours. The reaction mixture was concentrated under reduced pressure followed by dilution with 500 ml of water. The resulting mixture was extracted twice with 250 ml of dichloromethane. The combined organic layers were dried over MgSO₄ and concentrated under reduced pressure to produce the desired compound as a golden oil. (50.2 g, 97% yield) MS APCI (+) m/z 188 and 189.9 (M+1 of each isotope) detected.

Step 1B: Preparation of 4-(2-methoxyethoxy)pyridin-2-amine: A steady stream of nitrogen was passed through a mixture of 2-chloro-4-(2-methoxyethoxy)pyridine (50.17 g, 267.4 mmol), Pd₂ dba₃ (4.897 g, 5.348 mmol), XPHOS (5.099 g, 10.70 mmol) and tetrahydrofuran (445.7 ml) for 10 minutes. To the resulting degassed mixture was added lithium bis(trimethylsilyl)amide (561.5 ml, 561.5 mmol). After addition, the resulting mixture was heated to 60° C. for 18 hours. The reaction was cooled to ambient temperature and diluted with 1 N hydrochloric acid (200 mL). The resulting solution was washed twice with 500 ml of methyl-tert-butyl ether. The pH of the aqueous layer was taken to 11 with 6 N NaOH and was extracted with dichloromethane (3×500 ml). The combined organic layers were dried over MgSO₄ and concentrated under reduced pressure to yield title compound. (35 g, 78% yield) MS APCI (+) m/z 169 (M+1) detected.

Step 1C: Preparation of 7-(2-methoxyethoxy)imidazo[1,2-a]pyridine: A mixture of 4-(2-methoxyethoxy)pyridin-2-amine (20.0 g, 119 mmol), 2-chloroacetaldehyde (32.2 ml, 250 mmol) and tetrahydrofuran (100 mL) were heated in a sealed tube to 75° C. over 3 days. The reaction mixture was concentrated under reduced pressure and dissolved in ethyl acetate. The resulting solution was washed twice sodium bicarbonate. The combined organic layers were dried over MgSO₄ and concentrated under reduced pressure to yield title compound. (23.5 g, quantitative yield) MS APCI (+) m/z 193 (M+1) detected.

Step 2A: Preparation of N-(2-bromophenyl)cinnamamide: To a mixture of 2-bromobenzenamine (200.0 g, 1163 mmol), pyridine (188.1 ml, 2325 mmol) and dry dichloromethane (1000 ml) at 0° C. was added slowly cinnamoyl chloride (193.7 g, 1163 mmol). The resulting mixture was stirred while warming to ambient temperature overnight. The resulting mixture was washed with sodium bicarbonate (1000 ml), 10% sodium bisulfate (1000 ml), sodium bicarbonate (1000 ml) and brine (1000 ml). The organic layer was dried over MgSO₄ and concentrated under reduced pressure to yield title compound as a solid (172.3 gm, 98% yield) MS ESI (+) m/z 224 and 226 (M+1 of each isotope) detected.

Step 2B: Preparation of 8-bromoquinolin-2-one: A mixture of N-(2-bromophenyl)cinnamamide (172.3 g, 570.3 mmol), aluminum chloride (456 g, 342 mmol) and chlorobenzene (1000 ml) were allowed to stir at 100° C. for 7 hours followed by cooling to ambient temperature overnight. The resulting mixture was poured onto 2 kg of ice and was allowed to warm to ambient temperature over 1 hour. The resulting mixture was extracted with dichloromethane. The combined organic layers were dried over MgSO₄ and concentrated under reduced pressure. The resulting solids were triturated with 1000 ml hexanes. The solids were vacuum dried to yield title compound. (83 g, 65% yield) MS ESI (+) m/z 224 and 226 (M+1 of each isotope) detected.

Step 2C: Preparation of 2,8-dibromoquinoline: A mixture of 8-bromoquinolin-2(1H)-one (5 g, 22 mmol) and phosphoryl tribromide (13 g, 45 mmol) was heated to 140° C. for three hours. The resulting mixture was poured onto 100 g of ice and 100 ml water. The mixture was stirred for 1 hour and the resulting solids were filtered to yield the title compound. (5.1 g, 80% yield) MS APCI (+) 286, 288, and 290 (M+1 of each isotope combination) detected.

Step D: Preparation of 8-bromo-2-(7-(2-methoxyethoxy)imidazo[1,2-a]pyridin-3-yl)quinoline: A mixture of 2,8-dibromoquinoline (22.4 g, 78.0 mmol), 7-(2-methoxyethoxy)imidazo[1,2-a]pyridine (15.0 g, 78.0 mmol), Pd(PPh₃)₄ (4.51 g, 3.90 mmol), K₂CO₃ (21.6 g, 156 mmol) and Pd(OAc)₂ (0.876 g, 3.90 mmol), dioxane (312 mL) and water (3 ml) was heated to 100° C. for 18 hours. The resulting mixture was diluted with dichloromethane (500 ml) and filtered. The filtrate was concentrated under reduced pressure and to the resulting oil was added ethyl acetate (100 ml) and methyl tert-butyl ether (100 ml). The resulting mixture was stirred overnight. Filtration to collect the resulting solids yielded the title compound (22.2 g, 72% yield). MS ESI (+) m/z 398 and 400 (M+1 of each isotope) detected.

Step E: Preparation of tert-butyl 4-(2-(7-(2-methoxyethoxy)imidazo[1,2-a]pyridin-3-yl)quinolin-8-yl)piperazine-1-carboxylate: A stream of argon was passed through a mixture of 8-bromo-2-(7-(2-methoxyethoxy)H-imidazo[1,2-a]pyridin-3-yl)quinoline (20 g, 50 mmol), tert-butyl piperazine-1-carboxylate (18.7 g, 100 mmol), Cs₂CO₃ (81.8 g, 251 mmol), Pd₂(dba)₃ (2.3 g, 2.51 mmol), rac-BINAP (3.1 g, 5.0 mmol) in toluene (800 ml) for 15 minutes. The mixture was heated to 100° C. for 18 hours. The mixture was then allowed to cool to ambient temperature and dichloromethane (1000 ml) was added. After stirring 30 minutes, the resulting mixture was filtered and the filtrate was concentrated to yield an oil. The resulting oil was chromatographed on silica gel to yield the title compound. (5.5 g, 21% yield). MS APCI (+) m/z 505 (M+1) detected.

Step F: Preparation of 2-(7-(2-methoxyethoxy)imidazo[1,2-a]pyridin-3-yl)-8-(piperazin-1-yl)quinoline: To a solution of tert-butyl 4-(2-(7-(2-methoxyethoxy)imidazo[1,2-a]pyridin-3-yl)quinolin-8-yl)piperazine-1-carboxylate (5.5 g) in 50 ml dichloromethane was added 50 ml trifluoroacetic acid. The resulting mixture was stirred for 2 hours at ambient temperature. Reaction mixture was concentrated under reduced pressure and then diluted with 100 ml dichloromethane. The resulting solution was washed twice with 100 ml saturated sodium bicarbonate and twice with 100 ml of a brine solution. The organic layer was dried over magnesium sulfate, filtered and concentrated under reduced pressure to produce the title compound. (4.4 g, 100% Yield) MS APCI (+) m/z 404.3 (M+1) detected.

### Example 2

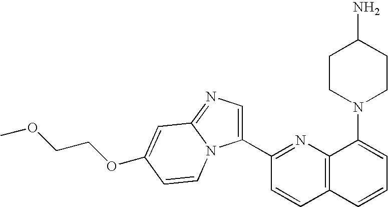

### 1-(2-(7-(2-Methoxyethoxy)imidazo[1,2-a]pyridin-3-yl)quinolin-8-yl)piperidin-4-amine

Prepared according to the procedure for Example 1 using tert-butyl piperidin-4-ylcarbamate in place of tert-butyl piperazine-1-carboxylate. MS APCI (+) m/z 418.3 (M+) detected.

### Example 3

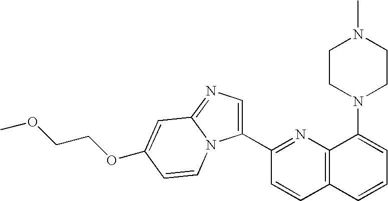

### 2-(7-(2-Methoxyethoxy)imidazo[1,2-a]pyridin-3-yl)-8-(4-methylpiperazin-1-yl)quinoline

Prepared according to the procedure for Example 1 using 1-methylpiperazine in place of tert-butyl piperazine-1-carboxylate. MS APCI (+) m/z 418.3 (M+1) detected.

### Example 4

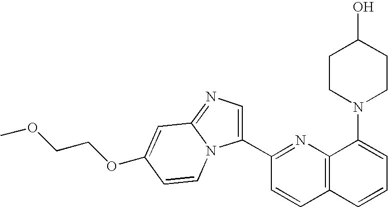

### 1-(2-(7-(2-Methoxyethoxy)imidazo[1,2-a]pyridin-3-yl)quinolin-8-yl)piperidin-4-ol

Prepared according to the method of Example 1. MS ESI (+) m/z 419.3 (M+1) detected.

### Example 5

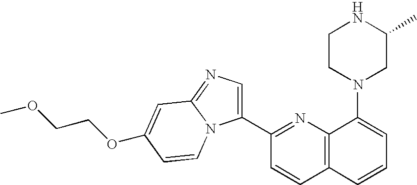

### (R)-2-(7-(2-Methoxyethoxy)imidazo[1,2-a]pyridin-3-yl)-8-(3-methylpiperazin-1-yl)quinoline

Prepared according to the method of Example 1. MS ESI (+) m/z 418.3 (M+1) detected.

### Example 6

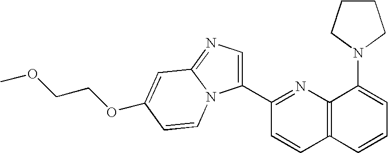

### 2-(7-(2-Methoxyethoxy)imidazo[1,2-a]pyridin-3-yl)-8-(pyrrolidin-1-yl)quinoline

Prepared according to the method of Example 1. MS APCI (+) m/z 489.2 (M+1) detected.

### Example 7

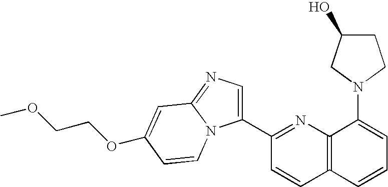

### (S)-1-(2-(7-(2-Methoxyethoxy)imidazo[1,2-a]pyridin-3-yl)quinolin-8-yl)pyrrolidin-3-ol

Prepared according to the method of Example 1. MS ESI (+) m/z 405.3 (M+1) detected.

### Example 8

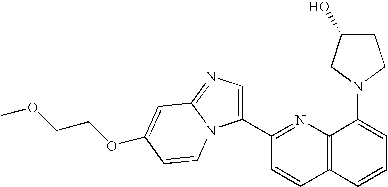

### (R)-1-(2-(7-(2-Methoxyethoxy)imidazo[1,2-a]pyridin-3-yl)quinolin-8-yl)pyrrolidin-3-ol

Prepared according to the method of Example 1. MS ESI (+) m/z 405.3 (M+1) detected.

### Example 9

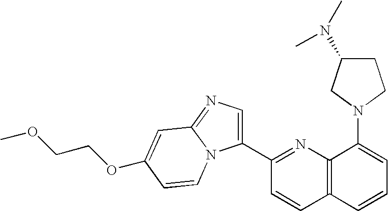

### (R)-1-(2-(7-(2-Methoxyethoxy)imidazo[1,2-a]pyridin-3-yl)quinolin-8-yl)-N,N-dimethylpyrrolidin-3-amine

Prepared according to the method of Example 1. MS ESI (+) m/z 432.2 (M+1) detected.

### Example 10

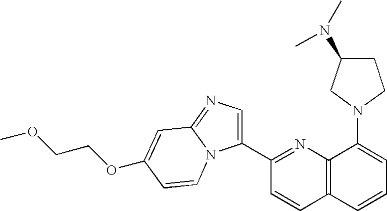

### (S)-1-(2-(7-(2-Methoxyethoxy)imidazo[1,2-a]pyridin-3-yl)quinolin-8-yl)-N,N-dimethylpyrrolidin-3-amine

Prepared according to the method of Example 1. MS ESI (+) m/z 432.2 (M+1) detected.

### Example 11

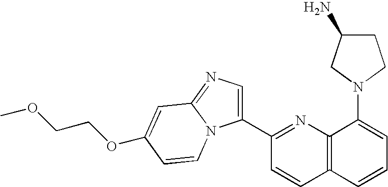

### (S)-1-(2-(7-(2-Methoxyethoxy)imidazo[1,2-a]pyridin-3-yl)quinolin-8-yl)pyrrolidin-3-amine

Prepared according to the method of Example 1. MS ESI (+) m/z 404.3 (M+1) detected.

### Example 12

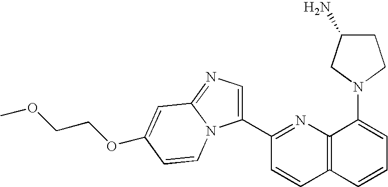

### (R)-1-(2-(7-(2-Methoxyethoxy)imidazo[1,2-a]pyridin-3-yl)quinolin-8-yl)pyrrolidin-3-amine

Prepared according to the method of Example 1. MS ESI (+) m/z 404.3 (M+1) detected.

### Example 13

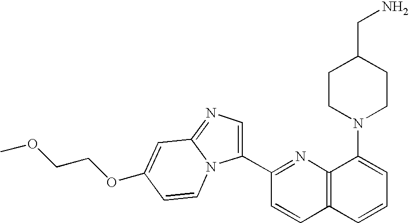

### (1-(2-(7-(2-Methoxyethoxy)imidazo[1,2-a]pyridin-3-yl)quinolin-8-yl)piperidin-4-yl)methanamine

Prepared according to the method of Example 1. MS ESI (+) m/z 432.3 (M+1) detected.

### Example 14

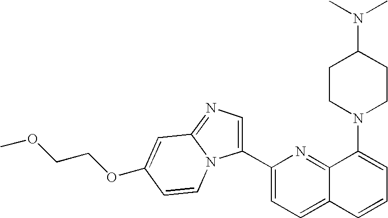

### 1-(2-(7-(2-Methoxyethoxy)imidazo[1,2-a]pyridin-3-yl)quinolin-8-yl)-N,N-dimethylpiperidin-4-amine

Prepared according to the method of Example 1. MS ESI (+) m/z 446.2 (M+1) detected.

### Example 15

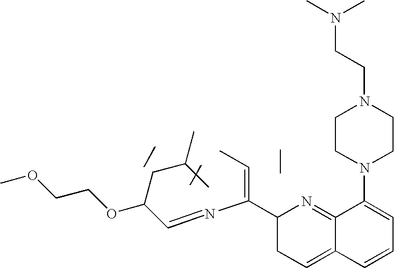

### 2-(4-(2-(7-(2-Methoxyethoxy)imidazo[1,2-a]pyridin-3-yl)quinolin-8-yl)piperazin-1-yl)-N,N-dimethylethanamine

Prepared according to the method of Example 1. MS ESI (+) m/z 475.2 (M+1) detected.

### Example 16

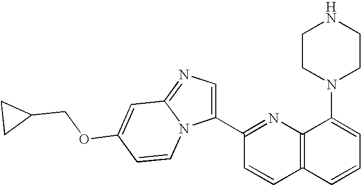

### 2-(7-(Cyclopropylmethoxy)imidazo[1,2-a]pyridin-3-yl)-8-(piperazin-1-yl)quinoline

Prepared according to the procedure for Example 1 using cyclopropylmethanol in place of 2-methoxyethanol. MS ESI (+) m/z 400.2 (M+1) detected.

[symbol]

### Example 17

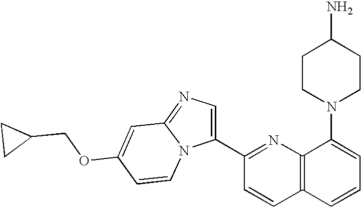

### 1-(2-(7-(Cyclopropylmethoxy)imidazo[1,2-a]pyridin-3-yl)quinolin-8-yl)piperidin-4-amine

Prepared according to the methods of Examples 1 and 23. MS ESI (+) m/z 414.2 (M+1) detected.

### Example 18

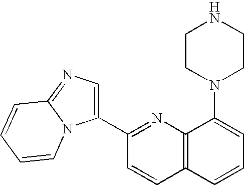

### 2-(Imidazo[1,2-a]pyridin-3-yl)-8-(piperazin-1-yl)quinoline

Prepared according to the procedure for Example 1 using imidazo[1,2-a]pyridine in place of 7-(2-methoxyethoxy)imidazo[1,2-a]pyridine. MS APCI (+) m/z 330.2 (M+1) detected.

### Example 19

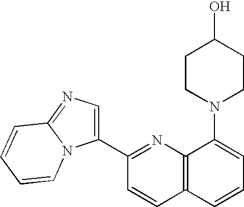

### 1-(2-Imidazo[1,2-a]pyridin-3-yl)quinolin-8-yl)piperidin-4-ol

Prepared according to the procedure for Example 1 using imidazo[1,2-a]pyridine in place of 7-(2-methoxyethoxy)imidazo[1,2-a]pyridine and piperidin-4-ol in place of tert-butyl piperazine-1-carboxylate. MS APCI (+) m/z 345.3 (M+1) detected.

### Example 20

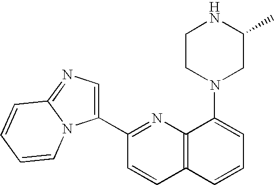

### (R)-2-(Imidazo[1,2-a]pyridin-3-yl)-8-(3-methylpiperazin-1-yl)quinoline

Prepared according to the procedure for Example 1 using imidazo[1,2-a]pyridine in place of 7-(2-methoxyethoxy)imidazo[1,2-a]pyridine and (R)-2-methylpiperazine in place of tert-butyl piperazine-1-carboxylate. MS APCI (+) m/z 344.3 (M+1) detected.

### Example 21

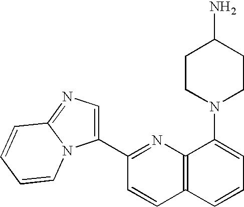

### 1-(2-(Imidazo[1,2-a]pyridin-3-yl)quinolin-8-yl)piperidin-4-amine

Prepared according to the procedure for Example 1 using imidazo[1,2-a]pyridine in place of 7-(2-methoxyethoxy)imidazo[1,2-a]pyridine and tert-butyl piperidin-4-ylcarbamate in place of tert-butyl piperazine-1-carboxylate. MS APCI (+) m/z 344.2 (M+1) detected.

### Example 22

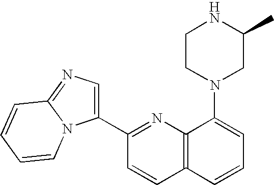

### (S)-2-(Imidazo[1,2-a]pyridin-3-yl)-8-(3-methylpiperazin-1-yl)quinoline

Prepared according to the procedure for Example 1 using imidazo[1,2-a]pyridine in place of 7-(2-methoxyethoxy)imidazo[1,2-a]pyridine and (S)-2-methylpiperazine in place of tert-butyl piperazine-1-carboxylate. MS APCI (+) m/z 344.3 (M+1) detected.

### Example 23

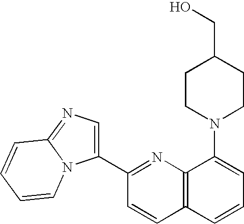

### (1-(2-(Imidazo[1,2-a]pyridin-3-yl)quinolin-8-yl)piperidin-4-yl)methanol

Prepared according to the procedure for Example 1 using imidazo[1,2-a]pyridine in place of 7-(2-methoxyethoxy)imidazo[1,2-a]pyridine and piperidin-4-ylmethanol in place of tert-butyl piperazine-1-carboxylate. MS APCI (+) m/z 359.4 (M+1) detected.

### Example 24

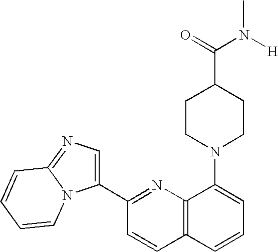

### 1-(2-(Imidazo[1,2-a]pyridin-3-yl)quinolin-8-yl)-N-methylpiperidine-4-carboxamide

Prepared according to the procedure for Example 1 using imidazo[1,2-a]pyridine in place of 7-(2-methoxyethoxy)imidazo[1,2-a]pyridine and N-methylpiperidine-4-carboxamide in place of tert-butyl piperazine-1-carboxylate. MS APCI (+) m/z 386.3 (M+1) detected.

### Example 25

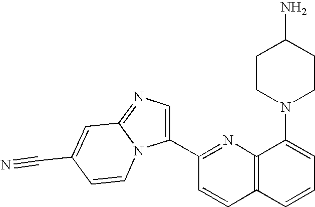

### 3-(8-(4-Aminopiperidin-1-yl)quinolin-2-yl) imidazo[1,2-a]pyridine-7-carbonitrile

Prepared according to the procedure for Example 1 using 2-aminoisonicotinonitrile in place of 4-(2-methoxyethoxy)pyridin-2-amine and tert-butyl piperidin-4-ylcarbamate in place of tert-butyl piperazine-1-carboxylate. MS APCI (+) m/z 369.2 (M+1) detected.

### Example 26

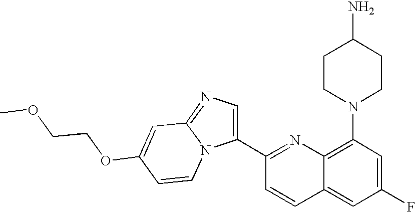

### 1-(6-Fluoro-2-(7-(2-methoxyethoxy)imidazo[1,2-a]pyridin-3-yl)quinolin-8-yl)piperidin-4-amine

Step A: Preparation of 8-bromo-6-fluoro-2-methylquinoline: 2-Bromo-4-fluorobenzenamine (10 g, 52.6 mmol) was weighed into a 100 mL flask and dissolved in 40 mL of 6 N HCl. The reaction mixture was heated to reflux, followed by dropwise addition of (E)-but-2-enal (4.578 ml, 55.3 mmol) mixed with 1.0 mL deionized water over 25 minutes. Following complete addition, the reaction was heated at 100° C. for an additional 35 minutes. The reaction was cooled to ambient temperature, followed by addition of 50 mL of Et₂O. The reaction was stirred for 5 minutes followed by removal of Et₂O by partitioning. The aqueous layer was replaced into the original reaction flask and ZnCl₂ (3.5865 g, 26.3 mmol) was then added in two portions followed by cooling to 0° C. over 30 minutes. The pH of the crude reaction mixture was then adjusted to pH=8.0 using concentrated NH₄OH. The crude mixture was then extracted with Et₂O, followed by ethyl acetate. The combined organics were then dried over Na₂SO₄, then filtered and concentrated in vacuo, affording the desired product as a solid. (10.7 g, 85% yield) MS APCI (+) m/z 240.2 and 242.2 (M+1 of each isotope) detected.

Step B: Preparation of 8-bromo-2-(dibromomethyl)-6-fluoroquinoline: 8-Bromo-6-fluoro-2-methylquinoline (10.7 g, 44.6 mmol) was weighed into a 1000 mL flask, followed by addition of NaOAc (21.9 g, 267 mmol). The solids were suspended in 500 mL of AcOH, and the reaction heated to 70° C. Bromine (6.85 mL, 134 mmol) was the added dropwise over 25 minutes as a solution in 30 mL of AcOH. Following complete addition, the reaction was stirred at 100° C. for 1 hour. The reaction was cooled to ambient temperature, then poured onto 750 cc of ice. The ice was allowed to melt completely and the slurry was separated by partitioning into ethyl acetate. The combined organics were dried over magnesium sulfate, then filtered and concentrated in vacuo to afford a solid. (17.2 g, 97% yield).

Step C: Preparation of Ethyl-8-bromo-6-fluoroquinoline-2-carboxylate and 8-bromo-6-fluoroquinoline-2-carboxylic acid: 8-Bromo-2-(dibromomethyl)-6-fluoroquinoline (17.2 g, 43.2 mmol) was weighed into a 1000 mL and dissolved in 250 mL of EtOH, followed by addition of silver nitrate (23.5 g, 138 mmol) in 100 mL of 1:1 EtOH/H₂O. The reaction was then heated to reflux for 1 hour. The reaction was removed from heat and filtered hot through a medium frit sintered glass funnel, affording 5.84 g of 8-bromo-6-fluoroquinoline-2-carboxylic acid. The mother liquor was then concentrated in vacuo, followed by extractive work-up (200 mL ethyl acetate/water), then washed with ethyl acetate. The combined organic phase was dried over Na₂SO₄, then filtered and concentrated in vacuo to afford 6.4 g of ethyl 8-bromo-6-fluoroquinoline-2-carboxylate as a semi-solid. MS APCI (+) m/z 298 and 300 (M+1 of each isotope) detected.

Step D: Preparation of (8-bromo-6-fluoroquinolin-2-yl)methanol: Ethyl 8-bromo-6-fluoroquinoline-2-carboxylate (3.201 g, 10.7 mmol) was weighed into a 500 mL flask and dissolved in 100 mL of DCM. The reaction was cooled to −78° C., followed by dropwise addition of DIBAL-H (21.48 ml, 32.22 mmol) over 10 minutes. The reaction was allowed to stir and warm to ambient temperature over 2 hours. The reaction was then quenched with 10 mL MeOH, followed by addition of 100 mL of Rochelle's salts, then stirred overnight. The reaction was partitioned with ethyl acetate, and the organic fractions combined and concentrated in vacuo. The crude semi-solid was purified by flash column chromatography (eluting with a 20-50% ethyl acetate/hexane gradient), affording the desired product as a semi-solid. (2.27 g, 42% yield). MS APCI (+) m/z 256.1 and 258 (M+1 of each isotope) detected.

Step E: Preparation of 8-bromo-6-fluoroquinoline-2-carbaldehyde: (8-Bromo-6-fluoroquinolin-2-yl)methanol (2 g, 7.8 mmol), DMSO (8.9 ml, 125.0 mmol), and triethylamine (4.9 ml, 35 mmol) were weighed into a 100 mL flask and dissolved in a 10 mL of DCM, followed by cooling to 0° C. Pyridine sulfur trioxide (4.351 g, 27.3 mmol) was added and the reaction stirred at 0° C. for 1 hour. The reaction was poured into 50 mL water and extracted with ethyl acetate. The combined organics were dried over MgSO₄, then filtered and concentrated in vacuo affording a semi-solid, which was further purified by triturating with 20% ethyl acetate/hexane, affording the desired product as a solid. (1.35 g, 68% yield).

Step F: Preparation of 8-bromo-6-fluoro-2-(2-methoxyvinyl)quinoline: (Methoxymethyl)triphenylphosphonium chloride (1.5 g, 4.3 mmol) was weighed into a 50 mL flask and dissolved in 40 mL of anhydrous THF. The reaction was cooled to 0° C., followed by dropwise addition of KOtBu (4.7 ml, 4.7 mmol). The reaction was allowed to stir for 15 minutes at 23° C., followed by dropwise addition of 8-bromo-6-fluoroquinoline-2-carbaldehyde (1.0 g, 3.9 mmol) as a solution in 10 mL of THF over 3 minutes. The reaction was allowed to stir at ambient temperature for 12 hours. The crude reaction was concentrated in vacuo, followed by trituration with Et₂O, and ethyl acetate, affording the desired product as a solid. (900 mg, 82% yield) MS APCI (+) m/z 282.2 and 284 (M+1 of each isotope) detected.

Step G: Preparation of 8-bromo-6-fluoro-2-(7-(2-methoxyethoxy)-imidazo[1,2-a]pyridin-3-yl)quinoline: 8-Bromo-6-fluoro-2-(2-methoxyvinyl)quinoline (900 mg, 3.19 mmol) was dissolved in a solution of 20 mL of THF and 4 mL of deionized water. N-Bromosuccinimide (596 mg, 3.35 mmol) was added and the reaction monitored by TLC/LC for complete conversion to the alpha-bromo aldehyde. 4-(2-Methoxyethoxy)pyridin-2-amine (537 mg, 3.19 mmol) was added, and the reaction heated to reflux for 10 hours. The crude reaction mixture was concentrated in vacuo affording a solid which was triturated successively with ethyl acetate and Et₂O, followed by trituration with a 1:1 mixture of Et2O and DCM, to afford the desired product as powder (746 mg, 56% yield). MS APCI (+) m/z 416.2 and 418.1 (M+1 of each isotope) detected.

Step H: Preparation of tert-butyl 1-(6-fluoro-2-(7-(2-methoxyethoxy)-imidazo[1,2-a]pyridin-3-yl)quinolin-8-yl)piperidin-4-ylcarbamate: 8-Bromo-6-fluoro-2-(7-(2-methoxyethoxy)H-imidazo[1,2-a]pyridin-3-yl)quinoline (200 mg, 0.48 mmol), tert-butyl piperidin-4-ylcarbamate (125.1 mg, 0.62 mmol) and Cs₂CO₃ (156.6 mg, 0.48 mmol) were weighed into a 5.0 mL reaction vial and suspended in 2.0 ml of anhydrous toluene. The solution was purged with Argon, followed by addition of Pd₂ dba₃ (22.00 mg, 0.02402 mmol) and Binap-rac (29.9 mg, 0.048 mmol). The reaction was heated at 95° C. for 24 hours and then filtered through GF filter paper. The filtrate was washed with 30 mL of DCM and the combined organics were concentrated in vacuo. The crude mixture was purified by flash column chromatography (eluting with a 1-10% MeOH/DCM gradient). The resulting solid was triturated with Et₂O to remove Binap-(OH), followed by a second flash column chromatography purification (eluting with 4% MeOH/DCM), affording the desired product as a foam. (60 mg, 23% yield) MS APCI (+) m/z 536.2 and 537.2 (M+1 of each isotope) detected.

Step I: Preparation of 1-(6-fluoro-2-(7-(2-methoxyethoxy)-imidazo[1,2-a]pyridin-3-yl)quinolin-8-yl)piperidin-4-amine: tert-Butyl 1-(6-fluoro-2-(7-(2-methoxyethoxy)H-imidazo[1,2-a]pyridin-3-yl)quinolin-8-yl)piperidin-4-ylcarbamate (60 mg, 0.11 mmol) was weighed into a 25 mL flask and dissolved in 4.0 mL of DCM, followed by addition of TFA (0.863 ml, 11.2 mmol). The reaction was stirred at ambient temperature for 1 hour, at which time the reaction was complete. The crude reaction was then concentrated in vacuo, followed by triturating 3 times with 10 mL of anhydrous Et₂O, affording the desired product as a solid (37 mg, 76% yield). MS APCI (+) m/z 436.3 (M+1) detected.

### Example 27

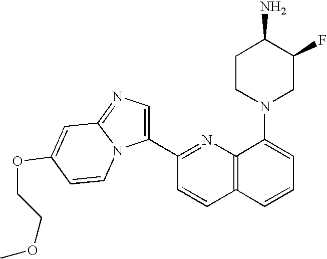

### (cis)-3-fluoro-1-(2-(7-(2-methoxyethoxy)imidazo[1,2-a]pyridin-3-yl)quinolin-8-yl)piperidin-4-amine

Step A: Preparation of (cis)-tert-butyl 4-amino-3-fluoropiperidine-1-carboxylate: Palladium (10.6 g, 4.99 mmol) on carbon (10% Pd, 50% water) and MeOH (150 mL) were added to a 500 mL flask, which was then purged with N₂. (cis)-tert-Butyl 4-(benzylamino)-3-fluoropiperidine-1-carboxylate (15.4 g, 49.9 mmol), and ammonium formate (12.6 g, 200 mmol) was added, and the reaction was heated to reflux for 1 hour. The reaction was cooled to ambient temperature and filtered through Celite (rinsing with CH₂Cl₂). The filtrate was concentrated and the residue was dissolved in CH₂Cl₂ (100 mL), dried over (Na₂SO₄), filtered and condensed to obtain 8.86 g of the title product.

Step B: Preparation of (cis)-tert-butyl 4-(benzyloxycarbonylamino)-3-fluoropiperidine-1-carboxylate: A 125 mL flask was charged with (cis)-tert-butyl 4-amino-3-fluoropiperidine-1-carboxylate (0.512 g, 2.35 mmol), potassium carbonate (0.389 g, 2.82 mmol), benzyl carbonochloridate (0.36 ml, 2.6 mmol), THF (5 mL) and water (1 mL). The reaction was stirred for 12 hours, and then diluted with EtOAc and water. Concentration of the combined organics afforded 923 mg of an oil. Further purification of the oil by filtration through Varian SCX column, eluting with CH₂Cl₂, provided 771 mg of the product as an oil.

Step C: Preparation of benzyl (cis)-3-fluoropiperidin-4-ylcarbamate: 2,2,2-Trifluoroacetic acid (2 ml, 2.19 mmol) was added to a solution of (cis)-tert-butyl 4-(benzyloxycarbonylamino)-3-fluoropiperidine-1-carboxylate (0.771 g, 2.19 mmol) in CH₂Cl₂ (22 mL), and the reaction was stirred for 4 hours. The reaction was diluted with saturated NaHCO₃ and extracted with CH₂Cl₂. The combined organic phases were dried with Na₂SO₄, then filtered and condensed to obtain 538 mg of a thick oil. The oil was then placed under high vacuum for 48 hours, solidifying to a white solid (450 mg).

Step D: Preparation of benzyl (cis)-3-fluoro-1-(2-(7-(2-methoxyethoxy)imidazo[1,2-a]pyridin-3-yl)quinolin-8-yl)piperidin-4-ylcarbamate: 2-(7-(2-methoxyethoxy)imidazo[1,2-a]pyridin-3-yl)quinolin-8-yl trifluoromethanesulfonate (0.200 g, 0.43 mmol), benzyl (cis)-3-fluoropiperidin-4-ylcarbamate (0.141 g, 0.56 mmol), cesium carbonate (0.196 g, 0.60 mmol), Binap-rac (0.021 g, 0.035 mmol), and Pd₂ dba₃, (0.016 g, 0.017 mmol) were weighed into a 25 mL reaction flask and dissolved in 3.0 mL of anhydrous toluene, followed by heating to 115° C. overnight. The crude reaction mixture was cooled to ambient temperature, diluted with CHCl₃, and filtered through GF/F paper. The mother liquor was condensed and purified by flash column chromatography (Horizon-SP1; gradient elution 1-20% MeOH/DCM), affording the desired product as a semi-solid (60%, 147 mg).

Step E: Preparation of (cis)-3-fluoro-1-(2-(7-(2-methoxyethoxy)imidazo[1,2-a]pyridin-3-yl)quinolin-8-yl)piperidin-4-amine: A 25 mL round bottom flask was charged with benzyl (cis)-3-fluoro-1-(2-(7-(2-methoxyethoxy)imidazo[1,2-a]pyridin-3-yl)quinolin-8-yl)piperidin-4-ylcarbamate (0.116 g, 0.21 mmol), dissolved in THF (1 mL), EtOH (1 mL), 2 N HCl (0.5 mL). Pd/C (0.0433 g, 0.041 mmol) was then added, and the reaction placed under a balloon of H₂ and stirred for 24 hours. The Pd/C was removed by filtration and the filtrate was concentrated. Water was added to the resulting crude oil, followed by a DCM wash. The crude aqueous layer was neutralized with saturated NaHCO₃, followed by extraction with CHCl₃, which was then combined, dried over sodium sulfate and condensed, affording 75 mg of the desired product as a solid. The residual oil was then purified by Flash column chromatography on Horizon SP-1 (using a gradient elution of CHCl₃/MeOH/NH₃), affording an additional 48 mg of the desired product. MS APCI (+) m/z 436.3 (M+1) detected.

### Example 28

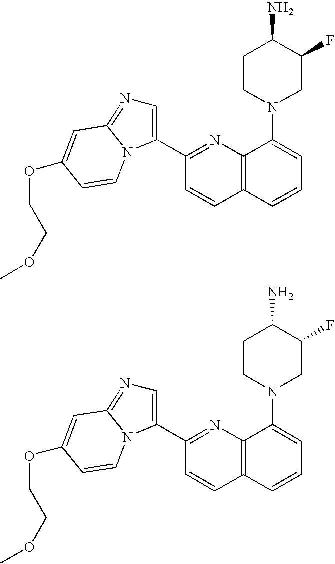

### (3S,4R)-3-fluoro-1-(2-(7-(2-methoxyethoxy)imidazo[1,2-a]pyridin-3-yl)quinolin-8-yl)piperidin-4-amine and (3R,4S)-3-fluoro-1-(2-(7-(2-methoxyethoxy)imidazo[1,2-a]pyridin-3-yl)quinolin-8-yl)piperidin-4-amine

Benzyl (cis)-3-fluoro-1-(2-(7-(2-methoxyethoxy)imidazo[1,2-a]pyridin-3-yl)quinolin-8-yl)piperidin-4-ylcarbamate (prepared as in Example 27) was separated by chiral HPLC (2 cm×250 mm Chiral Technologies OD-H column; mobile phase 6% Methanol, 12% Ethanol, 82% hexanes; flow rate 20 mL/min; 220 nM) to afford a first eluting peak (Peak 1) in 92% ee and a second eluting peak (Peak 2) in 84% ee. Cbz deprotection of each enantiomer led to the title compounds as the HCl salts. MS APCI (+) m/z 436.3 (M+1) detected for both enantiomers.

### Example 29

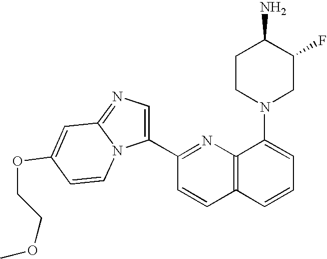

### (trans)-3-fluoro-1-(2-(7-(2-methoxyethoxy)imidazo[1,2-a]pyridin-3-yl)quinolin-8-yl)piperidin-4-amine

Prepared according to Example 27, using benzyl (trans)-3-fluoropiperidin-4-ylcarbamate in place of benzyl (cis)-3-fluoropiperidin-4-ylcarbamate. MS APCI (+) m/z 436.3 (M+1) detected.

### Example 30

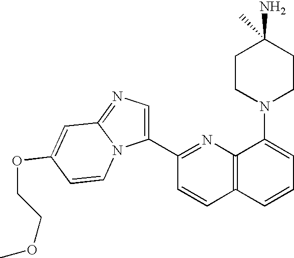

### 1-(2-(7-(2-methoxyethoxy)imidazo[1,2-a]pyridin-3-yl)quinolin-8-yl)-4-methylpiperidin-4-amine

Step A: Preparation of 1-tert-butyl 4-ethyl piperidine-1,4-dicarboxylate: The compound was prepared following a procedure outlined in PCT Publication No. WO 01/40217. Ethyl piperidine-4-carboxylate (8.639 ml, 56.10 mmol) was dissolved in dichloromethane (55 mL) and treated in three equal portions with di-tert-butyl dicarbonate (12.24 g, 56.10 mmol). Each addition caused vigorous bubbling and a bit of temperature rise. After the additions, the solution was stirred at ambient temperature for 14 hours. The solution was extracted three times with saturated NaHCO₃, dried over Na₂SO₄ and concentrated in vacuo to provide the desired product as an oil (14.1 g). ¹H NMR (400 MHz, CDCl₃) δ 4.14 (q, 2H), 4.09-3.95 (brd, 2H), 2.90-2.78 (m, 2H), 2.49-2.38 (m, 1H), 1.92-1.82 (m, 2H), 1.69-1.57 (m, 2H), 1.46 (s, 9H), 1.26 (t, 3H).

Step B: Preparation of 1-tert-butyl 4-ethyl 4-methylpiperidine-1,4-dicarboxylate: The compound was prepared following a procedure outlined in PCT Publication No. WO 01/40217. 1-tert-Butyl 4-ethyl piperidine-1,4-dicarboxylate (7.12 g, 27.7 mmol) was dissolved in THF (30 mL) and cooled to −40° C. LHMDS (55.3 ml, 55.3 mmol) was added slowly and the solution was stirred at −40° C. for 1 hour. Iodomethane (3.45 ml, 55.3 mmol) was added and the reaction mixture was warmed to ambient temperature and stirred for 14 hours. The reaction was quenched with water and saturated NaHCO₃. After diluting with methylene chloride, the layers were separated. The aqueous layer was washed twice with methylene chloride, and the combined organic layers were washed with saturated NaCl, dried over Na₂SO₄ and concentrated in vacuo to provide the desired product as an oil (quantitative). ¹H NMR (400 MHz, CDCl₃) δ 4.16 (q, 2H), 3.83-3.70 (m, 2H), 3.03-2.94 (m, 2H), 2.11-2.02 (m, 2H), 1.45 (s, 9H), 1.41-1.30 (m, 2H), 1.26 (t, 3H), 1.20 (s, 3H).

Step C: Preparation of 1-(tert-butoxycarbonyl)-4-methylpiperidine-4-carboxylic acid: The compound was prepared following a procedure outlined in PCT Publication No. WO 01/40217. 1-tert-Butyl 4-methylpiperidine-1,4-dicarboxylate (54.2 g, 200 mmol) was dissolved in a solution of EtOH (400 mL) and 2N NaOH (200 mL). The mixture was heated to 60° C. for 60 hours then cooled and concentrated in vacuo. The solution was extracted three times with Et₂O, and the aqueous layer was adjusted to pH 3 with a mixture of concentrated HCl followed by 3N HCl. The aqueous was extracted three times with ethyl acetate then the combined organic layers were washed with saturated NaCl, dried over Na₂SO₄ and concentrated in vacuo to provide the desired product as a solid (45.1 g). ¹H NMR (400 MHz, CDCl₃) δ 3.86-3.67 (brd m, 2H), 3.13-3.01 (m, 2H), 2.12-2.01 (m, 2H), 1.53-1.32 (m, 2H), 1.45 (s, 9H), 1.27 (s, 3H).

Step D: Preparation of benzyl 4-methylpiperidin-4-ylcarbamate: The compound was prepared following a procedure outlined in Madar, D. J.; et al.; J. Med. Chem. 2006, 49, 6416-6420, and supplementary materials. 1-(tert-Butoxycarbonyl)-4-methylpiperidine-4-carboxylic acid (5.00 g, 20.5 mmol) was dissolved in toluene (40 mL) was treated at ambient temperature with triethylamine (4.30 ml, 30.8 mmol) and diphenylphosphoryl azide (5.98 ml, 27.7 mmol). The reaction was stirred at ambient temperature for 45 minutes then phenylmethanol (10.6 ml, 102 mmol) was added and the mixture was heated to 80° C. for 16 hours. The reaction mixture was concentrated in vacuo. The residue was redissolved in ethyl acetate and washed three times with saturated NH₄Cl and once with saturated NaCl. The organic layer was dried over Na₂SO₄ and concentrated in vacuo to provide the desired product as a semi-solid (25 g), which was utilized in the next step without purification.

Step E: Preparation of benzyl 4-methylpiperidin-4-ylcarbamate: tert-Butyl 4-(benzyloxycarbonylamino)-4-methylpiperidine-1-carboxylate (2.38 g, 6.83 mmol) was dissolved in MeOH (10 mL) and treated with 4 M hydrogen chloride in dioxane (25.6 ml, 102 mmol). The solution was stirred at ambient temperature for 14 hours then concentrated in vacuo. The residue was redissolved in methylene chloride and adjusted to pH 10 with 15% NaOH. The layers were separated and the aqueous was washed twice with methylene chloride. The combined organic layers were dried over Na₂SO₄ and concentrated in vacuo. The residue was dissolved in methylene chloride (20 mL) and applied to a pre-equilibrated (methylene chloride) Varian Bond Elut SCX column (10 g). The column was eluted sequentially under slightly reduced pressure with 150 mL fractions of methylene chloride, 10% MeOH in methylene chloride, 20% (6% NH₄OH in MeOH) in methylene chloride. The final fraction was concentrated in vacuo then redissolved in methylene chloride, dried over Na₂SO₄ and concentrated in vacuo to provide the desired product (1.54 g). ¹H NMR (400 MHz, CDCl₃) δ 7.42-7.29 (m, 5H), 5.06 (s, 2H), 4.67-4.58 (brd s, 1H), 2.85-2.79 (m, 4H), 1.94-1.89 (brd m, 2H), 1.61-1.51 (m, 2H), 1.38 (s, 3H). MS APCI (+) m/z 249.0 (M+1) detected.

Step F: Preparation of benzyl 1-(2-(7-(2-methoxyethoxy)imidazo[1,2-a]pyridin-3-yl)quinolin-8-yl)-4-methylpiperidin-4-ylcarbamate: Benzyl 4-methylpiperidin-4-ylcarbamate (3.46 g, 13.9 mmol), 2-(7-(2-methoxyethoxy)imidazo[1,2-a]pyridin-3-yl)quinolin-8-yl trifluoromethanesulfonate (5.01 g, 10.7 mmol), micronized Cs₂CO₃ (4.89 g, 15.0 mmol), BINAP-racemic (1.33 g, 2.14 mmol) and Pd₂ dba₃ (0.981 g, 1.07 mmol) were combined in toluene (70 mL). The solution was degassed with argon then heated to reflux under argon for 14 hours. The reaction was cooled, diluted with CHCl₃ and filtered through GF/F paper. The filtered solids were washed with CHCl₃ and the filtrate was concentrated in vacuo. The crude material was purified by chromatography on SiO₂, eluting with a gradient from 1%-20% (6% NH₄OH in MeOH) in ethyl acetate, (3.24 g). ¹H NMR (400 MHz, CDCl₃) δ 10.37 (d, J=7.7 Hz, 1H), 8.22 (s, 1H), 8.08 (d, J=8.6 Hz, 1H), 7.81 (d, J=8.5 Hz, 1H), 7.46-7.29 (m, 5H), 7.28-7.23 (m, 1H), 7.02 (d, J=2.5 Hz, 1H), 6.85-6.79 (brd m, 1H), 5.11 (brd s, 2H), 4.76 (brd s, 1H), 4.27-4.21 (m, 2H), 3.86-3.81 (m, 2H), 3.61-3.52 (m, 2H), 3.49 (s, 3H), 3.22-3.08 (m, 2H), 2.40-2.30 (brd m, 2H), 2.11-1.97 (m, 2H), 1.56 (s, 3H). MS APCI (+) m/z 566.2 (M+1) detected.

Step G: Preparation of 1-(2-(7-(2-methoxyethoxy)imidazo[1,2-a]pyridin-3-yl)Quinolin-8-yl)-4-methylpiperidin-4-amine: Benzyl 1-(2-(7-(2-methoxyethoxy)imidazo[1,2-a]pyridin-3-yl)quinolin-8-yl)-4-methylpiperidin-4-ylcarbamate (3.95 g, 6.98 mmol) and 20% Pd(OH)₂ on carbon (2.94 g, 4.19 mmol) were slurried in THF:EtOH (1:1, 86 mL) and the suspension was treated with concentrated HCl (53 drops). The reaction was placed under a hydrogen atmosphere (balloon) and stirred overnight at ambient temperature. The reaction was diluted with MeOH to dissolve a small amount of precipitated solids. The catalyst was removed by filtration, and the filtrate was concentrated in vacuo. The residue was redissolved in MeOH and treated with a solution of 7 N NH₃ in MeOH (10 mL). The mixture was re-concentrated in vacuo. The residue was purified by silica gel chromatography, eluting with a gradient from 1-20% (6% NH₄OH in MeOH) in methylene chloride, (0.96 g). The free base (0.96 g, 2.2 mmol) was dissolved in MeOH (2 mL) and treated with a solution of 4M HCl in dioxane (4.5 mL). The mixture was concentrated in vacuo four times from MeOH. The residue was dissolved in MeOH (40 mL) and slowly added dropwise to a stirred flask containing Et₂O (1600 mL). The resultant suspension was stirred for 30 minutes. The solids were collected by filtration, washed with Et₂O and dried under a blanket of nitrogen gas to provide the desired product as a solid (0.91 g). ¹H NMR (400 MHz, CD₃OD) δ 10.49 (d, J=7.9 Hz, 1H), 8.80 (s, 1H), 8.57 (d, J=9.1 Hz, 1H), 8.19 (d, J=8.5 Hz, 1H), 8.02-7.87 (m, 2H), 7.74 (t, J=7.8 Hz, 1H), 7.68-7.62 (dd, 1H), 7.44 (s, 1H), 4.50-4.44 (m, 2H), 4.08-3.99 (m, 2H), 3.90-3.84 (m, 2H), 3.75-3.62 (m, 2H), 3.45 (s, 3H), 2.61-2.47 (m, 2H), 2.30-2.19 (m, 2H), 1.63 (s, 3H). MS APCI (+) m/z 432.1 (M+1) detected.

### Example 31

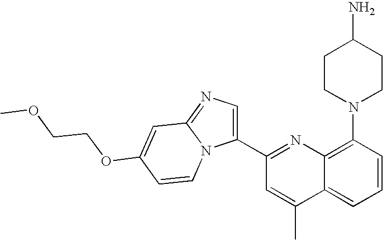

### 1-(2-(7-(2-methoxyethoxy)imidazo[1,2-a]pyridin-3-yl)-4-methylquinolin-8-yl)piperidin-4-amine

Step A: Preparation of N-(2-bromophenyl)-3-oxobutanamide: 2-Bromobenzenamine (10.00 g, 58.13 mmol) and ethyl 3-oxobutanoate (14.72 ml, 116.3 mmol) were weighed into a 100 mL flask and heated to reflux overnight. The reaction was cooled and concentrated in vacuo. The crude material was purified by flash column chromatography (eluting with a 0-5% MeOH/DCM gradient), affording the desired product as a white solid (2.7 g, 19% yield) MS APCI (−) m/z 254.0 and 255.9 (M−1 of each isotope) detected.

Step B: Preparation of 8-bromo-4-methylquinolin-2(1H)-one: N-(2-bromophenyl)-3-oxobutanamide (2.0 g, 7.81 mmol) was, dissolved in 10 mL of sulfuric acid and heated to 95° C. for 1 hour. The crude mixture was cooled to ambient temperature and poured onto 30 mL of water. The aqueous was extracted with ethyl acetate, and the combined organic layers were dried over Na₂SO₄, filtered, and concentrated to afford the desired product as a solid. (1.47 g, 79% yield) MS APCI (+) m/z 238.4 and 240.2 (M+1 of each isotope) detected.

Step C: Preparation of 2,8-dibromo-4-methylquinoline: 8-Bromo-4-methylquinolin-2(1H)-one (300 mg, 1.26 mmol) was melted into phosphorous oxybromide (3.411 g, 12.6 mmol) and heated gently from 75° C. to 150° C., followed by heating for two hours at 150° C. The reaction was cooled to 60° C., and then poured into 20 mL of ice water, affording the desired product as a precipitate which was collected by filtration (320 mg, 84% yield), MS APCI (+) m/z 300.3, 302.2 and 304.2 (M+1 of each isotope) detected.

Step D: Preparation of 8-bromo-2-(7-(2-methoxyethoxy)-imidazo[1,2-a]pyridin-3-yl)-4-methylquinoline: 2,8-Dibromo-4-methylquinoline (300 mg, 0.99 mmol), 7-(2-methoxyethoxy)H-imidazo[1,2-a]pyridine (192 mg, 0.99 mmol), Pd(PPh₃)₄ (57.6 mg, 0.050 mmol), K₂CO₃ (276 mg, 2 mmol), and Pd(OAc)₂ (11.2 mg, 0.050 mmol) were weighed into dioxane (3.99 ml) and water (0.039 ml) and the reaction mixture was heated to 100° C. for 12 hours. The reaction was cooled, then concentrated in vacuo, and the residue was triturated with 1:1 ethyl acetate/MTBE. The mother liquor was purified by flash column chromatography (1-15% MeOH/DCM gradient elution), affording the desired product as a solid (247 mg, 60.1% yield). MS APCI (+) m/z 412.2 and 414.2 (M+1 of each isotope) detected.

Step E: Preparation of 1-(2-(7-(2-methoxyethoxy)H-imidazo[1,2-a]pyridin-3-yl)-4-methylquinolin-8-yl)piperidin-4-ylcarbamate: 8-Bromo-2-(7-(2-methoxyethoxy)H-imidazo[1,2-a]pyridin-3-yl)-4-methylquinoline (100 mg, 0.242 mmol), tert-butyl piperidin-4-ylcarbamate (63 mg, 0.315 mmol) and Cs₂CO₃ (79 mg, 0.242 mmol) were weighed into a 5.0 mL reaction vial and suspended in 2.0 ml of anhydrous toluene. The solution was purged with Argon followed by addition of Pd₂ dba₃ (11 mg, 0.012 mmol), Binap-rac (15 mg, 0.024 mmol). The reaction was heated at 95° C. for about 1.5 days. The reaction was cooled to ambient temperature, followed by filtration through a Celite® plug to remove catalyst. The crude reaction was concentrated in vacuo and purified by flash column chromatography (1-10% MeOH/DCM gradient elution), affording the desired product as a solid (79 mg, 61%). MS APCI (+) m/z 532.2 and 533.3 (M+1 of each isotope) detected.

Step F: Preparation of 1-(2-(7-(2-methoxyethoxy)imidazo[1,2-a]pyridin-3-yl)-4-methylquinolin-8-yl)piperidin-4-amine: Preparation of trifluoroacetic acid salt: tert-butyl 1-(2-(7-(2-methoxyethoxy)H-imidazo[1,2-a]pyridin-3-yl)-4-methylquinolin-8-yl)piperidin-4-ylcarbamate (50 mg, 0.094 mmol) was weighed into a 25 mL flask and dissolved in 5.0 mL of DCM followed by addition of TFA (0.725 ml, 9.4 mmol). The reaction was stirred under N₂ for 35 minutes. The reaction was concentrated and the residue was triturated with 10 mL of anhydrous Et₂O, affording the desired product as a yellow solid. (31 mg, 60% yield) MS APCI (+) m/z 432.3 and 433.2 (M+1 of each isotope) detected.

### Example 32

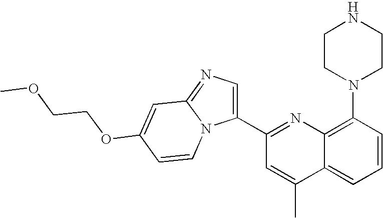

### 2-(7-(2-methoxyethoxy)imidazo[1,2-a]pyridin-3-yl)-4-methyl-8-(piperazin-1-yl)quinoline

Prepared according to Example 31, using tert-butyl piperazine-1-carboxylate in place of tert-butyl piperidin-4-ylcarbamate. MS APCI (+) m/z 418.4 (M+1) detected.

### Example 33

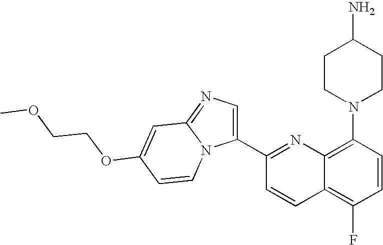

### 1-(5-fluoro-2-(7-(2-methoxyethoxy)imidazo[1,2-a]pyridin-3-yl)quinolin-8-yl)piperidin-4-amine

Step A: Preparation of 8-bromo-5-fluoro-2-methylquinoline: 2-Bromo-5-fluorobenzenamine (15 g, 78.94 mmol) was weighed into a 100 mL flask and dissolved in 100 mL of 6N HCl. The reaction mixture was heated to reflux, followed by dropwise addition of (E)-but-2-enal (6.87 ml, 83 mmol) mixed with 1.0 mL deionized water over 25 minutes. Following complete addition, the reaction was heated at 100° C. for an additional 35 minutes. The reaction was cooled to ambient temperature, followed by addition of 50 mL of Et₂O. The reaction was stirred for 5 minutes followed by removal of Et₂O by separatory funnel. ZnCl₂ (3.587 g, 26 mmol) was added to the aqueous layer in two portions and the reaction mixture was cooled to 0° C. over 30 minutes. The aqueous layer was then taken to pH 8.0 using concentrated NH₄OH. The aqueous layer was then extracted with Et₂O and then EtOAc. The combined organic phases were dried over Na₂SO₄, filtered and concentrated in vacuo, affording the desired product 8-bromo-5-fluoro-2-methylquinoline (18.1 g, 95% yield) as a solid.

Step B: Preparation of 8-bromo-2-(dibromomethyl)-5-fluoroquinoline: 8-Bromo-5-fluoro-2-methylquinoline (18.1 g, 75.4 mmol) was weighed into a 1000 mL flask, followed by addition of NaOAc (37.1 g, 452 mmol). The solids were suspended in 500 mL of AcOH, and the reaction heated to 70° C. Bromine (11.6 ml, 226 mmol) was added dropwise over 25 minutes as a solution in 50 mL of AcOH. Following complete addition, the reaction was stirred at 100° C. for 1 hour. The reaction was then cooled to ambient temperature, then poured onto 700 cc of ice. The ice was allowed to melt completely and the mixture was extracted with ethyl acetate. The combined organic phases were dried over Na₂SO₄, filtered, concentrated in vacuo and dried on high-vacuum over night, affording the desired product (27 g, 90%).

Step C: Preparation of 8-bromo-5-fluoroquinoline-2-carboxylate and 8-bromo-5-fluoroquinoline-2-carboxylic acid: 8-Bromo-2-(dibromomethyl)-5-fluoroquinoline (25 g, 63 mmol) was weighed into a 1000 mL 1 flask and dissolved in 250 mL of EtOH, followed by addition of silver nitrate (34 g, 201 mmol) in 100 mL of 1:1 EtOH/H₂O. The reaction was heated to reflux for 1 hour, then filtered hot through a medium frit sintered glass funnel, affording 2169 mg of a powder. The mother liquor was concentrated in vacuo, followed by extractive work-up (500 mL EtOAc/water). The combined organic phases were dried over Na₂SO₄ and concentrated in vacuo to afford ethyl 8-bromo-5-fluoroquinoline-2-carboxylate (9.3 g, 99% yield) and 8-bromo-5-fluoroquinoline-2-carboxylic acid (8 g, 94% yield).

Step D: Preparation of (8-bromo-5-fluoroquinolin-2-yl)methanol: Ethyl 8-bromo-5-fluoroquinoline-2-carboxylate (5.52 g, 18.5 mmol) was weighed into 1000 mL flask and dissolved in 400 mL of DCM. The reaction was cooled to −78° C., followed by dropwise addition of DIBAL-H (49.4 ml, 74.1 mmol) over 10 minutes. The reaction was allowed to stir and warm to ambient temperature over 2 hours. The reaction was then quenched with 10 mL MeOH and 100 mL of Rochelle's salts, followed by stirring overnight. The aqueous layer was extracted with EtOAc, followed by concentration in vacuo. The residue was purified by flash column chromatography (20-50% EtOAc/Hex), affording the desired product as a solid (2.25 g, 24% yield). MS APCI (+) m/z 256.1 (M+1) detected.

Step E: Preparation of 8-bromo-5-fluoroquinoline-2-carbaldehyde: (8-Bromo-5-fluoroquinolin-2-yl)methanol (1.85 g, 7.22 mmol), DMSO (8.20 ml, 116 mmol) and triethylamine (4.53 ml, 32.5 mmol) were weighed into a 100 mL flask and dissolved in a 1:1 mixture of DCM/DMSO, followed by cooling to 0° C. Pyridine sulfur trioxide (4.02 g, 25.3 mmol) was added and the reaction was stirred at 0° C. for 1 hour. The reaction was poured into 50 mL water and extracted with EtOAc. The combined organic phases were then dried over MgSO₄, filtered, and concentrated in vacuo affording a semi-solid, which was further purified by flash column chromatography 10-40% EtOAc/Hexane, affording 8-bromo-5-fluoroquinoline-2-carbaldehyde (1.71 g, 93% yield).

Step F: Preparation of 8-bromo-5-fluoro-2-(2-methoxyvinyl)quinoline: (methoxymethyl)triphenylphosphonium chloride (1.9 g, 5.6 mmol) was weighed into a 100 mL 1 neck round bottom flask and dissolved in 40 mL of anhydrous THF. The reaction was then cooled to 0° C., followed by dropwise addition of KOtBu (6.1 ml, 6.1 mmol). The reaction was stirred for 15 minutes at ambient temperature, followed by dropwise addition of 8-bromo-5-fluoroquinoline-2-carbaldehyde (1.3 g, 5.1 mmol) as a solution in 10 mL of THF over 3 minutes, affording an immediate dark red/brown color change. The reaction was allowed to stir at ambient temperature for 12 hours. The reaction was concentrated in vacuo, followed by trituration with Et₂O, then ethyl acetate, to afford the crude desired product, which was used directly in the subsequent step.

Step G: Preparation of 8-bromo-5-fluoro-2-(7-(2-methoxyethoxy)-imidazo[1,2-a]pyridin-3-yl)quinoline: 8-Bromo-5-fluoro-2-(2-methoxyvinyl)quinoline (2.4 g, 8.5 mmol) was dissolved in a solution of 20 mL of THF and 4 mL of deionized water. N-Bromosuccinimide (1.59 g, 8.9 mmol) was added and the reaction was stirred for 2 hours. 4-(2-Methoxyethoxy)pyridin-2-amine (1.43 g, 8.51 mmol) was added, and the reaction was heated to reflux for 10 hours. The crude reaction mixture was concentrated in vacuo affording a solid which was triturated successively with EtOAc and Et₂O, followed by trituration with a mixture of Et₂O and CH₂Cl₂ to afford 8-bromo-5-fluoro-2-(7-(2-methoxyethoxy)imidazo[1,2-a]pyridin-3-yl)quinoline (746 mg, 21% yield) as a solid.

Step H: Preparation of tert-butyl 1-(6-fluoro-2-(7-(2-methoxyethoxy)-imidazo[1,2-a]pyridin-3-yl)quinolin-8-yl)piperidin-4-ylcarbamate: 8-Bromo-5-fluoro-2-(7-(2-methoxyethoxy)imidazo[1,2-a]pyridin-3-yl)quinoline (291 mg, 0.70 mmol), tert-butyl piperidin-4-ylcarbamate (182 mg, 0.91 mmol) and Cs₂CO₃ (319 mg, 0.98 mmol) were weighed into a 25 mL reaction flask and suspended in 10.0 ml of anhydrous toluene. The solution was purged with Argon followed by addition of Pd₂ dba₃ (32 mg, 0.035 mmol) and Binap-rac (31 mg, 0.05 mmol). The reaction was heated at 95° C. for 24 hours. The reaction was filtered through GF paper, and the filtrate was washed with DCM and concentrated in vacuo. The residue was purified by flash column chromatography (1-10% MeOH/CH₂Cl₂). The resulting solid was triturated with Et₂O to remove Binap-(OH), affording the desired product (300 mg, 80%) as an oil. MS APCI (+) m/z 536.2 (M+1) detected.

Step I: Preparation of 1-(6-fluoro-2-(7-(2-methoxyethoxy)-imidazo[1,2-a]pyridin-3-yl)quinolin-8-yl)piperidin-4-amine: tert-Butyl 1-(5-fluoro-2-(7-(2-methoxyethoxy)imidazo[1,2-a]pyridin-3-yl)quinolin-8-yl)piperidin-4-ylcarbamate (300 mg, 0.56 mmol) was weighed into a 25 mL flask and dissolved in 40.0 mL of CH₂Cl₂, followed by addition of TFA (4.3 ml, 56.0 mmol). The reaction was stirred at ambient temperature for 1 hour, then concentrated in vacuo. The residue was purified by flash column chromatography (1-20% MeOH/CH₂Cl₂), affording the desired product (30 mg, 12%) as a solid. MS APCI (+) m/z 436.2 (M+1) detected.

### Example 34

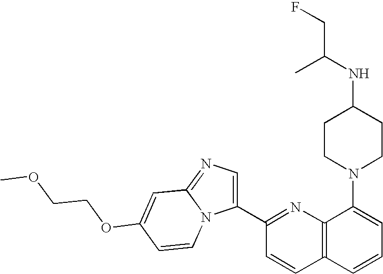

### N-(1-fluoropropan-2-yl)-1-(2-(7-(2-methoxyethoxy)imidazo[1,2-a]pyridine-3-yl)quinolin-8-yl)piperidin-4-amine

Fluoroacetone (3.2 mg, 3.0 4, 0.04 mmol) was added via syringe to a solution of 1-(2-(7-(2-methoxyethoxy)imidazo[1,2-a]pyridine-3-yl)quinolin-8-yl)piperidin-4-amine (Example 2; 22.6 mg, 0.05 mmol) and triethylamine (4.2 mg, 5.8 μL, 0.04 mmol) in 0.4 ml of a 1:1 MeOH:THF mixture. The reaction mixture was stirred at ambient temperature under a nitrogen atmosphere for 3 hours. Sodium tetrahydroborate (3 mg, 0.08 mmol) was added, and the reaction mixture stirred at ambient temperature overnight. The reaction mixture was concentrated, the residue was suspended in 5 ml saturated aqueous sodium bicarbonate solution, and the resulting mixture was extracted with 10 ml of chloroform. The organic layer was dried over Na₂SO₄, filtered and concentrated under reduced pressure to produce an oily solid. The solid was purified by silica gel chromatography (eluting with 10% MeOH-chloroform) to afford the title compound (5.9 mg, 30% yield). MS APCI (+) m/z 478.2 (M+1) detected.

### Example 35

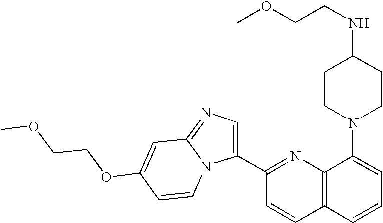

### 1-(2-(7-(2-methoxyethoxy)imidazo[1,2-a]pyridine-3-yl)quinolin-8-yl)-N-(2-methoxyethyl)piperidin-4-amine

Step A: Preparation of benzyl 4-(2-methoxyethylamino)piperidine-1-carboxylate: A solution of 2-methoxyethanamine (0.241 g, 3.22 mmol) and benzyl 4-oxopiperidine-1-carboxylate (0.500 g, 2.14 mmol) in 10 ml of a 1:1 MeOH/THF mixture was stirred at ambient temperature under a nitrogen atmosphere for 1.5 hours. Sodium tetrahydroborate (243 mg, 6.42 mmol) was added, and the resulting mixture was stirred at ambient temperature for 48 hours. The reaction mixture was carefully diluted with saturated aqueous sodium carbonate solution (40 ml) and extracted thoroughly with DCM and EtOAc. The combined organic extracts were dried over anhydrous sodium sulfate, filtered and concentrated to afford an oil (0.56 g, 89% yield) which was used directly in the next step.

Step B: Preparation of N-(2-methoxyethyl)piperidin-4-amine: A solution of benzyl 4-(2-methoxyethylamino)piperidine-1-carboxylate (0.56 g, 1.9 mmol) in absolute ethanol (6 ml) was treated under a nitrogen atmosphere with Pd/C (10% wt, 0.204 g). The reaction flask was flushed with hydrogen. The reaction mixture was stirred at ambient temperature under a hydrogen atmosphere overnight. The reaction mixture was filtered through a Celite pad, and the solids remaining on the pad were rinsed with 30 ml ethanol. The combined filtrates were concentrated, and the residue was dissolved in chloroform, dried over anhydrous sodium sulfate, and concentrated to afford an oil (0.195 g, 64% yield) which was used directly in the next step.

Step C: Preparation of 1-(2-(7-(2-methoxyethoxy)imidazo[1,2-a]pyridine-3-yl)quinolin-8-yl)-N-(2-methoxyethyl)piperidin-4-amine: Prepared from 8-bromo-2-(7-(2-methoxyethoxy)imidazo[1,2-a]pyridin-3-yl)quinoline according to the procedure for Example 1 (step E) using N-(2-methoxyethyl)piperidin-4-amine in place of tert-butyl piperazine-1-carboxylate. MS APCI (+) m/z 476.2 (M+1) detected.

### Example 36

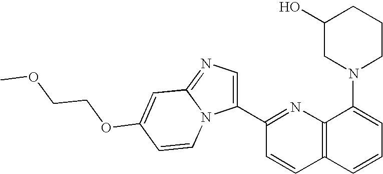

### 1-(2-(7-(2-methoxyethoxy)imidazo[1,2-a]pyridine-3-yl)quinolin-8-yl)piperidin-3-ol

A suspension of cesium carbonate (123 mg, 0.38 mmol), piperidin-3-ol (15.2 mg, 0.15 mmol), tris(dibenzylideneacetone)dipalladium(0) (3.5 mg, 0.0038 mmol), 2,2′-bis(diphenylphosphino)-1,1′-binaphthyl (4.7 mg, 0.0075 mmol), and 8-bromo-2-(7-(2-methoxyethoxy)imidazo[1,2-a]pyridine-3-yl)quinoline (30.0 mg, 0.075 mmol) in anhydrous toluene (3 ml) was degassed thoroughly under a nitrogen atmosphere, then heated at 100° C. for 16 hours. The reaction mixture was poured in water (20 ml), and the resulting mixture was extracted with chloroform and EtOAc. The combined organic extracts were dried over anhydrous sodium sulfate, filtered and concentrated to afford a solid. Chromatography of the crude product on silica gel eluting with MeOH-chloroform yielded 10.3 mg of the title compound (10.3 mg, 33%) as a solid. MS APCI (+) m/z 419.3 (M+1) detected.

### Example 37

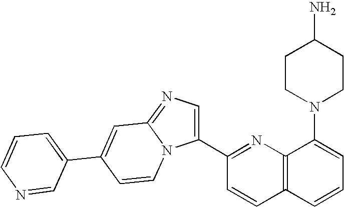

### 1-(2-(7-(pyridine-3-yl)imidazo[1,2-a]pyridine-3-yl)quinolin-8-yl)piperidin-4-amine

Step A: Preparation of 7-bromoimidazo[1,2-a]pyridine: A solution of 4-bromopyridin-2-amine (1.00 g, 5.78 mmol) and 2-chloroacetaldehyde (50% wt aqueous solution, 1.83 ml, 14.45 mmol) in absolute ethanol (9.5 ml) was refluxed for 12 hours, and then allowed to cool to ambient temperature overnight. The reaction mixture was concentrated under reduced pressure and carefully re-suspended in saturated aqueous bicarbonate solution (100 ml). The resulting mixture was extracted thoroughly with DCM and EtOAc, and the combined organic extracts were dried over anhydrous sodium sulfate and concentrated to afford 1.31 g of a solid. The solid was purified by silica gel chromatography (eluting with 3% MeOH-chloroform) to afford the desired compound (0.808 g, 71% yield). MS APCI (+) m/z 197.1 and 199.1 (M+1 for each isotope) detected.

Step B: Preparation of 7-(pyridine-3-yl)imidazo[1,2-a]pyridine: A suspension of potassium carbonate (0.351 g, 2.54 mmol), pyridine-3-ylboronic acid (68.6 mg, 0.558 mmol), 7-bromoimidazo[1,2-a]pyridine (0.100 g, 0.508 mmol) and tetrakis(triphenylphosphine) palladium (0) (29.3 mg, 0.025 mmol) in 6.5 ml of a 1:1:4.5 mixture of water:dimethylformamide:acetonitrile was degassed thoroughly under a nitrogen atmosphere, and heated at 60° C. for 18 hours. The reaction mixture was poured in water (50 ml) and extracted with dichloromethane and EtOAc. The combined organic extracts were dried over anhydrous sodium sulfate and concentrated to afford a solid. The solid was purified by silica gel chromatography (eluting with 6% MeOH-chloroform) to afford the desired compound (74.1 mg, 75% yield). MS APCI (+) m/z 196.3 (M+1) detected.

Step C: Preparation of 8-bromo-2-(7-(pyridine-3-yl)imidazo[1,2-a]pyridine-3-yl)quinoline: A suspension of potassium carbonate (198 mg, 1.43 mmol), palladium(II)acetate (8.1 mg, 0.036 mmol), tetrakis(triphenylphosphine)palladium (41.4 mg, 0.036 mmol), 2,8-dibromoquinoline (206 mg, 0.717 mmol) and 7-(pyridine-3-yl)imidazo[1,2-a]pyridine (140 mg, 0.717 mmol) in 3.03 ml of a 100:1 dioxane:water mixture was degassed under a nitrogen atmosphere. The reaction mixture was heated at 100° C. for 17 hrs. The reaction mixture was concentrated under reduced pressure, resuspended in a small amount of DCM, and the precipitate was isolated by suction filtration through a medium-sized pore sintered glass filter. The precipitate was washed extensively with water, rinsed with a small amount of cold chloroform and MeOH, and dried under high vacuum to yield the desired compound (0.130 g, 45% yield). MS APCI (+) m/z 401.5 and 403.4 (M+1 for each isotope) detected

Step D: Preparation of tert-butyl 1-(2-(7-(pyridine-3-yl)imidazo[1,2-a]pyridine-3-yl)quinolin-8-yl)piperidin-4-ylcarbamate: Prepared according to the procedure for Example 3 using tert-butyl piperidin-4-ylcarbamate in place of piperidin-3-ol, and 8-bromo-2-(7-(pyridine-3-yl)imidazo[1,2-a]pyridine-3-yl)quinoline in place of 8-bromo-2-(7-(2-methoxyethoxy)imidazo[1,2-a]pyridine-3-yl)quinoline. MS APCI (+) m/z 521.1 (M+1) detected.

Step E: Preparation of 1-(2-(7-(pyridine-3-yl)imidazo[1,2-a]pyridine-3-yl)quinolin-8-yl)piperidin-4-amine: A solution of tert-butyl 1-(2-(7-(pyridine-3-yl)imidazo[1,2-a]pyridine-3-yl)quinolin-8-yl)piperidin-4-ylcarbamate (46.3 mg, 0.089 mmol) in 3 ml of a 1:2 chloroform:DCM mixture was treated at ambient temperature with trifluoroacetic acid (0.37 g, 3.24 mmol). The reaction mixture was stirred at ambient temperature for 3 hours, and concentrated to dryness to afford a solid. The solid was dissolved in a small amount of chloroform containing 7 N ammonia in MeOH solution, and the resulting solution was purified by silica gel chromatography (eluting with 10% MeOH-chloroform, then with 7% MeOH (containing 3% 7 N NH₃)/MeOH-chloroform) to yield the title compound (26.8 mg, 72% yield). MS APCI (+) m/z 421.2 (M+1) detected.

### Example 38

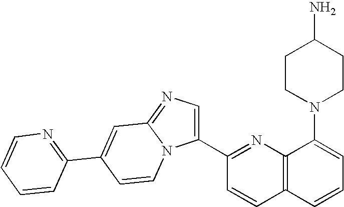

### 1-(2-(7-(pyridine-2-yl)imidazo[1,2-a]pyridine-3-yl)quinolin-8-yl)piperidin-4-amine

Step A: Preparation of 7-(pyridine-2-yl)imidazo[1,2-a]pyridine: A solution of 7-bromoimidazo[1,2-a]pyridine (0.100 g, 0.508 mmol), tri-o-tolylphosphine, tris(dibenzylideneacetone)dipalladium(0) (47 mg, 0.051 mmol), and 2-tri-n-butylstannylpyridine (0.234 g, 0.508 mmol) in anhydrous DMF (5 ml) was combined at ambient temperature under a nitrogen atmosphere with triethylamine (65 mg, 0.65 mmol). The reaction mixture was heated at 100° C. for 17 hours. The reaction mixture was then allowed to cool, poured in water (40 ml), and extracted with DCM, ether, and EtOAc. The combined organic extracts were dried over anhydrous sodium sulfate and concentrated to afford a solid. The solid was purified by silica gel chromatography (eluting with 5% MeOH-DCM) to afford the desired compound (38.4 mg, 39% yield). MS APCI (+) m/z 196.3 (M+1) detected

Steps B-D: Preparation of 1-(2-(7-(Pyridine-2-yl)imidazo[1,2-a]pyridine-3-yl)quinolin-8-yl)piperidin-4-amine: Prepared according to the procedure for Example 37 using 7-(pyridine-2-yl)imidazo[1,2-a]pyridine in place of 7-(pyridine-3-yl)imidazo[1,2-a]pyridine. MS APCI (+) m/z 421.2 (M+1) detected.

### Example 39

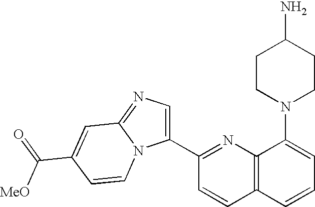

### Methyl 3-(8-(4-aminopiperidin-1-yl)quinolin-2-yl)imidazo[1,2-a]pyridine-7-carboxylate

Step A: Preparation of methyl imidazo[1,2-a]pyridine-7-carboxylate: Prepared according to Example 37, Step A using methyl 2-aminoisonicotinate instead of 4-bromopyridin-2-amine. MS APCI (+) m/z 177.2 (M+1) detected.

Step B: Preparation of methyl 3-(8-bromoquinolin-2-yl)imidazo[1,2-a]pyridine-7-carboxylate: Prepared according to Example 37, Step C using methyl imidazo[1,2-a]pyridine-7-carboxylate instead of 7-(pyridine-3-yl)imidazo[1,2-a]pyridine. MS APCI (+) m/z 382.4 and 384.3 (M+1 for each isotope) detected.

Step C: Preparation of methyl 3-(8-(4-(tert-butoxycarbonylamino)piperidin-1-yl)quinolin-2-yl)imidazo[1,2-a]pyridine-7-carboxylate: Prepared according to Example 37, Step D using methyl 3-(8-bromoquinolin-2-yl)imidazo[1,2-a]pyridine-7-carboxylate in place of 8-bromo-2-(7-(pyridine-3-yl)imidazo[1,2-a]pyridine-3-yl)quinoline. MS APCI (+) m/z 502.1 (M+1) detected.

Step D: Preparation of methyl 3-(8-(4-aminopiperidin-1-yl)quinolin-2-yl)imidazo[1,2-a]pyridine-7-carboxylate: Prepared according to Example 37, Step E, using methyl 3-(8-(4-(tert-butoxycarbonylamino)piperidin-1-yl)quinolin-2-yl)imidazo[1,2-a]pyridine-7-carboxylate in place of tert-butyl 1-(2-(7-(pyridine-3-yl)imidazo[1,2-a]pyridine-3-yl)quinolin-8-yl)piperidin-4-ylcarbamate. MS APCI (+) m/z 402.2 (M+1) detected.

### Example 40

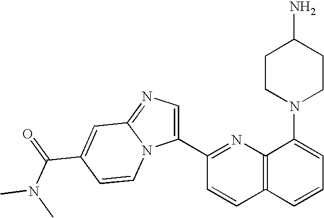

### 3-(8-(4-aminopiperidin-1-yl)quinolin-2-yl)-N,N-dimethylimidazo[1,2-a]pyridine-7-carboxamide

Step A: Preparation of 3-(8-bromoquinolin-2-yl)imidazo[1,2-a]pyridine-7-carboxylic acid hydrochloride salt: A solution of methyl 3-(8-bromoquinolin-2-yl)imidazo[1,2-a]pyridine-7-carboxylate (152 mg, 0.40 mmol) in 4.5 ml of a 8:1 THF:MeOH mixture was treated with aqueous lithium hydroxide (0.80 ml, 1.0 M, 0.80 mmol). The reaction mixture was stirred at ambient temperature for 21 hours, concentrated to dryness, then resuspended in excess 2.0 M HCl-ether. The volatiles were removed under reduced pressure, and the resulting material was dried under vacuum overnight to afford the title compound (197 mg, quantitative yield, accounting for the presence of 2 equivalents of LiCl) as a powder. MS ESI (+) m/z 368.3 and 370.3 (M+1 of each isotope) detected.

Step B: Preparation of 3-(8-bromoquinolin-2-yl)-N,N-dimethylimidazo[1,2-a]pyridine-7-carboxamide: A suspension of 3-(8-bromoquinolin-2-yl)imidazo[1,2-a]pyridine-7-carboxylic acid hydrochloride salt (containing 2 equivalents LiCl as an impurity, 100 mg, 0.247 mmol), and dimethylamine (18 mg, 0.40 mmol) in anhydrous DCM (3 ml) was treated sequentially with N-ethyl-N-isopropylpropan-2-amine (95.8 mg, 0.74 mmol) and HATU (100 mg, 0.26 mmol). The resulting mixture was stirred at ambient temperature overnight. Saturated aqueous sodium bicarbonate solution (3 mL) was added to the reaction and the resulting mixture was stirred at ambient temperature for 30 minutes. The reaction mixture was extracted with chloroform, dried over anhydrous sodium sulfate and concentrated to afford a solid. The solid was purified by silica gel chromatography (eluting with 8% MeOH-chloroform) to afford the desired compound (46 mg, 47% yield) as a solid. MS APCI (+) m/z 395.4 and 397.3 (M+1 for each isotope) detected.

Steps C-D: Preparation of 3-(8-(4-aminopiperidin-1-yl)quinolin-2-yl)-N,N-dimethylimidazo[1,2-a]pyridine-7-carboxamide: The desired compound was prepared from 3-(8-bromoquinolin-2-yl)-N,N-dimethylimidazo[1,2-a]pyridine-7-carboxamide following the procedures described for Steps D and E in Example 37. MS APCI (+) m/z 415.2 (M+1) detected.

### Example 41

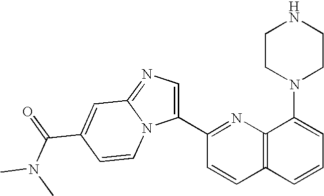

### N,N-dimethyl-3-(8-(piperazin-1-yl)quinolin-2-yl)imidazo[1,2-a]pyridine-7-carboxamide

Prepared according to the procedure for Example 40 using tert-butyl piperazine-1-carboxylate in place of tert-butyl piperidin-4-ylcarbamate. MS APCI (+) m/z 401.4 (M+1) detected.

### Example 42

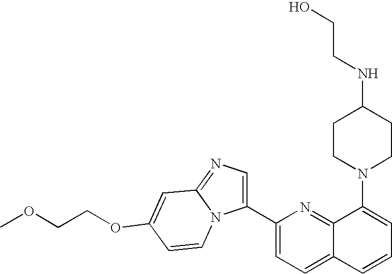

### 2-(1-(2-(7-(2-methoxyethoxy)imidazo[1,2-a]pyridin-3-yl)quinolin-8-yl)piperidin-4-ylamino)ethanol

Step A: Preparation of 1-(2-(7-(2-methoxyethoxy)imidazo[1,2-a]pyridin-3-yl)quinolin-8-yl)piperidin-4-one: 8-(2-(7-(2-Methoxyethoxy)imidazo[1,2-a]pyridin-3-yl)quinolin-8-yl)-1,4-dioxa-8-azaspiro[4.5]decane [prepared according to Example 1, step E, from 2-(7-(2-methoxyethoxy)imidazo[1,2-a]pyridin-3-yl)quinolin-8-yl trifluoromethanesulfonate and 1,4-dioxa-8-azaspiro[4.5]decane] was dissolved in a 1:1 THF-EtOH mixture and treated with concentrated aqueous HCl at ambient temperature. The reaction mixture was stirred at ambient temperature for nine days, then quenched with excess saturated aqueous sodium bicarbonate, extracted thoroughly with dichloromethane, chloroform, and ethyl acetate, dried over sodium sulfate, filtered and concentrated. The crude product was purified by silica gel chromatography (eluting with 3% MeOH-chloroform), and the major band was isolated to afford a solid (121 mg) containing 2-(7-(2-methoxyethoxy)imidazo[1,2-a]pyridin-3-yl)quinolin-8-ol and the title compound. MS APCI (+) m/z 417.3 (M+1) detected. This product mixture was used in subsequent reaction steps without further purification.

Step B: 2-(1-(2-(7-(2-methoxyethoxy)imidazo[1,2-a]pyridin-3-yl)quinolin-8-yl)piperidin-4-ylamino)ethanol: A mixture of 2-aminoethanol (6 mg, 0.1 mmol) and 1424742-methoxyethoxy)imidazo[1,2-a]pyridin-3-yl)quinolin-8-yl)piperidin-4-one (20 mg, 0.05 mmol) was dissolved in 0.5 ml of a 1:1 MeOH/THF mixture. The reaction mixture was stirred at ambient temperature under a nitrogen atmosphere overnight. Sodium tetrahydroborate (10 mg, 0.26 mmol) was added, and the reaction mixture stirred at ambient temperature for 5 hours. The reaction mixture was treated with 5 ml saturated aqueous sodium bicarbonate solution and extracted with 10 ml each of dichloromethane, chloroform, and ethyl acetate. The combined organic layers were dried over Na₂SO₄ and concentrated under reduced pressure to produce a solid. The solid was purified by silica gel chromatography (eluting with 10% MeOH-chloroform) to yield the title compound (0.4 mg, 1.8% yield). MS APCI (+) m/z 462.2 (M+1) detected.

### Example 43

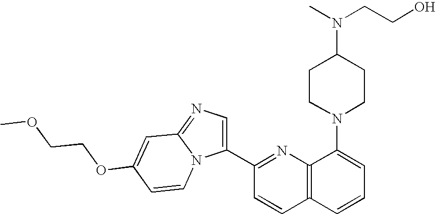

### 2-((1-(2-(7-(2-methoxyethoxy)imidazo[1,2-a]pyridin-3-yl)quinolin-8-yl)piperidin-4-yl)(methyl)amino)ethanol

Prepared according to Example 42 using 2-(methylamino)ethanol in place of 2-aminoethanol. MS APCI (+) m/z 476.2 (M+1) detected.

### Example 44

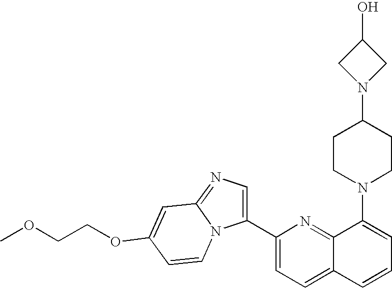

### 1-(1-(2-(7-(2-methoxyethoxy)imidazo[1,2-a]pyridin-3-yl)quinolin-8-yl)piperidin-4-yl)azetidin-3-ol

A mixture of azetidin-3-ol hydrochloride salt (10.5 mg, 0.10 mmol) and 1-(2-(7-(2-methoxyethoxy)imidazo[1,2-a]pyridin-3-yl)quinolin-8-yl)piperidin-4-one (Example 42; 20 mg, 0.05 mmol) was dissolved in 0.5 ml of a 1:1 MeOH:THF mixture. The reaction mixture was treated with N-ethyl-N-isopropylpropan-2-amine (13 mg, 0.10 mmol) and stirred at ambient temperature under a nitrogen atmosphere overnight. Sodium tetrahydroborate (10 mg, 0.26 mmol) was added, and the reaction mixture stirred at ambient temperature for 5 hours. The reaction mixture was treated with 5 ml saturated aqueous sodium bicarbonate solution and extracted with 10 ml each of dichloromethane, chloroform, and ethyl acetate. The combined organic layers were dried over Na₂SO₄ and concentrated under reduced pressure to produce a solid. The solid was purified by silica gel chromatography (eluting with 10% MeOH-chloroform) to yield the title compound (5.0 mg, 15% yield). MS APCI (+) m/z 474.3 (M+1) detected.

### Example 45

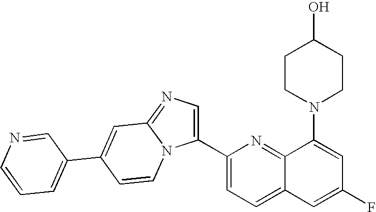

### 1-(6-fluoro-2-(7-(pyridin-3-yl)imidazo[1,2-a]pyridin-3-yl)quinolin-8-yl)piperidin-4-ol

Step A: Preparation of 3,4′-bipyridin-2′-amine: A reaction container with a screw cap was charged with 4-bromopyridin-2-amine (2.51 g, 14.5 mmol), pyridin-3-ylboronic acid (2.67 g, 21.8 mmol), sodium 2′-(dicyclohexylphosphino)-2,6-dimethoxybiphenyl-3-sulfonate (0.149 g, 0.290 mmol), diacetoxypalladium (0.0326 g, 0.145 mmol) and K₂CO₃ (6.02 g, 43.5 mmol). A septum was attached and the container was evacuated and back filled with Ar three times. In a separate flask was charged with 30 mL H₂O. The flask was degassed under vacuum for 10 minutes. The degassed H₂O was then added to the reaction container, which was then flashed with Ar and capped. Heat to 100° C. and stir for 12 hours. Cool to ambient temperature and extracted with EtOAc (3×60 mL). Combine and flash chromatography (EtOAc/MeOH 20:1) provided final product.

Step B: Preparation of 1-(6-fluoro-2-(7-(pyridin-3-yl)imidazo[1,2-a]pyridin-3-yl)quinolin-8-yl)piperidin-4-ol: Prepared as described in Example 26 substituting 3,4′-bipyridin-2′-amine for 4-(2-methoxyethoxy)pyridin-2-amine and piperidin-4-ol for tert-butyl piperidin-4-ylcarbamate. MS APCI (+) m/z 440.3 (M+1) detected.

### Example 46

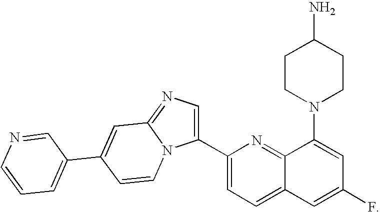

### 1-(6-fluoro-2-(7-(pyridin-3-yl)imidazo[1,2-a]pyridin-3-yl)quinolin-8-yl)piperidin-4-amine

Prepared as described in Example 26 substituting 3,4′-bipyridin-2′-amine for 4-(2-methoxyethoxy)pyridin-2-amine. MS APCI (+) m/z 439.2 (M+1) detected.

### Example 47

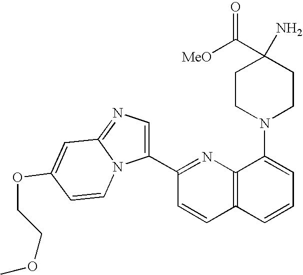

### Methyl 4-amino-1-(2-(7-(2-methoxyethoxy)imidazo pyridin-3-yl)quinolin-8-yl)piperidine-4-carboxylate

Step A: Preparation of methyl 4-(benzyloxycarbonylamino)piperidine-4-carboxylate: Prepared from 1-tert-butyl 4-methyl 4-(benzyloxycarbonylamino)piperidine-1,4-dicarboxylate according the procedure of Example 1, step F.

Step B: Preparation of methyl 4-(benzyloxycarbonylamino)-1-(2-(7-(2-methoxyethoxy)imidazo[1,2-a]pyridin-3-yl)quinolin-8-yl)piperidine-4-carboxylate: Prepared according to Example 27, using methyl 4-(benzyloxycarbonylamino)piperidine-4-carboxylate in place of benzyl (cis)-3-fluoropiperidin-4-ylcarbamate. MS APCI (+) m/z 610.3 (M+1) detected.

Step C: Preparation of methyl 4-amino-1-(2-(7-(2-methoxyethoxy)imidazo[1,2-a]pyridin-3-yl)quinolin-8-yl)piperidine-4-carboxylate: The Cbz group was removed according to the procedure of Example 27, step E. MS APCI (+) m/z 476.2 (M+1) detected.

### Example 48

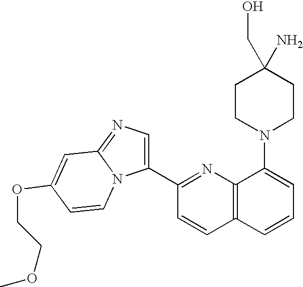

### (4-Amino-1-(2-(7-(2-methoxyethoxy)imidazo[1,2-a]pyridin-3-yl)quinolin-8-yl)piperidin-4-yl)methanol

LiAlH₄ (0.78 ml, 0.78 mmol) was added to a solution of methyl 4-amino-1-(2-(7-(2-methoxyethoxy)imidazo[1,2-a]pyridin-3-yl)quinolin-8-yl)piperidine-4-carboxylate (Example 47; 0.185 g, 0.389 mmol) in THF. The reaction was cooled to 0° C., the ice bath was removed and the reaction was stirred for 1 hour. The reaction mixture was treated with 1:1 sodium sulfate decahydrate/celite mixture. Chloroform was added, and the mixture was filtered, condensed and purified by silica gel chromatography to provide 66 mg of the desired product. MS APCI (+) m/z 448.2 (M+1) detected.

### Example 49

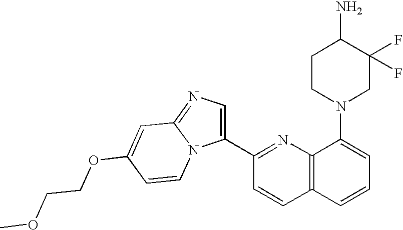

### 3,3-Difluoro-1-(2-(7-(2-methoxyethoxy)imidazo[1,2-a]pyridin-3-yl)quinolin-8-yl)piperidin-4-amine

### 1. Preparation of 2-(7-(2-methoxyethoxy)imidazo[1,2-a]pyridin-3-yl)quinolin-8-yl trifluoromethanesulfonate

Step 1A: Preparation of 8-(benzyloxy)quinolin-2-ol: To a 500 ml flask was added quinoline-2,8-diol (20.0 g, 124.1 mmol), K₂CO₃ (17.15 g, 124.1 mmol), benzyl bromide (14.76 ml, 124.1 mmol) and DMF (124.1 ml, 124.1 mmol). The mixture was heated to 65° C. overnight, then poured into 1000 ml water and stirred for 5 hours. The solids were collected by filtration and washed with 1000 ml diethyl ether to yield 26.5 g (85% yield) of desired product.

Step 1B: Preparation 8-(benzyloxy)-2-chloroquinoline: A 500 mL flask was charged with 8-(benzyloxy)quinolin-2-ol (26.5 g, 105 mmol) and DCE (105 ml, 105 mmol). Oxalyl chloride (18.4 ml, 211 mmol) was added dropwise, then add a couple of drops of DMF (0.5 ml, 105 mmol) were added. The reaction was heated to 85° C. overnight. The reaction was cooled to ambient temperature and concentrated to an oil. DCM (300 mL) was added to the oil and the organic layer was washed with 300 ml of saturated NaHCO₃. The layers were separated, the organic phase was dried over Na₂SO₄, filtered and concentrated to an oil. The residue was crystallized from toluene to yield 28.4 g of desired product (quantitative yield).

Step 1C: Preparation of 8-(benzyloxy)-2-(7-(2-methoxyethoxy)-imidazo[1,2-a]pyridin-3-yl)quinoline: 8-(Benzyloxy)-2-chloroquinoline (5.0 g, 18.5 mmol), 7-(2-methoxyethoxy)-imidazo[1,2-a]pyridine (3.56 g, 18.5 mmol), Pd(PPh₃)₄ (1.07 g, 0.927 mmol), K₂CO₃ (5.12 g, 37.1 mmol), and Pd(OAc)₂ (0.208 g, 0.927 mmol) were added to dioxane (74.1 ml, 18.5 mmol) and water (0.735 ml, 40.8 mmol) and heated to 100° C. overnight under nitrogen. The reaction was then diluted with DCM and carbon (5 g) was added. The reaction mixture was filtered and the filtrate was triturated with 1:1 EtOAc/MTBE (30 mL). The resulting solids were allowed to stir for 5 hours and were then filtered to isolate the desired product as a solid (5.4 g, 69% yield).

Step 1D: Preparation of 2-(7-(2-methoxyethoxy)imidazo[1,2-a]pyridin-3-yl)quinolin-8-ol: 8-(Benzyloxy)-2-(7-(2-methoxyethoxy)-imidazo[1,2-a]pyridin-3-yl)quinoline (5.0 g, 11.75 mmol) was slurried in MeOH (117.5 ml). Ammonium formate (7.410 g, 117.5 mmol) and Pd(OH)₂/C (0.8252 g, 0.5876 mmol) were added. The reaction was heated to reflux for 2 hours until reaction was complete by TLC (100% ethyl acetate). Reaction mixture was cooled to 20° C. and formic acid was added to the slurry until the solids went into solution. The solution was filtered and washed with 100 ml 10% formic acid in methanol. The filtrate was concentrated to an oil. To the oil was added an excess of NH₃ in methanol and the resulting solids were concentrated to dryness. Water was added and solids were allowed to stir for 1 hour (pH was 6.5-7.0). The solution was filtered and the solids were taken up in toluene and concentrated to dryness. The solids were dried under vacuum dry for 12 hours to obtain 3.8 g. (96% yield).

Step 1E: Preparation of 2-(7-(2-methoxyethoxy)imidazo[1,2-a]pyridin-3-yl)quinolin-8-yl trifluoromethanesulfonate: To a solution of 2-(7-(2-methoxyethoxy)imidazo[1,2-a]pyridin-3-yl)quinolin-8-ol (40 g, 119 mmol), triethylamine (33.3 ml, 238 mmol) and DMF (300 ml) was added 1,1,1-trifluoro-N-phenyl-N-(trifluoromethylsulfonyl)methanesulfonamide (136.4 g, 381.6 mmol). The resulting solution was allowed to stir for 24 hours. The solids were filtered and washed with ether to yield desired product (41.2 g, 74% yield). ¹H NMR (DMSO-d₆) 10.16 (1H, d J 6.8 Hz), 8.65 (1H, s), 8.51 (1H, d, J=8.6 Hz), 8.30 (1H, d, J=8.6 Hz), 8.08 (1H, d, J 8.0), 7.87 (1H, d, J 7.6), 7.64 (1H, m), 7.24 (1H, s), 6.81 (1H, m), 4.29 (2H, M), 3.73 (2H, M), 3.34 (3H, s).

### 2. Preparation of 3,3-difluoro-1-(2-(7-(2-methoxyethoxy)imidazo[1,2-a]pyridin-3-yl)quinolin-8-yl)piperidin-4-amine

Step 2A: Preparation of 1-benzyl-3,3-difluoropiperidine-4,4-diol: Ethyl 1-benzyl-5,5-difluoro-4-oxopiperidine-3-carboxylate (2.00 g, 6.73 mmol) [Bezencon, O.; et al.; WO 2005/040120] was dissolved in 3 N HCl (20 mL) and heated to reflux for 20 hours. The reaction was cooled, solid NaHCO₃ was added to adjust to pH 8, and the solution was extracted with Et₂O. The combined organic phase was washed with saturated NaCl, dried over Na₂SO₄, filtered and concentrated in vacuo to a solid (1.54 g). MS APCI (+) m/z 244.0 (M+1) detected.

Step 2B: Preparation of tert-butyl 4-(benzylamino)-3,3-difluoropiperidine-1-carboxylate: 1-Benzyl-3,3-difluoropiperidine-4,4-diol (0.34 g, 1.42 mmol) was dissolved in 95% EtOH (7 mL) and treated with di-tert-butyl dicarbonate (0.62 g, 2.8 mmol) and 10% palladium on carbon (Degeussa type, 35 mg). The reaction was placed under a balloon of hydrogen and stirred for 2 hours. The reaction mixture was filtered through a nylon membrane (45 μM), washed with ethanol, and concentrated in vacuo to an oil. tert-Butyl 3,3-difluoro-4,4-dihydroxypiperidine-1-carboxylate, was carried forward without purification. tert-Butyl 3,3-difluoro-4,4-dihydroxypiperidine-1-carboxylate (0.100 g, 0.394 mmol) was dissolved in methylene chloride (1.2 mL) and treated with benzylamine (0.063 g, 0.59 mmol) and NaBH(OAc)₃ (0.167 g, 0.789 mmol). The mixture was stirred at ambient temperature for 16 hours. The reaction was acidified with 3 N HCl and stirred for 20 minutes, neutralized to pH 8 with solid NaHCO₃, and then separated. The aqueous layer was washed with methylene chloride and the combined organic layers were washed with 6% NaHCO₃ solution, dried over Na₂SO₄, filtered and concentrated in vacuo to provide the desired product (210 mg).

Step 2C: Preparation of N-benzyl-3,3-difluoropiperidin-4-amine: tert-Butyl 4-(benzylamino)-3,3-difluoropiperidine-1-carboxylate (0.18 g, 0.56 mmol) was dissolved in MeOH (1 mL) and cooled to 0° C. then treated with 4 M HCl in dioxane (2.11 ml, 8.46 mmol). The reaction mixture was stirred at 0° C. for a few minutes then warmed to ambient temperature and stirred for 4 hours. The mixture was concentrated in vacuo from MeOH three times and the solids were re-dissolved in a mixture of methylene chloride (2 mL) and 1 N NaOH (2 mL), then stirred for 20 minutes. The organic layer was separated and the aqueous was washed twice with methylene chloride. The combined organic phase was dried over Na₂SO₄, filtered and concentrated in vacuo to provide the desired product (71.7 mg). MS APCI (+) m/z 227.1 (M+1) detected.

Step 2D: Preparation of N-benzyl-3,3-difluoro-1-(2-(7-(2-methoxyethoxy)imidazo[1,2-a]pyridin-3-yl)quinolin-8-yl)piperidin-4-amine: N-Benzyl-3,3-difluoropiperidin-4-amine (0.071 g, 0.31 mmol) was combined with 2-(7-(2-methoxyethoxy)imidazo[1,2-a]pyridin-3-yl)quinolin-8-yl trifluoromethanesulfonate (Steps 1A -1E; 0.113 g, 0.243 mmol), micronized Cs₂CO₃ (0.111 g, 0.341 mmol), BINAP-racemic (0.0151 g, 0.0243 mmol) and Pd₂ dba₃ (0.011 g, 0.012 mmol). The mixture was treated with toluene (1.5 mL), degassed with argon and heated to reflux for 16 hours. The reaction was cooled, diluted with CHCl₃ and applied directly to silica gel column. The column was eluted with a gradient from 1-20% (6% NH₄OH in MeOH)/ethyl acetate, (130 mg). MS APCI (+) m/z 544.2 (M+1) detected.

Step 2E: Preparation of 3,3-difluoro-1-(2-(7-(2-methoxyethoxy)imidazo[1,2-a]pyridin-3-yl)quinolin-8-yl)piperidin-4-amine: N-Benzyl-3,3-difluoro-1-(2-(7-(2-methoxyethoxy)imidazo[1,2-a]pyridin-3-yl)quinolin-8-yl)piperidin-4-amine (0.035 g, 0.064 mmol) was treated with 20% Pd(OH)₂ on carbon (0.009 g, 0.064 mmol) and ammonium formate (0.406 g, 6.43 mmol) then slurried in 95% EtOH (2.1 mL). The reaction mixture was sealed and heated to 80° C. for 16 hours. The reaction was cooled then diluted with CHCl₃ and water. The solution was filtered through a nylon membrane (0.45 μM). The layers were separated then the organic layer was washed with water then dried over Na₂SO₄, filtered and concentrated in vacuo to a solid. This material was purified by silica gel chromatography, eluting with a mixture of 6% NH₄OH in MeOH/ethyl acetate to provide the desired product as a solid (5.9 mg). ¹H NMR (400 MHz, CDCl₃) δ 10.43 (d, 1H), 8.23 (s, 1H), 8.14 (d, 1H), 7.82 (d, 1H), 7.51 (d, 1H), 7.44 (t, 1H), 7.19-7.12 (m, 1H), 6.95-6.90 (m, 1H), 4.31-4.22 (m, 2H), 3.88-3.80 (m, 2H), 3.49 (s, 3H), 3.22-3.08 (m, 1H), 3.08-2.95 (m, 1H), 2.22-1.92 (m, 2H). MS APCI (+) m/z 454.3 (M+1) detected.

### Example 50

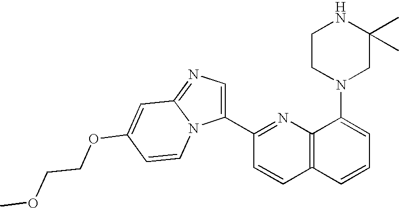

### 8-(3,3-Dimethylpiperazin-1-yl)-2-(7-(2-methoxyethoxy)imidazo[1,2-a]pyridin-3-yl)quinoline bis-hydrochloride

Step A: Preparation of tert-butyl 4-(2-(7-(2-methoxyethoxy)imidazo[1,2-a]pyridin-3-yl)quinolin-8-yl)-2,2-dimethylpiperazine-1-carboxylate: Prepared according to the method of Example 27, using tert-butyl 2,2-dimethylpiperazine-1-carboxylate in place of benzyl cis-4-amino-3-fluoropiperidine-1-carboxylate. MS APCI (+) m/z 532.1 (M+1) detected.

Step B: Preparation of 8-(3,3-dimethylpiperazin-1-yl)-2-(7-(2-methoxyethoxy)imidazo[1,2-a]pyridin-3-yl)quinoline: tert-Butyl 4-(2-(7-(2-methoxyethoxy)imidazo[1,2-a]pyridin-3-yl)quinolin-8-yl)-2,2-dimethylpiperazine-1-carboxylate (0.037 g, 0.070 mmol) was dissolved in MeOH (0.5 mL), cooled to 0° C., and treated with 4 M HCl in dioxane (0.44 ml, 1.7 mmol). The solution was stirred at ambient temperature for 3 hours. The reaction was cooled to 0° C. and concentrated in vacuo. The residue was suspended in MeOH and concentrated three times. The residue was purified by silica gel chromatography, eluting with a gradient from 1-20% (6% NH₄OH in MeOH)/ethyl acetate. The desired product was dissolved in MeOH and treated with 4 M HCl in dioxane (0.5 mL). The mixture was concentrated in vacuo, dissolved and concentrated from MeOH three times. The salt was dissolved in MeOH (0.2 mL) then added dropwise into Et₂O (20 mL). The resultant solid was filtered, washed with Et₂O and dried under nitrogen gas to a solid (11.4 g). NMR (400 MHz, CD₃OD) δ 10.47 (d, J=7.7 Hz, 1H), 8.69 (s, 1H), 8.47 (d, J=8.7 Hz, 1H), 8.07 (d, J=9.0 Hz, 1H), 7.72 (d, 1H), 7.62 (t, J=7.4 Hz, 1H), 7.48-7.41 (m, 2H), 7.32-7.28 (m, 1H), 4.47-4.43 (m, 2H), 3.88-3.85 (m, 2H), 3.61-3.53 (m, 1H), 3.53-3.47 (m, 2H), 3.45 (s, 3H), 3.39-3.36 (m, 2H), 1.65 (s, 6H). MS APCI (+) m/z 432.2 (M+1) detected

### Example 51

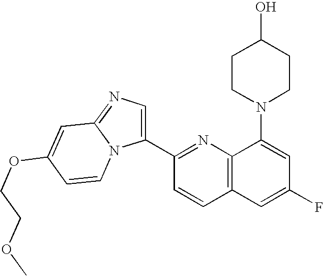

### 1-(6-Fluoro-2-(7-(2-methoxyethoxy)imidazo[1,2-a]pyridin-3-yl)quinolin-8-yl)piperidin-4-ol

Prepared according to the procedure of Example 26 using piperidin-4-ol in place of tert-butyl piperidin-4-ylcarbamate. APCI (+) m/z 437.3 (M+1) detected.

### Example 52

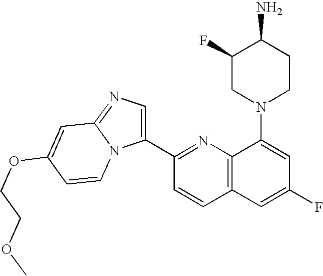

### (cis)-3-fluoro-1-(6-fluoro-2-(7-(2-methoxyethoxy)imidazo[1,2-a]pyridin-3-yl)quinolin-8-yl)piperidin-4-amine

Prepared according to Example 27 using 8-bromo-6-fluoro-2-(7-(2-methoxyethoxy)H-imidazo[1,2-a]pyridin-3-yl)quinoline in place of 2-(7-(2-methoxyethoxy)imidazo[1,2-a]pyridin-3-yl)quinolin-8-yl trifluoromethanesulfonate. APCI (+) m/z 454.2 (M+1) detected.

### Example 53

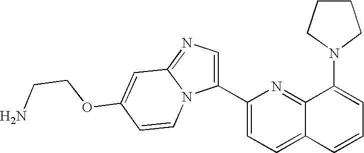

### 2-(3-(8-(pyrrolidin-1-yl)quinolin-2-yl)imidazo[1,2-a]pyridin-7-yloxy)ethanamine

Step A: Preparation of 2-(2-(3-(8-(pyrrolidin-1-yl)quinolin-2-yl)imidazo[1,2-a]pyridin-7-yloxy)ethyl)isoindoline-1,3-dione: Prepared according to the procedure for Example 1, using 2-(2-hydroxyethyl)isoindoline-1,3-dione in place of 2-methoxyethanol and pyrrolidine in place of tert-butyl piperazine-1-carboxylate.

Step B: Preparation of 2-(3-(8-(pyrrolidin-1-yl)quinolin-2-yl)imidazo[1,2-a]pyridin-7-yloxy)ethanamine: To 2-(2-(3-(8-(pyrrolidin-1-yl)quinolin-2-yl)imidazo[1,2-a]pyridin-7-yloxy)ethyl)isoindoline-1,3-dione (60 mg, 0.12 mmol) in EtOH (3 mL) was added methylhydrazine (27 mg, 0.60 mmol). The reaction was heated to reflux for 3 hours, then cooled and concentrated. The residue was purified by silica gel column chromatography (DCM/MeOH/NH₄OH 10:1:0.1) to provide the desired product (18 mg). APCI (+) m/z 374.1 (M+1) detected.

### Example 54

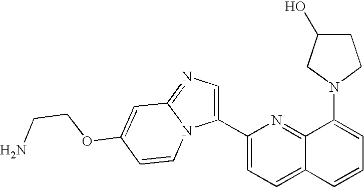

### 1-(2-(7-(2-aminoethoxy)imidazo[1,2-a]pyridin-3-yl)quinolin-8-yl)pyrrolidin-3-ol

Prepared according to the procedure for Example 53 using pyrrolidin-3-ol in place of pyrrolidine. APCI (+) m/z 390.1 (M+1) detected.

### Example 55

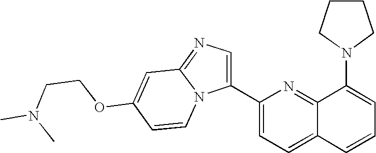

### N,N-dimethyl-2-(3-(8-(pyrrolidin-1-yl)quinolin-2-yl)imidazo[1,2-a]pyridin-7-yloxy)ethanamine

To 2-(3-(8-(pyrrolidin-1-yl)quinolin-2-yl)imidazo[1,2-a]pyridin-7-yloxy)ethanamine (Example 53; 10 mg, 0.027 mmol) in MeOH/DCM (1 ml/1 ml) was added HCHO (8.0 mg, 0.27 mmol) and Na(OAc)₃BH (17 mg, 0.080 mmol). The reaction was stirred for 1 hour, then concentrated and diluted with saturated NaHCO₃ (5 ml) and DCM (10 ml). The aqueous layer was extracted with DCM, and the combined organic layers were dried, filtered and concentrates. The residue was purified by silica gel chromatography (DCM/MeOH/NH₄OH 10:1:0.1) to provided final product (6 mg, 56%). APCI (+) m/z 402.1 (M+1) detected.

### Example 56

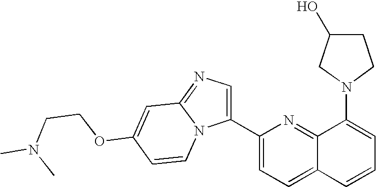

### 1-(2-(7-(2-(Dimethylamino)ethoxy)imidazo[1,2-a]pyridin-3-yl)quinolin-8-yl)pyrrolidin-3-ol

Prepared according to the procedure for Example 55. APCI (+) m/z 418.1 (M+1) detected.

### Example 57

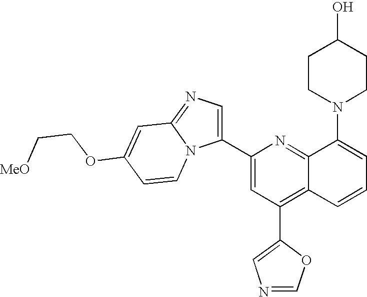

### 1-(2-(7-(2-methoxyethoxy)imidazo[1,2-a]pyridin-3-yl)-4-(oxazol-5-yl)quinolin-8-yl)piperidin-4-ol

### 1. Preparation of tert-butyl 2-ethynyl-6-methoxyphenylcarbamate

Step 1A: Preparation of tert-butyl 2-iodo-6-methoxyphenylcarbamate: To tert-butyl 2-methoxyphenylcarbamate (24.1 g, 108 mmol) in dry Et₂O (100 mL) at −20° C. was added dropwise tert-butyllithium (140 ml, 237 mmol). The clear solution turned cloudy at the end of the addition. The reaction was stirred for 3 hours at −20° C., then cooled to −100° C. with a liquid N₂/Et₂O bath. Iodine (27.4 g, 108 mmol) in Et₂O (250 mL) was added to the solution. Following addition of I₂, the reaction was slowly warmed to ambient temperature over night. Na₂S₂O₃ (saturated, 200 mL) was then added to the reaction mix and phases were separated. The aqueous was extracted with Et₂O, and the combined organic layers were dried (MgSO₄), filtered and concentrated. DCM (50 mL) was added, followed by hexanes (200 mL). The solution was concentrated to remove DCM. The product crashed out, and was collected by filtration and washed with hexanes (100 mL) to give the crude product (58%).

Step 1B: Preparation of tert-butyl 2-methoxy-6-((trimethylsilyl)ethynyl)phenylcarbamate: To tert-butyl 2-iodo-6-methoxyphenylcarbamate (10.36 g, 29.671 mmol), ethynyltrimethylsilane (3.2056 g, 32.638 mmol), copper(I) iodide (0.282 g, 1.483 mmol), and PdCl₂(PPh₃)₂ (1.0413 g, 1.4835 mmol) in THF (100 mL) was added triethylamine (3.6029 g, 35.605 mmol), followed by overnight stirring. The crude reaction was then concentrated and the mixture was flashed through silica gel with 10:1 Hex/EtOAc to give the desired product (98%).

Step 1C: Preparation of tert-butyl 2-ethynyl-6-methoxyphenylcarbamate: To tert-butyl 2-methoxy-6-((trimethylsilyl)ethynyl)phenylcarbamate (4.21 g, 13.2 mmol) in MeOH (30 mL) was added K₂CO₃ (9.11 g, 65.9 mmol). The reaction was stirred for 30 minutes, then filtered and washed with DCM (50 mL). The combined organic layers were concentrated and diluted with DCM (20 mL), filtered, washed a second time with DCM (50 mL), then concentrated. The residue was purified by flash chromatography through a pad of silica gel with 10:1 Hexane/EtOAc (500 mL), affording the desired product (62%).

### 2. Preparation of N-methoxy-7-(2-methoxyethoxy)-N-methylimidazo[1,2-a]pyridine-3-carboxamide

Step 2A: Preparation of ethyl 7-(2-methoxyethoxy)imidazo[1,2-a]pyridine-3-carboxylate: Ethyl 2-chloro-3-oxopropanoate (5.1 g, 33.9 mmol, Heterocycles 1991, pg. 699) and 4-(2-methoxyethoxy)pyridin-2-amine (5.70 g, 33.9 mmol) was dissolved in EtOH (50 mL) and heated to reflux overnight. The crude reaction mixture was concentrated and purified by flash column chromatography (EtOAc/MeOH 10:0 to 10:1) provided the desired product (57%).

Step 2B: Preparation of 7-(2-methoxyethoxy)imidazo[1,2-a]pyridine-3-carboxylic acid: To ethyl 7-(2-methoxyethoxy)imidazo[1,2-a]pyridine-3-carboxylate (5.01 g, 19.0 mmol) in THF/EtOH (32/6 mL) was added lithium hydroxide (37.9 ml, 37.9 mmol), and the reaction was stirred overnight. HCl (57 mmol, 2 M in ether) was added to the mixture, followed by concentration to give the desired product.

Step 2C: Preparation of N-methoxy-7-(2-methoxyethoxy)-N-methylimidazo[1,2-a]pyridine-3-carboxamide: To EDCI (2.1960 g, 11.455 mmol) and HOBT-H₂O (1.754 g, 11.455 mmol) in DMF (50 mL) was added N-ethyl-N-isopropylpropan-2-amine (1.480 g, 11.455 mmol), followed by the addition of N,O-dimethylhydroxylamine hydrochloride (1.117 g, 11.455 mmol). The reaction was stirred overnight, followed by concentration to remove most of the DMF. The crude mixture was diluted with saturated NaHCO₃ (20 mL)/EtOAc (40 mL). The aqueous phase was ten extracted with EtOAc, dried over Na₂SO₄ and concentrate to give the desired product (72%).

### 3. Preparation of 1-(2-(7-(2-methoxyethoxy)imidazo[1,2-a]pyridin-3-yl)-4-(oxazol-5-yl)quinolin-8-yl)piperidin-4-ol

Step 3A: Preparation of tert-butyl 2-methoxy-6-(3-(7-(2-methoxyethoxy)imidazo[1,2-a]pyridin-3-yl)-3-oxoprop-1-ynyl)phenylcarbamate: To tert-butyl 2-ethynyl-6-methoxyphenylcarbamate (1.77 g, 7.18 mmol) in THF (40 mL) was added butyllithium (0.919 g, 14.4 mmol) at −78° C., and the reaction was stirred for 1 hour. N-methoxy-7-(2-methoxyethoxy)-N-methylimidazo[1,2-a]pyridine-3-carboxamide (1.67 g, 5.98 mmol) in THF (55 mL) was then added to the reaction mixture dropwise. After the addition, the cold bath was removed and the reaction was warmed to ambient temperature. Following a 2 hour stir at ambient temperature, the reaction mixture was poured into cold saturated NH₄Cl (40 mL) and EtOAc (50 mL). The phases were separated and the aqueous phase was extracted with EtOAc, dried over Na₂SO₄, filtered and concentrated. The residue was triturated with DCM to give product as a solid. The DCM solution was concentrated and purified by flash column chromatography (EtOAc/MeOH 10:0 to 10:1) to provide the desired product.

Step 3B: Preparation of 4-iodo-8-methoxy-2-(7-(2-methoxyethoxy)imidazo[1,2-a]pyridin-3-yl)quinoline: To tert-butyl 2-methoxy-6-(3-(7-(2-methoxyethoxy)imidazo[1,2-a]pyridin-3-yl)-3-oxoprop-1-ynyl)phenylcarbamate (2.51 g, 5.39 mmol) and sodium iodide (16.2 g, 108 mmol) was added acetic acid/formic acid (5 mL/5 mL). The reaction vessel was purged with N₂ and heated to 60° C. for 3 hours. The reaction was then cooled to ambient temperature and diluted with H₂O/DCM (50 mL/100 mL), followed by extraction with DCM. The combined organics were washed with saturated NaHCO₃, dried over Na₂SO₄, filtered and concentrated in vacuo. The residue was purified by flash column chromatography (EtOAc/MeOH 10:1) provided the desired product (92%).

Step 3C: Preparation of 8-methoxy-2-(7-(2-methoxyethoxy)imidazo[1,2-a]pyridin-3-yl)-4-vinylquinoline: To 4-iodo-8-methoxy-2-(7-(2-methoxyethoxy)imidazo[1,2-a]pyridin-3-yl)quinoline (898 mg, 1.89 mmol) in NMP (10 mL) was added Pd₂ dba₃ (86.508 mg, 0.094471 mmol), trifuran-2-ylphosphine (87.734 mg, 0.37788 mmol) and tributyl(vinyl)stannane (659.04 mg, 2.0784 mmol). The reaction flask was purged with N₂ and the reaction was stirred at 80° C. for 2 hours. The crude mixture was diluted with EtOAc (30 mL) then washed with H₂O, dried over Na₂SO₄ and concentrated. The residue was purified by flash column chromatography (EtOAc/Hexane 8:1) affording the desired product (80%).

Step 3D: Preparation of 1-(8-methoxy-2-(7-(2-methoxyethoxy)imidazo[1,2-a]pyridin-3-yl)quinolin-4-yl)ethane-1,2-diol: To 8-methoxy-2-(7-(2-methoxyethoxy)imidazo[1,2-a]pyridin-3-yl)-4-vinylquinoline (656 mg, 1.75 mmol) in DCM (20 mL) at 0° C. was added dropwise a solution of triethylbenzylammonium chloride (504 mg, 2.62 mmol) and KMnO₄ (414 mg, 2.62 mmol) in DCM (40 mL), and the reaction was stirred for 2 hours at 0° C. The reaction mixture was then warmed to ambient temperature and treated with 3% NaOH (30 mL). The mixture was filtered through celite and washed with DCM (100 mL), followed by extraction with DCM. The combined organic phases were dried over Na₂SO₄, filtered and concentrated to give the desired product (44%).

Step 3E: Preparation of 8-methoxy-2-(7-(2-methoxyethoxy)imidazo[1,2-a]pyridin-3-yl)quinoline-4-carbaldehyde: To silica gel (1.5 g) in DCM (5 mL) was added dropwise sodium periodate (131 μl, 0.850 mmol), affording a slurry after the addition. 1-(8-Methoxy-2-(7-(2-methoxyethoxy)imidazo[1,2-a]pyridin-3-yl)quinolin-4-yl)ethane-1,2-diol (232 mg, 0.567 mmol) in DCM (3 mL) was added to the slurry, followed by 30 minute stir. The mixture was then filtered, washed with DCM (10 mL), and concentrated to give the desired product (100%).

Step 3F: Preparation of 5-(8-methoxy-2-(7-(2-methoxyethoxy)imidazo[1,2-a]pyridin-3-yl)quinolin-4-yl)oxazole: To 8-Methoxy-2-(7-(2-methoxyethoxy)imidazo[1,2-a]pyridin-3-yl)quinoline-4-carbaldehyde (210 mg, 0.556 mmol) and 1-(isocyanomethylsulfonyl)-4-methylbenzene (130 mg, 0.668 mmol) in MeOH (5 mL) was added K₂CO₃ (154 mg, 1.11 mmol), followed by heating to reflux for 3 hours. The reaction was then cooled to ambient temperature, concentrated and purified by flash column chromatography (EtOAc/MeOH 10:1) providing the desired product (73%). MS APCI (+) m/z 417.2 (M+1) detected.

Step 3G: Preparation of 2-(7-(2-methoxyethoxy)imidazo[1,2-a]pyridin-3-yl)-4-(oxazol-5-yl)quinolin-8-ol: To 5-(8-methoxy-2-(7-(2-methoxyethoxy)imidazo[1,2-a]pyridin-3-yl)quinolin-4-yl)oxazole (80 mg, 0.19 mmol) in DMF (3 mL) was added sodium ethanethiolate (162 mg, 1.9 mmol). The reaction vial was sealed and heated to 150° C. for 2 hours. The reaction was then cooled to ambient temperature and concentrated. The residue was purified by flash column chromatography (DCM/MeOH 10:1) providing the desired product (39%).

Step 3H: Preparation of 2-(7-(2-methoxyethoxy)imidazo[1,2-a]pyridin-3-yl)-4-(oxazol-5-yl)quinolin-8-yl trifluoromethanesulfonate: To 2-(7-(2-methoxyethoxy)imidazo[1,2-a]pyridin-3-yl)-4-(oxazol-5-yl)quinolin-8-ol (20 mg, 0.050 mmol) in DMF (2 mL) was added triethylamine (10 mg, 0.099 mmol) and 1,1,1-trifluoro-N-phenyl-N-(trifluoromethylsulfonyl)methanesulfonamide (27 mg, 0.075 mmol). The reaction was stirred for 24 hours, then concentrated. The residue was purified by silica gel column chromatography (DCM/MeOH 10:1) provided the desired product (17 mg).

Step 3I: Preparation of 1-(2-(7-(2-methoxyethoxy)imidazo[1,2-a]pyridin-3-yl)-4-(oxazol-5-yl)quinolin-8-yl)piperidin-4-ol: To a suspension of Pd₂ dba₃ (2.9 mg, 0.0032 mmol) in toluene (2 mL) was added binap-rac (5.9 mg, 0.0095 mmol). Argon was bubbled through the solution for 1 minute. The reaction was stirred under argon for 30 minutes. 2-(7-(2-Methoxyethoxy)imidazo[1,2-a]pyridin-3-yl)-4-(oxazol-5-yl)quinolin-8-yl trifluoromethanesulfonate (17 mg, 0.032 mmol), Cs₂CO₃ (31 mg, 0.095 mmol), and piperidin-4-ol (9.7 mg, 0.095 mmol) were added to the reaction mixture. The reaction was purged with argon for 2 minutes and heat at 100° C. for 8 hours. The reaction was cooled to ambient temperature and concentrated. The residue was purified by silica gel column chromatography (DCM/MeOH 10:1) to provide the desired product (4 mg). APCI (+) m/z 486.3 (M+1) detected.

### Example 58

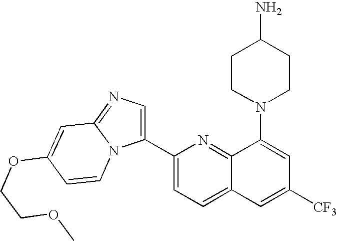

### 1-(2-(7-(2-methoxyethoxy)imidazo[1,2-a]pyridin-3-yl)-6-(trifluoromethyl)quinolin-8-yl)piperidin-4-amine

Step A: Preparation of 8-bromo-2-methyl-6-(trifluoromethyl)quinoline: 2-bromo-4-(trifluoromethyl)aniline (6.0 g, 25.0 mmol) was weighed into a 500 mL one neck round bottom flask, and dissolved in 50 mL of 6 N HCl. The reaction mixture was then heated to reflux, followed by drop-wise addition of (E)-but-2-enal (2.2 ml, 26.3 mmol) mixed with 1.0 mL de-ionized water over 25 minutes. Following complete addition the reaction was heated at 100° C. for an additional 35 minutes. The reaction was cooled to ambient temperature, followed by addition of 50 mL of Et₂O. The reaction was stirred for 5 minutes followed by removal of Et₂O by separatory funnel. The aqueous layer was replace into the original reaction flask and ZnCl₂ (3.407 g, 25.00 mmol) was then added in two portions followed by cooling to 0° C. over 30 minutes. The aqueous layer was then cooled to 0° C., and taken to pH=8.0 using concentrated NH₄OH. The aqueous was then extracted with Et₂O and then EtOAc. The combined organic phases were dried over Na₂SO₄ and concentrated in vacuo, affording the desired product (2.0 g, 6.9 mmol, 28% yield) as a solid.

Step B: Preparation of 8-bromo-6-(trifluoromethyl)quinoline-2-carbaldehyde: A mixture of 8-bromo-2-methyl-6-(trifluoromethyl)quinoline (4.1 g, 14 mmol), and selenium oxide (2.0 g, 18 mmol) were added to 400 mL of dioxane and 3 mL of water and heated to reflux overnight. The following day, the reaction was cooled and the selenium was filtered off, the filtrate concentrated to dryness, and chloroform was added. Solids were filtered away again, and the filtrate was purified by silica gel flash chromatography using hexane-ethyl acetate (eluent) to yield the desired product (3.0 g, 66% yield). MS APCI (+) m/z 303.1 (M−1) detected.

Step C: Preparation of 8-bromo-2-(2-methoxyvinyl)-6-(trifluoromethyl)quinoline: To 40 mL of dry THF was added (methoxymethyl)triphenylphosphonium chloride (3.7 g, 11 mmol) and cooled to 0° C. in an ice bath. Potassium 2-methylpropan-2-olate (12 mL, 12 mmol) was next added slowly and the reaction was stirred for 30 minutes. 8-Bromo-6-(trifluoromethyl)quinoline-2-carbaldehyde (3.0 g, 9.9 mmol) dissolved in 6 mL was added slowly and the reaction was stirred overnight, warming to ambient temperature. The following day, the reaction was concentrated, the solid triturated in diethyl ether and solids were removed by filtration, to isolate a viscous material that was taken directly on to next step without further purification.

Step D: Preparation of 8-bromo-2-(7-(2-methoxyethoxy)imidazo[1,2-a]pyridin-3-yl)-6-(trifluoromethyl)quinoline: To 60 mL of THF and 12 mL of water was added 8-bromo-2-(2-methoxyvinyl)-6-(trifluoromethyl)quinoline (3.3 g, 9.9 mmol) and 1-bromopyrrolidine-2,5-dione (1.95 g, 10.9 mmol) and the reaction was stirred for 4 hours at ambient temperature. Next, 5-(2-methoxyethoxy)pyridine-2-amine (1.67 g, 9.9 mmol) was added and the reaction was refluxed overnight. The next day, the reaction was concentrated and purified on silica gel using 6% ammonium hydroxide in methanol and chloroform (eluent) to yield desired product contaminated with ±50% triphenylphosphine oxide(s) (0.200 g, 2.2% yield). Material was taken on to the next step without purification. MS APCI (+) m/z 466.1/468.1 (M+1/+3) detected.

Step E: Preparation of tert-butyl 1-(2-(7-(2-methoxyethoxy)imidazo[1,2-a]pyridin-3-yl)-6-(trifluoromethyl)quinolin-8-yl)piperidin-4-ylcarbamate: To a sealed vial containing 2-3 mL of dry, deoxygenated toluene was added 8-bromo-2-(7-(2-methoxyethoxy)imidazo[1,2-a]pyridin-3-yl)-6-(trifluoromethyl)quinoline (0.10 g, 0.21 mmol), tert-butyl piperidine-4-ylcarbamate (0.056 g, 0.28 mmol), cesium carbonate (0.10 g, 0.32 mmol), Pd₂(dba)₃ (0.019 g, 0.021 mmol) and rac-BINAP (0.027 g, 0.042 mmol), and the reaction was heated to 95° C. overnight. Minimal conversion was observed, so reaction was recharged with an additional 1 full equivalent each of Pd₂(dba)₃ and rac-BINAP and reaction again heated to 95° C. overnight. The following day, the reaction was concentrated and purified using silica gel and 6% ammonium hydroxide in methanol and chloroform (eluent) to yield desired product contaminated by small amounts of triphenylphosphine oxides (0.120 g, 95% yield). Material was taken on to the next step without purification. MS APCI (+) m/z 586.1 (M+1) detected.

Step F: Preparation of 1-(2-(7-(2-methoxyethoxy)imidazo[1,2-a]pyridin-3-yl)-6-(trifluoromethyl)quinolin-8-yl)piperidin-4-amine: To a flask was added tert-butyl 1424742-methoxyethoxy)imidazo[1,2-a]pyridin-3-yl)-6-(trifluoromethyl)quinolin-8-yl)piperidin-4-ylcarbamate (0.100 g, 0.17 mmol) and a mixture of 1-1 trifluoroacetic acid and dichloromethane, and the reaction was stirred for 2 hours. The reaction was concentrated and purified by silica gel flash chromatography eluting with 6% ammonium hydroxide in methanol and chloroform (eluent) to yield desire material (0.013 g, 0.027 mmol, 16% yield). MS APCI (+) m/z 486.2 (M+1) detected.

### Example 59

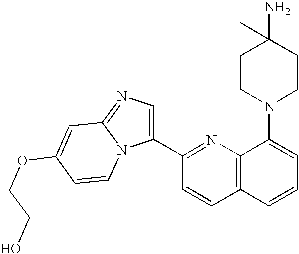

### 2-(3-(8-(4-amino-4-methylpiperidin-1-yl)quinolin-2-yl)imidazo[1,2-a]pyridin-7-yloxy)ethanol

To a 25 mL flask containing 1-(2-(7-(2-methoxyethoxy)imidazo[1,2-a]pyridin-3-yl)quinolin-8-yl)-4-methylpiperidin-4-amine (0.050 g, 0.12 mmol) was added CH₂Cl₂ (12 mL) and the solution was cooled to −78° C. BBr₃ (1.0 M in CH₂Cl₂, 0.58 ml, 0.58 mmol) was added dropwise and the reaction stirred at −78° C. for 1 hour, then slowly warmed to 0° C. over 2.0 hours, and then warmed to ambient temperature and stirred for 1.0 hour. The reaction was quenched by the addition of a saturated aqueous Na₂CO₃ solution (10 mL) and the layers were separated. The aqueous phase was extracted with 10% IPA/CH₂Cl₂ with small amount of MeOH to completely dissolve the solids (3×10 mL) and the combined organic phases were dried over Na₂SO₄. The mixture was filtered and concentrated in vacuo and purified via column chromatography (6% NH₄OH in MeOH)/CH₂Cl₂, 2% to 20%) to afford 0.040 g (83%) of the title compound as a solid. MS APCI (+) m/z 418.1 [M+H]⁺ detected.

### Example 60

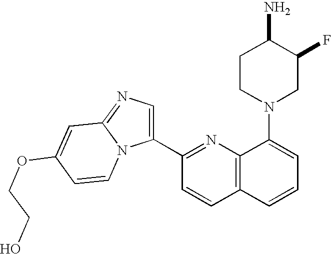

### 2-(3-(8-(cis-4-amino-3-fluoropiperidin-1-yl)quinolin-2-yl)imidazo[1,2-a]pyridin-7-yloxy)ethanol

Prepared according to Example 59 using cis-3-fluoro-1-(2-(7-(2-methoxyethoxy)imidazo[1,2-a]pyridin-3-yl)quinolin-8-yl)piperidin-4-amine in place of 1-(2-(7-(2-methoxyethoxy)imidazo[1,2-a]pyridin-3-yl)quinolin-8-yl)-4-methylpiperidin-4-amine MS APCI (+) m/z 422.3 [M+H]⁺ detected.

### Example 61

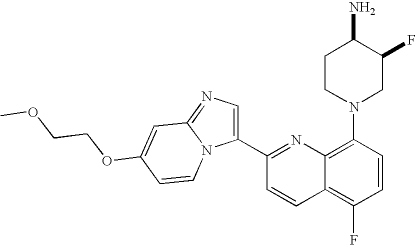

### cis-3-Fluoro-1-(5-fluoro-2-(7-(2-methoxyethoxy)imidazo[1,2-a]pyridin-3-yl)quinolin-8-yl)piperidin-4-amine

Step A: Preparation of Benzyl-cis-3-fluoro-1-(4-fluoro-2-nitrophenyl)piperidin-4-ylcarbamate: To a round bottom flask was added 1,4-difluoro-2-nitrobenzene (1.18 ml, 10.9 mmol) which was dissolved in 2-propanol (20 mL). To this solution was added NEt₃ (3.45 ml, 24.8 mmol) followed by benzyl cis-3-fluoropiperidin-4-ylcarbamate (2.5 g, 9.9 mmol) in one portion. The suspension was warmed to 75° C. and stirred for 15 hours. The reaction mixture was cooled to ambient temperature and diluted with diethyl ether (200 mL) and CH₂Cl₂ (50 mL). The solution was washed with 1 N HCl (2×50 mL). The organic layer was washed with a saturated aqueous NaHCO₃ solution and brine, dried over Na₂SO₄, filtered and concentrated. The crude solid was slurried in hexanes (50 mL) and the solid was collected by filtration. The solid was washed with hexanes (3×50 mL). This provided 2.74 g (71%) of the title compound as a solid which was sufficiently pure to be carried on to the next step. MS APCI (+) m/z 391.0 (M+1) detected.

Step B: Preparation of Benzyl 1-(2-amino-4-fluorophenyl)-cis-3-fluoropiperidin-4-ylcarbamate: To a round bottom flask was added benzyl cis-3-fluoro-1-(4-fluoro-2-nitrophenyl)piperidin-4-ylcarbamate (2.0 g, 5.1 mmol) which was dissolved in THF (100 mL), water (18 mL) and MeOH (18 mL). To this solution was added Fe (0) (7.13 g, 128 mmol) as a powder followed by 1.0 N HCl (4.4 mL). The mixture was stirred at ambient temperature for 20 hours. The mixture was filtered through Celite® and the Celite was washed with CHCl₃ (200 mL). The filtrate was washed with saturated aqueous NaHCO₃ and brine, dried over MgSO₄, filtered and concentrated, affording 1.85 grams (quantitative yield) of the desired product as a solid.

Step C: Preparation of cis-3-fluoro-1-(5-fluoro-2-methylquinolin-8-yl)piperidin-4-amine: Benzyl 1-(2-amino-4-fluorophenyl)-cis-3-fluoropiperidin-4-ylcarbamate (305 mg, 0.85 mmol) was weighed into a flask and dissolved in 10 mL of 6 N HCl. The reaction mixture was heated to reflux, followed by drop-wise addition of (E)-but-2-enal (147 μL, 1.77 mmol) over 25 minutes. Following complete addition the reaction was heated at 100° C. for an additional 35 minutes. The reaction was cooled to ambient temperature, followed by addition of 20 mL of Et₂O. The reaction was stirred for 5 minutes followed by removal of Et₂O by separatory funnel. The aqueous layer was placed into the original reaction flask and ZnCl₂ (115 mg, 0.85 mmol) was added in two portions followed by cooling to 0° C. The aqueous layer was taken to pH=8.0 using concentrated NH₄OH. The aqueous layer was extracted with Et₂O and EtOAc. The combined organic layers were dried over Na₂SO₄, filtered, and concentrated in vacuo, affording the desired product as a solid. MS APCI (+) m/z 278.2 (M+1) detected.

Step D: Preparation of tert-butyl cis-3-fluoro-1-(5-fluoro-2-methylquinolin-8-yl)piperidin-4-ylcarbamate: Dissolve cis-3-fluoro-1-(5-fluoro-2-methylquinolin-8-yl)piperidin-4-amine in dichloromethane. Treat with three equal portions with di-tert-butyl dicarbonate. After the additions, allow the solution to stir at ambient temperature for 14 hours. Then wash the solution three times with saturated aqueous NaHCO₃, dry the organic phase over Na₂SO₄, filter and concentrate in vacuo to provide the desired product.

Step E: Preparation of tert-butyl cis-1-(2-(dibromomethyl)-5-fluoroquinolin-8-yl)-3-fluoropiperidin-4-ylcarbamate: Prepare according to Example 26 step B: Place tert-butyl trans-3-fluoro-1-(5-fluoro-2-methylquinolin-8-yl)piperidin-4-ylcarbamate into a flask and add NaOAc. Suspend the solids in HOAc, and heat the mixture to 70° C. Add bromine (as a solution in HOAc) dropwise over 25 minutes. Following complete addition, heat the mixture to 100° C. for 1 hour. Cool the reaction to ambient temperature, then pour into crushed ice. Once the ice has melted, extract the mixture with EtOAc. Dry the combined organic phase over MgSO₄, filter, and concentrate.

Step F: Preparation of ethyl 8-(cis-4-(tert-butoxycarbonylamino)-3-fluoropiperidin-1-yl)-5-fluoroquinoline-2-carboxylic acid: Prepare according to Example 26 step C: Place tert-butyl cis-1-(2-(dibromomethyl)-5-fluoroquinolin-8-yl)-3-fluoropiperidin-4-ylcarbamate into a round bottom flask and add EtOH, followed by silver nitrate in a 1:1 mixture of EtOH/H₂O. Heat the mixture to reflux for 1 hour. Filter the hot mixture through a medium frit sintered glass funnel to remove the carboxylic acid analog. Concentrate the mother liquor, add water and extract with EtOAc. Dry the combined organic phases over Na₂SO₄, filter and concentrate to afford the desired product.

Step G: Preparation of tert-butyl cis-3-fluoro-1-(5-fluoro-2-dihydroxymethyl)quinolin-8-yl)piperidin-4-ylcarbamate: Prepare according to Example 26 step D: Place ethyl 8-(cis-4-(tert-butoxycarbonylamino)-3-fluoropiperidin-1-yl)-5-fluoroquinoline-2-carboxylate into a round bottom flask and dissolve in CH₂Cl₂. Cool the solution to −78° C., and add DIBAL-H dropwise over 10 minutes. Allow the solution warm to ambient temperature with stirring over 2 hours. Quench the reaction with MeOH, and then add Rochelle's Salts and stir the resulting mixture overnight. Partition the mixture between with ethyl acetate and water, and concentrate the organic phase to afford the desired product. The desired product may be further purified by flash column chromatography.

Step H: Preparation of tert-butyl cis-3-fluoro-1-(5-fluoro-2-formylquinolin-8-yl)piperidin-4-ylcarbamate: Prepare according to Example 26 step E: Place tert-butyl cis-3-fluoro-1-(5-fluoro-2-(hydroxymethyl)quinolin-8-yl)piperidin-4-ylcarbamate and DMSO into a flask and add CH₂Cl₂, then cool to 0° C. Add pyridine sulfur trioxide and stir for 1 hour at 0° C. Pour the solution into water and extract with ethyl acetate. Combine the organic fractions and dry over MgSO₄, then filter and concentrate in vacuo to afford the desired product.

Step I: Preparation of tert-butyl cis-3-fluoro-1-(5-fluoro-2-(2-methoxyvinyl)quinolin-8-yl)piperidin-4-ylcarbamate: Prepare according to Example 26 step F: Place (methoxymethyl)triphenylphosphonium chloride into a round bottom flask and add THF. Cool to 0° C., and add KOtBu dropwise. Stir for 15 minutes at ambient temperature, then add tert-butyl cis-3-fluoro-1-(5-fluoro-2-formylquinolin-8-yl)piperidin-4-ylcarbamate dropwise as a solution in THF over 3 minutes. Stir the reaction at ambient temperature for 12 hours. Concentrate in vacuo, and further purify the crude residue by flash column chromatography to afford the desired product.

Step J: Preparation of tert-butyl cis-3-fluoro-1-(5-fluoro-2-(7-(2-methoxyethoxy)imidazo[1,2-a]pyridin-3-yl)quinolin-8-yl)piperidin-4-ylcarbamate: Prepare according to Example 26 step G: Dissolve tert-butyl cis-3-fluoro-1-(5-fluoro-2-(2-methoxyvinyl)quinolin-8-yl)piperidin-4-ylcarbamate in THF and de-ionized water and add N-bromosuccinimide. When analysis (for example TLC and/or LC/MS) indicates complete consumption of the starting material, add 4-(2-Methoxyethoxy)pyridin-2-amine and heat to reflux for 10 hours. Concentrate the crude reaction mixture to afford a crude residue which may be further purified by flash column chromatography.

Step K: Preparation of cis-3-fluoro-1-(5-fluoro-2-(7-(2-methoxyethoxy)imidazo[1,2-a]pyridin-3-yl)quinolin-8-yl)piperidin-4-amine: Prepare according to Example 26 step I: Remove the Boc group with TFA in CH₂Cl₂ to afford the desired product. The product may be further purified by flash column chromatography.

### Example 62

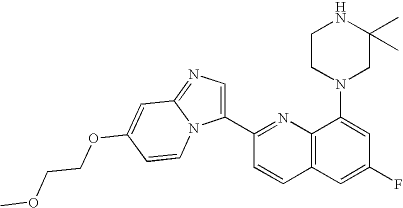

### 8-(3,3-dimethylpiperazin-1-yl)-6-fluoro-2-(7-(2-methoxyethoxy)imidazo[1,2-a]pyridin-3-yl)quinoline

Step 1A: Preparation of 8-bromo-6-fluoro-2-methylquinoline: 2-Bromo-4-fluorobenzenamine (10 g, 52.6 mmol) was weighed into a 100 mL flask and dissolved in 40 mL of 6 N HCl. The reaction mixture was heated to reflux, followed by dropwise addition of (E)-but-2-enal (4.578 ml, 55.3 mmol) mixed with 1.0 mL deionized water over 25 minutes. Following complete addition the reaction was heated at 100° C. for an additional 35 minutes. The reaction was cooled to ambient temperature, followed by addition of 50 mL of Et₂O. The reaction was stirred for 5 minutes followed by removal of Et₂O by partitioning. The aqueous layer was replaced into the original reaction flask and ZnCl₂ (3.5865 g, 26.3 mmol) was added in two portions followed by cooling to 0° C. over 30 minutes. The pH of the crude reaction mixture was adjusted to pH=8.0 using concentrated NH₄OH. The crude mixture was extracted with Et₂O, followed by ethyl acetate. The combined organics were then dried over Na₂SO₄, filtered and concentrated in vacuo, affording the desired product as a solid. (10.7 g, 85% yield) MS APCI (+) m/z 240.2 and 242.2 (M+1 of each isotope) detected.

Step 1B: Preparation of 8-bromo-2-(dibromomethyl)-6-fluoroquinoline: 8-Bromo-6-fluoro-2-methylquinoline (10.7 g, 44.6 mmol) was weighed into a 1000 mL flask, followed by addition of NaOAc (21.9 g, 267 mmol). The solids were suspended in 500 mL of AcOH, and the reaction heated to 70° C. Bromine (6.85 mL, 134 mmol) was added dropwise over 25 minutes as a solution in 30 mL of AcOH. Following complete addition, the reaction was stirred at 100° C. for 1 hour. The reaction was cooled to ambient temperature, then poured onto 750 cc of ice. The ice was allowed to melt completely and the slurry was separated by partitioning into ethyl acetate. The combined organics were dried over magnesium sulfate, filtered and concentrated in vacuo to afford a solid. (17.2 g, 97% yield).

Step 1C: Preparation of 8-bromo-6-fluoroquinoline-2-carboxylate and 8-bromo-6-fluoroquinoline-2-carboxylic acid: 8-Bromo-2-(dibromomethyl)-6-fluoroquinoline (17.2 g, 43.2 mmol) was weighed into a flask and dissolved in 250 mL of EtOH, followed by addition of silver nitrate (23.5 g, 138 mmol) in 100 mL of 1:1 EtOH/H₂O. The reaction was heated to reflux for 1 hour, then filtered hot through a medium flit sintered glass funnel, affording 5.84 g of 8-bromo-6-fluoroquinoline-2-carboxylic acid. The mother liquor was concentrated in vacuo, followed by extractive work-up (200 mL ethyl acetate/water), then washed with ethyl acetate. The combined organics were dried over Na₂SO₄, filtered and concentrated in vacuo to afford ethyl 8-bromo-6-fluoroquinoline-2-carboxylate as a semi-solid (99% overall (6.4 g and 5.8 g respectively)). MS APCI (+) m/z 298 and 300 (M+1 of each isotope) detected; MS APCI (−) m/z 268 and 269.9 (M−1 of each isotope) detected.

Step 1D: Preparation of (8-bromo-6-fluoroquinolin-2-yl)methanol: Ethyl 8-bromo-6-fluoroquinoline-2-carboxylate (3.201 g, 10.7 mmol) was weighed into a flask and dissolved in 100 mL of DCM. The reaction was cooled to −78° C., followed by dropwise addition of DIBAL-H (21.48 ml, 32.22 mmol) over 10 minutes. The reaction was allowed to stir and warm to ambient temperature over 2 hours. The reaction was quenched with 10 mL MeOH, followed by addition of 100 mL of Rochelle's Salts, and stirred overnight. The reaction was partitioned with ethyl acetate, and the organic fractions were combined and concentrated in vacuo. The crude semi-solid was purified by flash column chromatography (eluting with a 20-50% ethyl acetate/Hexane gradient), affording the desired product as a semi-solid (2.27 g, 42% yield). MS APCI (+) m/z 256.1 and 258 (M+1 of each isotope) detected.

Step 1E: Preparation of 8-bromo-6-fluoroquinoline-2-carbaldehyde: (8-Bromo-6-fluoroquinolin-2-yl)methanol (2 g, 7.8 mmol), DMSO (8.9 ml, 125.0 mmol), and triethylamine (4.9 ml, 35 mmol) were weighed into a 100 mL flask and dissolved in a 10 mL of DCM, followed by cooling to 0° C. Pyridine sulfur trioxide (4.351 g, 27.3 mmol) was added and the reaction was stirred at 0° C. for 1 hour. The reaction was poured into 50 mL water and extracted with ethyl acetate. The combined organics were dried over MgSO₄, filtered and concentrated in vacuo affording a semi-solid, which was further purified by triturating with 20% ethyl acetate/Hexane, affording the desired product as a solid (1.35 g, 68% yield).

Step 1F: Preparation of 8-bromo-6-fluoro-2-(2-methoxyvinyl)quinoline: (Methoxymethyl)triphenylphosphonium chloride (1.5 g, 4.3 mmol) was weighed into a 50 mL flask and dissolved in 40 mL of anhydrous THF. The reaction was cooled to 0° C., followed by dropwise addition of KOtBu (4.7 ml, 4.7 mmol). The reaction was allowed to stir for 15 minutes at 23° C., followed by dropwise addition of 8-bromo-6-fluoroquinoline-2-carbaldehyde (1.0 g, 3.9 mmol) as a solution in 10 mL of THF over 3 minutes. The reaction was allowed to stir at ambient temperature for 12 hours. The crude reaction was concentrated in vacuo, followed by trituration with Et₂O, and ethyl acetate, affording the desired product as a solid (900 mg, 82% yield) MS APCI (+) m/z 282.2 and 284 (M+1 of each isotope) detected.

Step 2A: Preparation of 2-chloro-4-(2-methoxyethoxy)pyridine: A mixture of 2-chloro-4-nitropyridine (43.6 g, 275 mmol) and 2-methoxyethanol (325 ml, 425 mmol) was cooled to 0° C. Potassium 2-methylpropan-2-olate (35.7 g, 302 mmol) was added and the resulting mixture was stirred while warming to ambient temp over 2 hours. The reaction mixture was concentrated under reduced pressure followed by dilution with 500 ml of water. The resulting mixture was extracted with dichloromethane. The combined organic layers were dried over MgSO₄ and concentrated under reduced pressure to produce the desired compound as an oil. (50.2 g) MS APCI (+) m/z 188 and 189.9 (M+1 of each isotope) detected.

Step 2B: Preparation of 4-(2-methoxyethoxy)pyridin-2-amine: A steady stream of nitrogen was passed through a mixture of 2-chloro-4-(2-methoxyethoxy)pyridine (50.1 g, 267 mmol), Pd₂ dba₃ (4.89 g, 5.34 mmol), XPHOS (5.09 g, 10.7 mmol) and tetrahydrofuran (445 ml) for 10 minutes. To the resulting degassed mixture was added lithium bis(trimethylsilyl)amide (561 ml, 561 mmol). After addition, the resulting mixture was heated to 60° C. for 18 hours. The reaction was cooled to ambient temperature and diluted with 1 N hydrochloric acid (200 mL). The resulting solution was washed with methyl-tert-butyl ether. The pH of the aqueous layer was adjusted to 11 with 6 N NaOH and extracted with dichloromethane. The combined organic layers were dried over MgSO₄ and concentrated under reduced pressure to yield title compound. (35 g) MS APCI (+) m/z 169 (M+1) detected.

Step 2C: Preparation of 8-bromo-6-fluoro-2-(7-(2-methoxyethoxy)-imidazo[1,2-a]pyridin-3-yl)quinoline: 8-Bromo-6-fluoro-2-(2-methoxyvinyl)quinoline (900 mg, 3.19 mmol) was dissolved in a solution of 20 mL of THF and 4 mL of deionized water. N-Bromosuccinimide (596 mg, 3.35 mmol) was added and the reaction monitored by TLC/LC for complete conversion to the alpha-bromo aldehyde. 4-(2-Methoxyethoxy)pyridin-2-amine (537 mg, 3.19 mmol) was added, and the reaction heated to reflux for 10 hours. The crude reaction mixture was concentrated in vacuo affording a solid which was triturated successively with ethyl acetate and Et₂O, followed by trituration with a 1:1 mixture of Et₂O and DCM, to afford the desired product as powder (746 mg, 56% yield). MS APCI (+) m/z 416.2 and 418.1 (M+1 of each isotope) detected.

Step 3A: Preparation of tert-butyl 4-(6-fluoro-2-(7-(2-methoxyethoxy)imidazo[1,2-a]pyridin-3-yl)quinolin-8-yl)-2,2-dimethylpiperazine-1-carboxylate: tert-Butyl 2,2-dimethylpiperazine-1-carboxylate (0.050 g, 0.23 mmol), 8-bromo-6-fluoro-2-(7-(2-methoxyethoxy)imidazo[1,2-a]pyridin-3-yl)quinoline (0.075 g, 0.18 mmol), micronized Cs₂CO₃ (0.082 g, 0.25 mmol), Binap-racemic (0.022 g, 0.036 mmol) and Pd₂ dba₃ (0.016 g, 0.018 mmol) were combined in toluene (1 mL). The solution was degassed with argon and then heated to reflux under argon for 14 hours. The reaction was cooled and loaded onto a column of SiO₂ and eluted with a gradient from 1-20% (6% NH₄OH in MeOH)/ethyl acetate, (21.2 mg). MS APCI (+) m/z 550.1 (M+1) detected.

Step 3B: Preparation of 8-(3,3-dimethylpiperazin-1-yl)-6-fluoro-2-(7-(2-methoxyethoxy)imidazo[1,2-a]pyridin-3-yl)quinoline: tert-Butyl 4-(6-fluoro-2-(7-(2-methoxyethoxy)imidazo pyridin-3-yl)quinolin-8-yl)-2,2-dimethylpiperazine-1-carboxylate (0.021 g, 0.039 mmol) was dissolved in MeOH (0.25 mL) and treated with 4 M hydrogen chloride in dioxane (0.24 ml, 0.96 mmol). The reaction was stirred at ambient temperature for 5 hours. The mixture was concentrated in vacuo, re-dissolved in MeOH and re-concentrated three times. The residue was purified by chromatography on SiO₂, eluting with a gradient of (6% NH₄OH in MeOH) in dichloromethane, (12.6 mg). The sample was dissolved in MeOH (1 mL) and treated with 4M HCl in dioxane (1 mL). The mixture was concentrated in vacuo then re-dissolved and re-concentrated in vacuo from MeOH three times and placed under high vacuum for 4 hours. MS APCI (+) m/z 450.3 (M+1) detected.

### Example 63

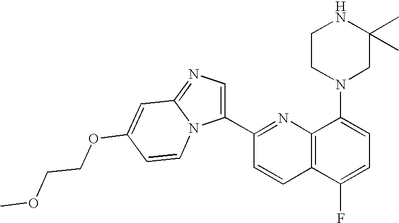

### 8-(3,3-dimethylpiperazin-1-yl)-5-fluoro-2-(7-(2-methoxyethoxy)imidazo[1,2-a]pyridin-3-yl)quinoline

Step 1A: Preparation of 8-bromo-5-fluoro-2-methylquinoline: 2-Bromo-5-fluorobenzenamine (15 g, 78.9 mmol) was weighed into a flask and dissolved in 100 mL of 6N HCl. The reaction mixture was heated to reflux, followed by dropwise addition of (E)-but-2-enal (6.87 ml, 83 mmol) mixed with 1.0 mL deionized water over 25 minutes. Following complete addition, the reaction was heated at 100° C. for an additional 35 minutes. The reaction was cooled to ambient temperature, followed by addition of 50 mL of Et₂O. The reaction was stirred for 5 minutes followed by removal of Et₂O by separatory funnel. ZnCl₂ (3.587 g, 26 mmol) was added to the aqueous layer in two portions and the reaction mixture was cooled to 0° C. over 30 minutes. The aqueous layer was adjusted to pH 8.0 using concentrated NH₄OH. The aqueous layer was then extracted with Et₂O and then with EtOAc. The combined organic phases were dried over Na₂SO₄, filtered and concentrated in vacuo, affording the desired product 8-bromo-5-fluoro-2-methylquinoline (18.1 g) as a solid.

Step 1B: Preparation of 8-bromo-2-(dibromomethyl)-5-fluoroquinoline: 8-Bromo-5-fluoro-2-methylquinoline (18.1 g, 75.4 mmol) was weighed into a 1000 mL flask, followed by addition of NaOAc (37.1 g, 452 mmol). The solids were suspended in 500 mL of AcOH, and the reaction was heated to 70° C. Bromine (11.6 ml, 226 mmol) was added dropwise over 25 minutes as a solution in 50 mL of AcOH. Following complete addition, the reaction was stirred at 100° C. for 1 hour. The reaction was cooled to ambient temperature, then poured onto 700 cc of ice. The ice was allowed to melt completely and the mixture was extracted with ethyl acetate. The combined organic phases were dried over Na₂SO₄, filtered, concentrated in vacuo and dried under vacuum, affording the desired product (27 g, 90%).

Step 1C: Preparation of 8-bromo-5-fluoroquinoline-2-carboxylate and 8-bromo-5-fluoroquinoline-2-carboxylic acid: 8-Bromo-2-(dibromomethyl)-5-fluoroquinoline (25 g, 63 mmol) was weighed into a flask and dissolved in 250 mL of EtOH, followed by addition of silver nitrate (34 g, 201 mmol) in 100 mL of 1:1 EtOH/H₂O. The reaction was heated to reflux for 1 hour, then filtered hot through a medium flit sintered glass funnel, affording 2.17 g of a powder. The mother liquor was concentrated in vacuo, followed by extractive work-up (500 mL EtOAc/water). The combined organic phases were dried over Na₂SO₄ and concentrated in vacuo to afford ethyl 8-bromo-5-fluoroquinoline-2-carboxylate (9.3 g, 99% yield) and 8-bromo-5-fluoroquinoline-2-carboxylic acid (8 g, 94% yield).

Step 1D: Preparation of (8-bromo-5-fluoroquinolin-2-yl)methanol: Ethyl 8-bromo-5-fluoroquinoline-2-carboxylate (5.52 g, 18.5 mmol) was weighed into a flask and dissolved in 400 mL of DCM. The reaction was cooled to −78° C., followed by dropwise addition of DIBAL-H (49.4 ml, 74.1 mmol) over 10 minutes. The reaction was allowed to stir and warm to ambient temperature over 2 hours. The reaction was quenched with 10 mL MeOH and 100 mL of 1 N Rochelle's salt and stirred overnight. The aqueous layer was extracted with EtOAc, followed by concentration in vacuo. The residue was purified by flash column chromatography (20-50% EtOAc/Hex), affording the desired product as a solid (2.25 g). MS APCI (+) m/z 256.1 (M+1) detected.

Step 1E: Preparation of 8-bromo-5-fluoroquinoline-2-carbaldehyde: (8-Bromo-5-fluoroquinolin-2-yl)methanol (1.85 g, 7.22 mmol), DMSO (8.20 ml, 116 mmol) and triethylamine (4.53 ml, 32.5 mmol) were weighed into a 100 mL flask and dissolved in a 1:1 mixture of DCM/DMSO, followed by cooling to 0° C. Pyridine sulfur trioxide (4.02 g, 25.3 mmol) was added and the reaction was stirred at 0° C. for 1 hour. The reaction was poured into 50 mL water and extracted with EtOAc. The combined organic phases were dried over MgSO₄, filtered, and concentrated in vacuo affording a semi-solid, which was further purified by flash column chromatography 10-40% EtOAc/Hexane, affording 8-bromo-5-fluoroquinoline-2-carbaldehyde (1.71 g).

Step 1F: Preparation of 8-bromo-5-fluoro-2-(2-methoxyvinyl)quinoline: (Methoxymethyl)triphenylphosphonium chloride (1.9 g, 5.6 mmol) was weighed into a flask and dissolved in 40 mL of anhydrous THF. The reaction was cooled to 0° C., followed by dropwise addition of KOtBu (6.1 ml, 6.1 mmol). The reaction was stirred for 15 minutes at ambient temperature, followed by dropwise addition of 8-bromo-5-fluoroquinoline-2-carbaldehyde (1.3 g, 5.1 mmol) as a solution in 10 mL of THF over 3 minutes, affording an immediate dark red/brown color change. The reaction was allowed to stir at ambient temperature for 12 hours. The reaction was concentrated in vacuo, followed by trituration with Et₂O, then ethyl acetate, to afford the crude desired product, which was used in the next step without purification.

Step 2A: Preparation of 2-chloro-4-(2-methoxyethoxy)pyridine: A mixture of 2-chloro-4-nitropyridine (43.6 g, 275 mmol) and 2-methoxyethanol (325 ml, 425 mmol) was cooled to 0° C. Potassium 2-methylpropan-2-olate (35.7 g, 302 mmol) was added and the resulting mixture was stirred while warming to ambient temperature over 2 hours. The reaction mixture was concentrated under reduced pressure followed by dilution with 500 ml of water. The resulting mixture was extracted with dichloromethane. The combined organic layers were dried over MgSO₄ and concentrated under reduced pressure to produce the desired compound as an oil (50.2 g). MS APCI (+) m/z 188 and 189.9 (M+1 of each isotope) detected.

Step 2B: Preparation of 4-(2-methoxyethoxy)pyridin-2-amine: A steady stream of nitrogen was passed through a mixture of 2-chloro-4-(2-methoxyethoxy)pyridine (50.1 g, 267 mmol), Pd₂ dba₃ (4.89 g, 5.34 mmol), XPHOS (5.09 g, 10.7 mmol) and tetrahydrofuran (445 ml) for 10 minutes. To the resulting degassed mixture was added lithium bis(trimethylsilyl)amide (561 ml, 561 mmol). After addition, the resulting mixture was heated to 60° C. for 18 hours. The reaction was cooled to ambient temperature and diluted with 1 N hydrochloric acid (200 mL). The resulting solution was washed with methyl-tert-butyl ether. The pH of the aqueous layer was adjusted to 11 with 6 N NaOH and extracted with dichloromethane. The combined organic layers were dried over MgSO₄ and concentrated under reduced pressure to yield title compound (35 g). MS APCI (+) m/z 169 (M+1) detected.

Step 3A: Preparation of 8-bromo-5-fluoro-2-(7-(2-methoxyethoxy)-imidazo[1,2-a]pyridin-3-yl)quinoline: 8-Bromo-5-fluoro-2-(2-methoxyvinyl)quinoline (2.4 g, 8.5 mmol) was dissolved in a solution of 20 mL of THF and 4 mL of deionized water. N-Bromosuccinimide (1.59 g, 8.9 mmol) was added and the reaction was stirred for 2 hours. 4-(2-methoxyethoxy)pyridin-2-amine (1.43 g, 8.51 mmol) was added, and the reaction was heated to reflux for 10 hours. The crude reaction mixture was concentrated in vacuo affording a solid which was triturated successively with EtOAc and Et₂O, followed by trituration with a mixture of Et₂O and CH₂Cl₂ to afford 8-bromo-5-fluoro-2-(7-(2-methoxyethoxy)imidazo[1,2-a]pyridin-3-yl)quinoline (746 mg) as a solid. MS APCI (+) m/z 416.2 (M+1) detected.

Step 4A: Preparation of tert-butyl 4-(5-fluoro-2-(7-(2-methoxyethoxy)imidazo[1,2-a]pyridin-3-yl)quinolin-8-yl)-2,2-dimethylpiperazine-1-carboxylate: tert-Butyl 2,2-dimethylpiperazine-1-carboxylate (0.106 g, 0.494 mmol), 8-bromo-5-fluoro-2-(7-(2-methoxyethoxy)imidazo[1,2-a]pyridin-3-yl)quinoline (0.137 g, 0.329 mmol), micronized Cs₂CO₃ (0.15 g, 0.46 mmol), Binap-racemic (0.041 g, 0.066 mmol) and Pd₂ dba₃ (0.030 g, 0.033 mmol) were combined in toluene (2 mL). The solution was degassed with argon and then heated to reflux under argon for 14 hours. The reaction mixture was briefly cooled and treated with additional tert-butyl 2,2-dimethylpiperazine-1-carboxylate (0.106 g, 0.494 mmol), Binap-racemic (0.041 g, 0.066 mmol), and Pd₂ dba₃ (0.030 g, 0.033 mmol). The flask degassed with argon and then heated to reflux under argon for 14 hours. MS APCI (+) m/z 550.1 (M+1) detected. The desired product is purified by chromatography on SiO₂ eluting with a gradient of 1-20% (6% NH₄OH in MeOH)/ethyl acetate.

Step 4B: Preparation of 8-(3,3-dimethylpiperazin-1-yl)-5-fluoro-2-(7-(2-methoxyethoxy)imidazo[1,2-a]pyridin-3-yl)quinoline: tert-Butyl 4-(5-fluoro-2-(7-(2-methoxyethoxy)imidazo[1,2-a]pyridin-3-yl)quinolin-8-yl)-2,2-dimethylpiperazine-1-carboxylate is dissolved in dioxane and treated with 4 M HCl in dioxane (25 eq.). The reaction mixture is stirred until consumption of the starting material is complete. The reaction mixture is concentrated in vacuo, re-suspended in MeOH and re-concentrated three times. This crude product is purified by chromatography on silica gel, eluting with a gradient of 1-20% (6% NH₄OH in MeOH)/methylene chloride.

### Example 64

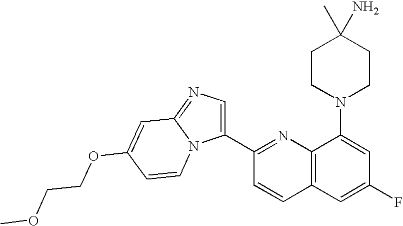

### 1-(6-fluoro-2-(7-(2-methoxyethoxy)imidazo[1,2-a]pyridin-3-yl)quinolin-8-yl)-4-methylpiperidin-4-amine

Step 1A: Preparation of 8-bromo-6-fluoro-2-methylquinoline: 2-Bromo-4-fluorobenzenamine (10 g, 52.6 mmol) was weighed into a flask and dissolved in 40 mL of 6N HCl. The reaction mixture was heated to reflux, followed by dropwise addition of (E)-but-2-enal (4.578 ml, 55.3 mmol) mixed with 1.0 mL deionized water over 25 minutes. Following complete addition the reaction was heated at 100° C. for an additional 35 minutes. The reaction was cooled to ambient temperature, followed by addition of 50 mL of Et₂O. The reaction was stirred for 5 minutes followed by removal of Et₂O by partitioning. The aqueous layer was placed into the original reaction flask and ZnCl₂ (3.5865 g, 26.3 mmol) was then added in two portions followed by cooling to 0° C. over 30 minutes. The pH of the crude reaction mixture was then adjusted to pH=8.0 using concentrated NH₄OH. The crude mixture was extracted with Et₂O, followed by ethyl acetate. The combined organics were dried over Na₂SO₄, filtered and concentrated in vacuo, affording the desired product as a solid (10.7 g, 85% yield). MS APCI (+) m/z 240.2 and 242.2 (M+1 of each isotope) detected.

Step 1B: Preparation of 8-bromo-2-(dibromomethyl)-6-fluoroquinoline: 8-Bromo-6-fluoro-2-methylquinoline (10.7 g, 44.6 mmol) was weighed into a flask, followed by addition of NaOAc (21.9 g, 267 mmol). The solids were suspended in 500 mL of AcOH, and the reaction was heated to 70° C. Bromine (6.85 mL, 134 mmol) was added dropwise over 25 minutes as a solution in 30 mL of AcOH. Following complete addition, the reaction was stirred at 100° C. for 1 hour. The reaction was cooled to ambient temperature, then poured onto 750 cc of ice. The ice was allowed to melt completely and the slurry was separated by partitioning into ethyl acetate. The combined organics were dried over magnesium sulfate, then filtered and concentrated in vacuo to afford a solid. (17.2 g, 97% yield).

Step 1C: Preparation of 8-bromo-6-fluoroquinoline-2-carboxylate and 8-bromo-6-fluoroquinoline-2-carboxylic acid: 8-Bromo-2-(dibromomethyl)-6-fluoroquinoline (17.2 g, 43.2 mmol) was weighed into a flask and dissolved in 250 mL of EtOH, followed by addition of silver nitrate (23.5 g, 138 mmol) in 100 mL of 1:1 EtOH/H₂O. The reaction was heated to reflux for 1 hour. The reaction was filtered hot through a medium frit sintered glass funnel, affording 5.84 g of 8-bromo-6-fluoroquinoline-2-carboxylic acid. The mother liquor was concentrated in vacuo, followed by extractive work-up (200 mL ethyl acetate/water), then washed with ethyl acetate. The combined organics were dried over Na₂SO₄, filtered and concentrated in vacuo to afford ethyl 8-bromo-6-fluoroquinoline-2-carboxylate as a semi-solid. (99% overall (6.4 g and 5.8 g respectively)). MS APCI (+) m/z 298 and 300 (M+1 of each isotope) detected; MS APCI (−) m/z 268 and 269.9 (M−1 of each isotope) detected.

Step 1D: Preparation of (8-bromo-6-fluoroquinolin-2-yl)methanol: Ethyl 8-bromo-6-fluoroquinoline-2-carboxylate (3.201 g, 10.7 mmol) was weighed into a flask and dissolved in 100 mL of DCM. The reaction was cooled to −78° C., followed by dropwise addition of DIBAL-H (21.48 ml, 32.22 mmol) over 10 minutes. The reaction was allowed to stir and warm to ambient temperature over 2 hours. The reaction was quenched with 10 mL MeOH, followed by addition of 100 mL of Rochelle's Salts, and then stirred overnight. The reaction was partitioned with ethyl acetate, and the organic fractions were combined and concentrated in vacuo. The crude semi-solid was purified by flash column chromatography (eluting with a 20-50% ethyl acetate/Hexane gradient), affording the desired product as a semi-solid (2.27 g, 42% yield). MS APCI (+) m/z 256.1 and 258 (M+1 of each isotope) detected.

Step 1E: Preparation of 8-bromo-6-fluoroquinoline-2-carbaldehyde: (8-Bromo-6-fluoroquinolin-2-yl)methanol (2 g, 7.8 mmol), DMSO (8.9 ml, 125.0 mmol), and triethylamine (4.9 ml, 35 mmol) were weighed into a flask and dissolved in a 10 mL of DCM, followed by cooling to 0° C. Pyridine sulfur trioxide (4.351 g, 27.3 mmol) was added and the reaction stirred at 0° C. for 1 hour. The reaction was poured into 50 mL water and extracted with ethyl acetate. The combined organics were dried over MgSO₄, then filtered and concentrated in vacuo affording a semi-solid, which was further purified by triturating with 20% ethyl acetate/Hexane, affording the desired product as a solid (1.35 g, 68% yield).

Step 1F: Preparation of 8-bromo-6-fluoro-2-(2-methoxyvinyl)quinoline: (Methoxymethyl)triphenylphosphonium chloride (1.5 g, 4.3 mmol) was weighed into a 50 mL flask and dissolved in 40 mL of anhydrous THF. The reaction was cooled to 0° C., followed by dropwise addition of KOtBu (4.7 ml, 4.7 mmol). The reaction was allowed to stir for 15 minutes at 23° C., followed by dropwise addition of 8-bromo-6-fluoroquinoline-2-carbaldehyde (1.0 g, 3.9 mmol) as a solution in 10 mL of THF over 3 minutes. The reaction was allowed to stir at ambient temperature for 12 hours. The crude reaction was concentrated in vacuo, followed by trituration with Et₂O, and ethyl acetate, affording the desired product as a solid (900 mg, 82% yield). MS APCI (+) m/z 282.2 and 284 (M+1 of each isotope) detected.

Step 2A: Preparation of 2-chloro-4-(2-methoxyethoxy)pyridine: A mixture of 2-chloro-4-nitropyridine (43.6 g, 275 mmol) and 2-methoxyethanol (325 ml, 425 mmol) was cooled to 0° C. Potassium 2-methylpropan-2-olate (35.7 g, 302 mmol) was added and the resulting mixture was stirred while warming to ambient temperature over 2 hours. The reaction mixture was concentrated under reduced pressure followed by dilution with water. The resulting mixture was extracted with dichloromethane. The combined organic layers were dried over MgSO₄ and concentrated under reduced pressure to produce the desired compound as an oil. (50.2 g) MS APCI (+) m/z 188 and 189.9 (M+1 of each isotope) detected.

Step 2B: Preparation of 4-(2-methoxyethoxy)pyridin-2-amine: A steady stream of nitrogen was passed through a mixture of 2-chloro-4-(2-methoxyethoxy)pyridine (50.1 g, 267 mmol), Pd₂ dba₃ (4.89 g, 5.34 mmol), XPHOS (5.09 g, 10.7 mmol) and tetrahydrofuran (445 ml) for 10 minutes. To the resulting degassed mixture was added lithium bis(trimethylsilyl)amide (561 ml, 561 mmol). After addition, the resulting mixture was heated to 60° C. for 18 hours. The reaction was cooled to ambient temperature and diluted with 1 N hydrochloric acid (200 mL). The resulting solution was washed twice with 500 ml of methyl-tert-butyl ether. The pH of the aqueous layer was adjusted to 11 with 6 N NaOH and extracted with dichloromethane. The combined organic layers were dried over MgSO₄ and concentrated under reduced pressure to yield title compound (35 g). MS APCI (+) m/z 169 (M+1) detected.

Step 2C: Preparation of 8-bromo-6-fluoro-2-(7-(2-methoxyethoxy)-imidazo[1,2-a]pyridin-3-yl)quinoline: 8-Bromo-6-fluoro-2-(2-methoxyvinyl)quinoline (900 mg, 3.19 mmol) was dissolved in a solution of 20 mL of THF and 4 mL of deionized water. N-Bromosuccinimide (596 mg, 3.35 mmol) was added and the reaction monitored by TLC/LC for complete conversion to the alpha-bromo aldehyde. 4-(2-Methoxyethoxy)pyridin-2-amine (537 mg, 3.19 mmol) was added, and the reaction heated to reflux for 10 hours. The crude reaction mixture was concentrated in vacuo affording a solid which was triturated successively with ethyl acetate and Et₂O, followed by trituration with a 1:1 mixture of Eₜ₂O and DCM, to afford the desired product as powder (746 mg, 56% yield). MS APCI (+) m/z 416.2 and 418.1 (M+1 of each isotope) detected.

Step 3A: Preparation of 1-tert-butyl 4-ethyl piperidine-1A-dicarboxylate: The compound was prepared following a procedure outlined in PCT Publication No. WO 01/40217. Ethyl piperidine-4-carboxylate (8.639 ml, 56.10 mmol) was dissolved in dichloromethane (55 mL) and treated in three equal portions with di-tert-butyl dicarbonate (12.24 g, 56.10 mmol). Each addition caused vigorous bubbling and a bit of temperature rise. After the additions, the solution was stirred at ambient temperature for 14 hours. The solution was extracted with saturated NaHCO₃, dried over Na₂SO₄ and concentrated in vacuo to provide the desired product as an oil (14.1 g). ¹H NMR (400 MHz, CDCl₃) δ 4.14 (q, 2H), 4.09-3.95 (brd, 2H), 2.90-2.78 (m, 2H), 2.49-2.38 (m, 1H), 1.92-1.82 (m, 2H), 1.69-1.57 (m, 2H), 1.46 (s, 9H), 1.26 (t, 3H).

Step 3B: Preparation of 1-tert-butyl 4-ethyl 4-methylpiperidine-1,4-dicarboxylate: The compound was prepared following a procedure outlined in PCT Publication No. WO 01/40217. 1-tert-Butyl 4-ethyl piperidine-1,4-dicarboxylate (7.12 g, 27.7 mmol) was dissolved in THF (30 mL) and cooled to −40° C. LHMDS (55.3 ml, 55.3 mmol) was added slowly and the solution was stirred at −40° C. for 1 hour. Iodomethane (3.45 ml, 55.3 mmol) was added and the reaction mixture was warmed to ambient temperature and stirred for 14 hours. The reaction was quenched with water and saturated NaHCO₃. After diluting with methylene chloride, the layers were separated. The aqueous layer was washed twice with methylene chloride, and the combined organic layers were washed with saturated NaCl, dried over Na₂SO₄ and concentrated in vacuo to provide the desired product as an oil (quantitative). ¹H NMR (400 MHz, CDCl₃) δ 4.16 (q, 2H), 3.83-3.70 (m, 2H), 3.03-2.94 (m, 2H), 2.11-2.02 (m, 2H), 1.45 (s, 9H), 1.41-1.30 (m, 2H), 1.26 (t, 3H), 1.20 (s, 3H).

Step 3C: Preparation of 1-(tert-butoxycarbonyl)-4-methylpiperidine-4-carboxylic acid: The compound was prepared following a procedure outlined in PCT Publication No. WO 01/40217. 1-tert-Butyl 4-methylpiperidine-1,4-dicarboxylate (54.2 g, 200 mmol) was dissolved in a solution of EtOH (400 mL) and 2 N NaOH (200 mL). The mixture was heated to 60° C. for 60 hours and then cooled and concentrated in vacuo. The solution was extracted with Et₂O, and the aqueous layer was adjusted to pH 3 with a mixture of concentrated HCl followed by 3 N HCl. The aqueous was extracted with ethyl acetate, and the combined organic layers were washed with saturated NaCl, dried over Na₂SO₄ and concentrated in vacuo to provide the desired product as a solid (45.1 g). ¹H NMR (400 MHz, CDCl₃) δ 3.86-3.67 (brd m, 2H), 3.13-3.01 (m, 2H), 2.12-2.01 (m, 2H), 1.53-1.32 (m, 2H), 1.45 (s, 9H), 1.27 (s, 3H).

Step 3D: Preparation of tert-butyl 4-(benzyloxycarbonylamino)-4-methylpiperidine-1-carboxylate: The compound was prepared following a procedure outlined in Madar, D. J.; et al.; J. Med. Chem. 2006, 49, 6416-6420, and supplementary materials. 1-(tert-Butoxycarbonyl)-4-methylpiperidine-4-carboxylic acid (5.00 g, 20.5 mmol) was dissolved in toluene (40 mL) and treated at ambient temperature with triethylamine (4.30 ml, 30.8 mmol) and diphenylphosphoryl azide (5.98 ml, 27.7 mmol). The reaction was stirred at ambient temperature for 45 minutes then phenylmethanol (10.6 ml, 102 mmol) was added and the mixture was heated to 80° C. for 16 hours. The reaction mixture was concentrated in vacuo. The residue was dissolved in ethyl acetate and washed with saturated NH₄Cl and with saturated NaCl. The organic layer was dried over Na₂SO₄ and concentrated in vacuo to provide the desired product as a semi-solid (25 g), which was utilized in the next step without purification.

Step 3E: Preparation of benzyl 4-methylpiperidin-4-ylcarbamate: tert-Butyl 4-(benzyloxycarbonylamino)-4-methylpiperidine-1-carboxylate (2.38 g, 6.83 mmol) was dissolved in MeOH (10 mL) and treated with 4 M hydrogen chloride in dioxane (25.6 ml, 102 mmol). The solution was stirred at ambient temperature for 14 hours then concentrated in vacuo. The residue was dissolved in methylene chloride and adjusted to pH 10 with 15% NaOH. The layers were separated and the aqueous was washed with methylene chloride. The combined organic layers were dried over Na₂SO₄ and concentrated in vacuo. The residue was dissolved in methylene chloride (20 mL) and applied to a pre-equilibrated (methylene chloride) Varian Bond Elut SCX column (10 g). The column was eluted sequentially under slightly reduced pressure with 150 mL fractions of methylene chloride, 10% MeOH in methylene chloride, 20% (6% NH₄OH in MeOH) in methylene chloride. The final fraction was concentrated in vacuo then dissolved in methylene chloride, dried over Na₂SO₄ and concentrated in vacuo to provide the desired product (1.54 g). NMR (400 MHz, CDCl₃) 7.42-7.29 (m, 5H), 5.06 (s, 2H), 4.67-4.58 (brd s, 1H), 2.85-2.79 (m, 4H), 1.94-1.89 (brd m, 2H), 1.61-1.51 (m, 2H), 1.38 (s, 3H). MS APCI (+) m/z 249.0 (M+1) detected.

Step 4A: Preparation of benzyl 1-(6-fluoro-2-(7-(2-methoxyethoxy)imidazo[1,2-a]pyridin-3-yl)quinolin-8-yl)-4-methylpiperidin-4-ylcarbamate: Benzyl 4-methylpiperidin-4-ylcarbamate (0.058 g, 0.23 mmol), 8-bromo-6-fluoro-2-(7-(2-methoxyethoxy)imidazo[1,2-a]pyridin-3-yl)quinoline (0.075 g, 0.180 mmol), micronized Cs₂CO₃ (0.082 g, 0.25 mmol), Binap-racemic (0.022 g, 0.036 mmol) and Pd₂ dba₃ (0.016 g, 0.018 mmol) were combined in toluene (1 mL). The solution was degassed with argon then heated to reflux under argon for 14 hours. The reaction was cooled, loaded onto a silica gel column with CHCl₃ and purified by chromatography using a gradient from 1-20% (6% NH₄OH in MeOH)/ethyl acetate, (6.9 mg). MS APCI (+) m/z 584.2 (M+1) detected.

Step 4B: Preparation of 1-(6-fluoro-2-(7-(2-methoxyethoxy)imidazo[1,2-a]pyridin-3-yl)quinolin-8-yl)-4-methylpiperidin-4-amine: Benzyl 1-(6-fluoro-2-(7-(2-methoxyethoxy)imidazo[1,2-a]pyridin-3-yl)quinolin-8-yl)-4-methylpiperidin-4-ylcarbamate (0.069 g, 0.12 mmol) and Pearlman's catalyst (20% Pd(OH)₂ on carbon) (0.0042 g, 0.030 mmol) were dissolved in THF (0.5 mL), 95% EtOH (0.5 mL) and concentrated HCl (1 drop). The mixture was placed under hydrogen atmosphere (balloon pressure) and stirred at ambient temperature for 24 hours. The reaction mixture was filtered (nylon membrane, 0.45 μM) and concentrated in vacuo. The residue was purified by preparative TLC eluting with 10% (6% NH₄OH in MeOH) in chloroform to provide the desired product (2.6 mg). MS APCI (+) m/z 450.1 (M+1) detected.

### Example 65

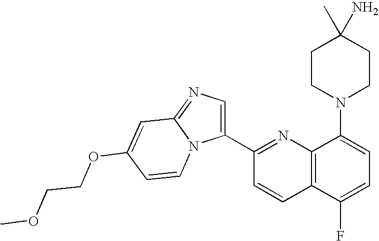

### 1-(5-fluoro-2-(7-(2-methoxyethoxy)imidazo[1,2-a]pyridin-3-yl)quinolin-8-yl)-4-methylpiperidin-4-amine

Step 1A: Preparation of 8-bromo-5-fluoro-2-methylquinoline: 2-Bromo-5-fluorobenzenamine (15 g, 78.9 mmol) was weighed into a 100 mL flask and dissolved in 100 mL of 6 N HCl. The reaction mixture was heated to reflux, followed by dropwise addition of (E)-but-2-enal (6.87 ml, 83 mmol) mixed with 1.0 mL deionized water over 25 minutes. Following complete addition, the reaction was heated at 100° C. for an additional 35 minutes. The reaction was cooled to ambient temperature, followed by addition of 50 mL of Et₂O. The reaction was stirred for 5 minutes, followed by removal of Et₂O by separatory funnel. ZnCl₂ (3.587 g, 26 mmol) was added to the aqueous layer in two portions and the reaction mixture was cooled to 0° C. over 30 minutes. The aqueous layer was adjusted to pH 8.0 using concentrated NH₄OH. The aqueous layer was extracted with Et₂O and EtOAc. The combined organic phases were dried over Na₂SO₄, filtered and concentrated in vacuo, affording the desired product (18.1 g) as a solid.

Step 1B: Preparation of 8-bromo-2-(dibromomethyl)-5-fluoroquinoline: 8-Bromo-5-fluoro-2-methylquinoline (18.1 g, 75.4 mmol) was weighed into a flask, followed by addition of NaOAc (37.1 g, 452 mmol). The solids were suspended in 500 mL of AcOH, and the reaction heated to 70° C. Bromine (11.6 ml, 226 mmol) was added dropwise over 25 minutes as a solution in 50 mL of AcOH. Following complete addition, the reaction was stirred at 100° C. for 1 hour. The reaction was then cooled to ambient temperature, then poured onto 700 cc of ice. The ice was allowed to melt completely and the mixture was extracted with ethyl acetate. The combined organic phases were dried over Na₂SO₄, filtered, concentrated in vacuo and dried under vacuum, affording the desired product (27 g, 90%).

Step 1C: Preparation of 8-bromo-5-fluoroquinoline-2-carboxylate and 8-bromo-5-fluoroquinoline-2-carboxylic acid: 8-Bromo-2-(dibromomethyl)-5-fluoroquinoline (25 g, 63 mmol) was weighed into a 1 flask and dissolved in 250 mL of EtOH, followed by addition of silver nitrate (34 g, 201 mmol) in 100 mL of 1:1 EtOH/H₂O. The reaction was heated to reflux for 1 hour, then filtered hot through a medium frit sintered glass funnel, affording 2.17 g of a powder. The mother liquor was concentrated in vacuo, followed by extractive work-up (500 mL EtOAc/water). The combined organic phases were dried over Na₂SO₄ and concentrated in vacuo to afford ethyl 8-bromo-5-fluoroquinoline-2-carboxylate (9.3 g, 99% yield) and 8-bromo-5-fluoroquinoline-2-carboxylic acid (8 g, 94% yield).

Step 1D: Preparation of (8-bromo-5-fluoroquinolin-2-yl)methanol: Ethyl 8-bromo-5-fluoroquinoline-2-carboxylate (5.52 g, 18.5 mmol) was weighed into 1000 mL flask and dissolved in 400 mL of DCM. The reaction was cooled to −78° C., followed by dropwise addition of DIBAL-H (49.4 ml, 74.1 mmol) over 10 minutes. The reaction was allowed to stir and warm to ambient temperature over 2 hours. The reaction was quenched with 10 mL MeOH and 100 mL of 1 N Rochelle's salt, followed by stirring overnight. The aqueous layer was extracted with EtOAc, followed by concentration in vacuo. The residue was purified by flash column chromatography (20-50% EtOAc/Hex), affording the desired product as a solid (2.25 g). MS APCI (+) m/z 256.1 (M+1) detected.

Step 1E: Preparation of 8-bromo-5-fluoroquinoline-2-carbaldehyde: (8-Bromo-5-fluoroquinolin-2-yl)methanol (1.85 g, 7.22 mmol), DMSO (8.20 ml, 116 mmol) and triethylamine (4.53 ml, 32.5 mmol) were weighed into a flask and dissolved in a 1:1 mixture of DCM/DMSO, followed by cooling to 0° C. Pyridine sulfur trioxide (4.02 g, 25.3 mmol) was added and the reaction was stirred at 0° C. for 1 hour. The reaction was poured into 50 mL water and extracted with EtOAc. The combined organic phases were dried over MgSO₄, filtered, and concentrated in vacuo affording a semi-solid, which was further purified by flash column chromatography 10-40% EtOAc/Hexane, affording 8-bromo-5-fluoroquinoline-2-carbaldehyde (1.71 g).

Step 1F: Preparation of 8-bromo-5-fluoro-2-(2-methoxyvinyl)quinoline: (Methoxymethyl)triphenylphosphonium chloride (1.9 g, 5.6 mmol) was weighed into a flask and dissolved in 40 mL of anhydrous THF. The reaction was cooled to 0° C., followed by dropwise addition of KOtBu (6.1 ml, 6.1 mmol). The reaction was stirred for 15 minutes at ambient temperature, followed by dropwise addition of 8-bromo-5-fluoroquinoline-2-carbaldehyde (1.3 g, 5.1 mmol) as a solution in 10 mL of THF over 3 minutes, affording an immediate dark red/brown color change. The reaction was allowed to stir at ambient temperature for 12 hours. The reaction was concentrated in vacuo, followed by trituration with Et₂O, then ethyl acetate, to afford the crude desired product, which was used in the next step without purification.

Step 2A: Preparation of 2-chloro-4-(2-methoxyethoxy)pyridine: A mixture of 2-chloro-4-nitropyridine (43.6 g, 275 mmol) and 2-methoxyethanol (325 ml, 425 mmol) was cooled to 0° C. Potassium 2-methylpropan-2-olate (35.7 g, 302 mmol) was added and the resulting mixture was stirred while warming to ambient temp over 2 hours. The reaction mixture was concentrated under reduced pressure followed by dilution with 500 ml of water. The resulting mixture was extracted with dichloromethane. The combined organic layers were dried over MgSO₄ and concentrated under reduced pressure to produce the desired compound as an oil. (50.2 g) MS APCI (+) m/z 188 and 189.9 (M+1 of each isotope) detected.

Step 2B: Preparation of 4-(2-methoxyethoxy)pyridin-2-amine: A steady stream of nitrogen was passed through a mixture of 2-chloro-4-(2-methoxyethoxy)pyridine (50.1 g, 267 mmol), Pd₂ dba₃ (4.89 g, 5.34 mmol), XPHOS (5.09 g, 10.7 mmol) and tetrahydrofuran (445 ml) for 10 minutes. To the resulting degassed mixture was added lithium bis(trimethylsilyl)amide (561 ml, 561 mmol). After addition, the resulting mixture was heated to 60° C. for 18 hours. The reaction was cooled to ambient temperature and diluted with 1 N hydrochloric acid (200 mL). The resulting solution was washed with methyl-tert-butyl ether. The pH of the aqueous layer was adjusted to 11 with 6 N NaOH and extracted with dichloromethane. The combined organic layers were dried over MgSO₄ and concentrated under reduced pressure to yield title compound (35 g) MS APCI (+) m/z 169 (M+1) detected.

Step 2C: Preparation of 8-bromo-5-fluoro-2-(7-(2-methoxyethoxy)-imidazo[1,2-a]pyridin-3-yl)quinoline: 8-Bromo-5-fluoro-2-(2-methoxyvinyl)quinoline (2.4 g, 8.5 mmol) was dissolved in a solution of 20 mL of THF and 4 mL of deionized water. N-Bromosuccinimide (1.59 g, 8.9 mmol) was added and the reaction was stirred for 2 hours. 4-(2-methoxyethoxy)pyridin-2-amine (1.43 g, 8.51 mmol) was added, and the reaction was heated to reflux for 10 hours. The crude reaction mixture was concentrated in vacuo affording a solid which was triturated successively with EtOAc and Et₂O, followed by trituration with a mixture of Et₂O and CH₂Cl₂ to afford the desired product (746 mg) as a solid. MS APCI (+) m/z 416.2 (M+1) detected.

Step 3A: Preparation of 1-tert-butyl 4-ethyl piperidine-1,4-dicarboxylate: The compound was prepared following a procedure outlined in PCT Publication No. WO 01/40217. Ethyl piperidine-4-carboxylate (8.639 ml, 56.10 mmol) was dissolved in dichloromethane (55 mL) and treated in three equal portions with di-tert-butyl dicarbonate (12.24 g, 56.10 mmol). Each addition caused vigorous bubbling and a bit of temperature rise. After the additions, the solution was stirred at ambient temperature for 14 hours. The solution was extracted with saturated NaHCO₃, dried over Na₂SO₄ and concentrated in vacuo to provide the desired product as an oil (14.1 g).

Step 3B: Preparation of 1-tert-butyl 4-ethyl 4-methylpiperidine-1,4-dicarboxylate: The compound was prepared following a procedure outlined in PCT Publication No. WO 01/40217. 1-tert-Butyl 4-ethyl piperidine-1,4-dicarboxylate (7.12 g, 27.7 mmol) was dissolved in THF (30 mL) and cooled to −40° C. LHMDS (55.3 ml, 55.3 mmol) was added slowly and the solution was stirred at −40° C. for 1 hour. Iodomethane (3.45 ml, 55.3 mmol) was added and the reaction mixture was warmed to ambient temperature and stirred for 14 hours. The reaction was quenched with water and saturated NaHCO₃. After diluting with methylene chloride, the layers were separated. The aqueous layer was washed with methylene chloride, and the combined organic layers were washed with saturated NaCl, dried over Na₂SO₄ and concentrated in vacuo to provide the desired product as an oil (quantitative).

Step 3C: Preparation of 1-(tert-butoxycarbonyl)-4-methylpiperidine-4-carboxylic acid: The compound was prepared following a procedure outlined in PCT Publication No. WO 01/40217. 1-tert-Butyl 4-methylpiperidine-1,4-dicarboxylate (54.2 g, 200 mmol) was dissolved in a solution of EtOH (400 mL) and 2 N NaOH (200 mL). The mixture was heated to 60° C. for 60 hours, then cooled and concentrated in vacuo. The solution was extracted with Et₂O, and the aqueous layer was adjusted to pH 3 with a mixture of concentrated HCl followed by 3 N HCl. The aqueous was extracted with ethyl acetate, then the combined organic layers were washed with saturated NaCl, dried over Na₂SO₄ and concentrated in vacuo to provide the desired product as a solid (45.1 g).

Step 3D: Preparation of tert-butyl 4-(benzyloxycarbonylamino)-4-methylpiperidine-1-carboxylate: The compound was prepared following a procedure outlined in Madar, D. J.; et al.; J. Med. Chem. 2006, 49, 6416-6420, and supplementary materials. 1-(tert-Butoxycarbonyl)-4-methylpiperidine-4-carboxylic acid (5.00 g, 20.5 mmol) was dissolved in toluene (40 mL) was treated at ambient temperature with triethylamine (4.30 ml, 30.8 mmol) and diphenylphosphoryl azide (5.98 ml, 27.7 mmol). The reaction was stirred at ambient temperature for 45 minutes, and phenylmethanol (10.6 ml, 102 mmol) was added and the mixture was heated to 80° C. for 16 hours. The reaction mixture was concentrated in vacuo. The residue was redissolved in ethyl acetate and washed with saturated NH₄Cl and saturated NaCl. The organic layer was dried over Na₂SO₄ and concentrated in vacuo to provide the desired product as a semi-solid (25 g), which was utilized in the next step without purification.

Step 3E: Preparation of benzyl 4-methylpiperidin-4-ylcarbamate: tert-Butyl 4-(benzyloxycarbonylamino)-4-methylpiperidine-1-carboxylate (2.38 g, 6.83 mmol) was dissolved in MeOH (10 mL) and treated with 4 M hydrogen chloride in dioxane (25.6 ml, 102 mmol). The solution was stirred at ambient temperature for 14 hours then concentrated in vacuo. The residue was dissolved in methylene chloride and adjusted to pH 10 with 15% NaOH. The layers were separated and the aqueous was washed with methylene chloride. The combined organic layers were dried over Na₂SO₄ and concentrated in vacuo. The residue was dissolved in methylene chloride (20 mL) and applied to a pre-equilibrated (methylene chloride) Varian Bond Elut SCX column (10 g). The column was eluted sequentially under slightly reduced pressure with 150 mL fractions of methylene chloride, 10% MeOH in methylene chloride, 20% (6% NH₄OH in MeOH) in methylene chloride. The final fraction was concentrated in vacuo then redissolved in methylene chloride, dried over Na₂SO₄ and concentrated in vacuo to provide the desired product (1.54 g). ¹H NMR (400 MHz, CDCl₃) δ 7.42-7.29 (m, 5H), 5.06 (s, 2H), 4.67-4.58 (brd s, 1H), 2.85-2.79 (m, 4H), 1.94-1.89 (brd m, 2H), 1.61-1.51 (m, 2H), 1.38 (s, 3H). MS APCI (+) m/z 249.0 (M+1) detected.

Step 4A: Preparation of benzyl 1-(5-fluoro-2-(7-(2-methoxyethoxy)imidazo[1,2-a]pyridin-3-yl)quinolin-8-yl)-4-methylpiperidin-4-ylcarbamate: Benzyl 4-methylpiperidin-4-ylcarbamate (0.12 g, 0.49 mmol), 8-bromo-5-fluoro-2-(7-(2-methoxyethoxy)imidazo[1,2-a]pyridin-3-yl)quinoline (0.14 g, 0.33 mmol), micronized Cs₂CO₃ (0.15 g, 0.46 mmol), Binap-racemic (0.041 g, 0.066 mmol) and Pd₂ dba₃ (0.030 g, 0.033 mmol) were combined in toluene (2 mL). The solution was degassed with argon and heated to reflux under argon for 14 hours. The reaction was cooled briefly and additional benzyl 4-methylpiperidin-4-ylcarbamate (0.12 g, 0.49 mmol), Binap-racemic (0.041 g, 0.066 mmol), and Pd₂ dba₃ (0.030 g, 0.033 mmol) were added and after re-equilibrating with argon gas, heating continued for an additional 26 hours. MS APCI (+) m/z 584.3 (M+1) detected. After 40 hours, the reaction is cooled and filtered through a nylon membrane. The filtrate is concentrated in vacuo and purified by chromatography on silica gel, eluting with a gradient from 1-20% (6% NH₄OH in MeOH)/thyl acetate.

Step 4B: Preparation of 1-(5-fluoro-2-(7-(2-methoxyethoxy)imidazo[1,2-a]pyridin-3-yl)quinolin-8-yl)-4-methylpiperidin-4-amine: Benzyl 1-(5-fluoro-2-(7-(2-methoxyethoxy)imidazo[1,2-a]pyridin-3-yl)quinolin-8-yl)-4-methylpiperidin-4-ylcarbamate is dissolved in a mixture of MeOH and ethyl acetate (1:1, 0.2 M) and treated with 10% Pd on carbon (0.1 eq.). The mixture is subjected to an atmosphere of hydrogen gas (balloon pressure) and stirred at ambient temperature for 24-48 hours. The catalyst is removed by filtration and the residue is concentrated in vacuo then purified by chromatography on silica gel, eluting with a gradient from 1-20% (6% NH₄OH in MeOH)/methylene chloride.

### Example 66

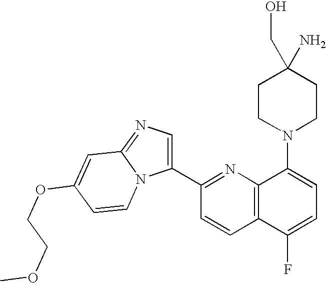

### (4-amino-1-(5-fluoro-2-(7-(2-methoxyethoxy)imidazo[1,2-a]pyridin-3-yl)quinolin-8-yl)piperidin-4-yl)methanol

Step A. Preparation of (4-amino-1-(5-fluoro-2-(7-(2-methoxyethoxy)imidazo[1,2-a]pyridin-3-yl)quinolin-8-yl)piperidin-4-yl)methanol: The compound can be prepared according to example 61, using benzyl 4-(hydroxymethyl)piperidin-4-ylcarbamate benzyl 4-(hydroxymethyl)piperidin-4-ylcarbamate in place of benzyl trans-3-fluoropiperidin-4-ylcarbamate.

Step B. Preparation of (4-amino-1-(5-fluoro-2-(7-(2-methoxyethoxy)imidazo[1,2-a]pyridin-3-yl)quinolin-8-yl)piperidin-4-yl)methanol: The compound can be prepared according to Example 48 using (4-amino-1-(5-fluoro-2-(7-(2-methoxyethoxy)imidazo[1,2-a]pyridin-3-yl)quinolin-8-yl)piperidin-4-yl)methanol in place of (4-Amino-1-(2-(7-(2-methoxyethoxy)imidazo[1,2-a]pyridin-3-yl)quinolin-8-yl)piperidin-4-yl)methanol.

### Example 67

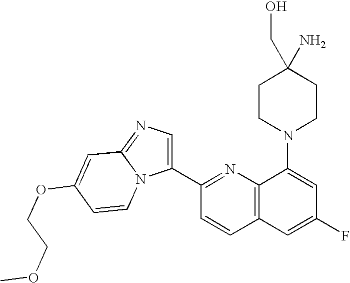

### (4-amino-1-(6-fluoro-2-(7-(2-methoxyethoxy)imidazo[1,2-a]pyridine-3-yl)quinolin-8-yl)piperidin-4-yl)methanol

Step A. Preparation of methyl 4-(benzyloxycarbonylamino)-1-(6-fluoro-2-(7-(2-methoxyethoxy)imidazo[1,2-a]pyridin-3-yl)quinolin-8-yl)piperidine-4-carboxylate: The compound can be prepared according to example 26, using benzyl 4-(hydroxymethyl)piperidin-4-ylcarbamate in place of tert-butyl piperidin-4-ylcarbamate.

Step B. Preparation of methyl 4-amino-1-(6-fluoro-2-(7-(2-methoxyethoxy)imidazo[1,2-a]pyridin-3-yl)quinolin-8-yl)piperidine-4-carboxylate: The Cbz group can be removed according the conditions of Example 27, step E.

Step C. Preparation of (4-amino-1-(6-fluoro-2-(7-(2-methoxyethoxy)imidazo[1,2-a]pyridin-3-yl)quinolin-8-yl)piperidin-4-yl)methanol: The compound can be prepared according the conditions of Example 48, using methyl 4-amino-1-(6-fluoro-2-(7-(2-methoxyethoxy)imidazo[1,2-a]pyridin-3-yl)quinolin-8-yl)piperidine-4-carboxylate in place of (4-amino-1-(2-(7-(2-methoxyethoxy)imidazo[1,2-a]pyridin-3-yl)quinolin-8-yl)piperidin-4-yl)methanol.

### Example 68

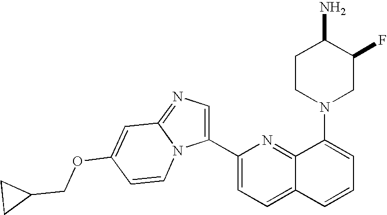

### (cis)-1-(2-(7-(cyclopropylmethoxy)imidazo[1,2-a]pyridin-3-yl)quinolin-8-yl)-3-fluoropiperidin-4-amine dihydrochloride

Step A. Preparation of 8-bromo-2-(7-(cyclopropylmethoxy)imidazo[1,2-a]pyridin-3-yl)quinoline: The compound was prepared according the procedure of example 1, using cyclopropylmethanol in place of 2-methoxyethanol. MS APCI (+) m/z 394/396 (Br isotope) (M+1) detected.

Step B. Preparation of (cis)-1-(2-(7-(cyclopropylmethoxy)imidazo[1,2-a]pyridin-3-yl)quinolin-8-yl)-3-fluoropiperidin-4-amine: The compound was prepared according to the procedures used for Example 27, using 8-bromo-2-(7-(cyclopropylmethoxy)imidazo[1,2-a]pyridin-3-yl)quinoline in place of 2-(7-(2-methoxyethoxy)imidazo[1,2-a]pyridin-3-yl)quinolin-8-yl trifluoromethanesulfonate. The free base was then converted to the dihydrochloride salt using standard conditions. MS APCI (+) m/z 432 (M+1) detected.

### Example 69

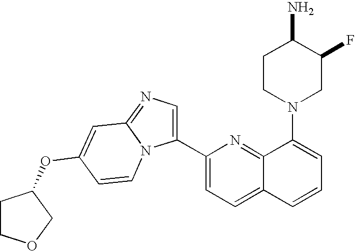

### cis-3-Fluoro-1-(2-(7-((S)-tetrahydrofuran-3-yloxy)imidazo[1,2-a]pyridin-3-yl)quinolin-8-yl)piperidin-4-amine

Step A. Preparation of (S)-8-bromo-2-(7-(tetrahydrofuran-3-yloxy)imidazo[1,2-a]pyridin-3-yl)quinoline: The compound was prepared according the procedure of example 1, using (S)-tetrahydrofuran-3-ol in place of 2-methoxyethanol. MS APCI (+) m/z 410/412 (Br isotope) (M+1) detected.

Step B. Preparation of cis-3-fluoro-1-(2-(7-((S)-tetrahydrofuran-3-yloxy)imidazo[1,2-a]pyridin-3-yl)quinolin-8-yl)piperidin-4-amine: The compound was prepared according to the procedures of Example 27, using (S)-8-bromo-2-(7-(tetrahydrofuran-3-yloxy)imidazo[1,2-a]pyridin-3-yl)quinoline in place of 2-(7-(2-methoxyethoxy)imidazo[1,2-a]pyridin-3-yl)quinolin-8-yl trifluoromethanesulfonate. Isolated as a 1:1 mixture of diastereomers. MS APCI (+) m/z 448 (M+1) detected.

### Example 70

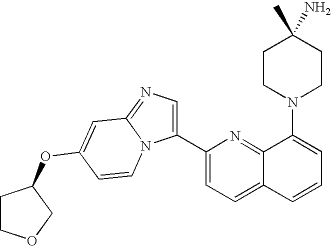

### (R)-4-Methyl-1-(2-(7-(tetrahydrofuran-3-yloxy)imidazo[1,2-a]pyridin-3-yl)quinolin-8-yl)piperidin-4-amine

Step A. Preparation of (R)-8-bromo-2-(7-(tetrahydrofuran-3-yloxy)imidazo[1,2-a]pyridin-3-yl)quinoline: The compound was prepared according to the procedures used for Example 1, using (R)-tetrahydrofuran-3-ol in place of 2-methoxyethanol. MS APCI (+) m/z 410/412 (Br isotope) (M+1) detected

Step B. Preparation of (R)-4-methyl-1-(2-(7-(tetrahydrofuran-3-yloxy)imidazo[1,2-a]pyridin-3-yl)quinolin-8-yl)piperidin-4-amine: The compound was prepared according to the procedures used for Example 30, using (R)-8-bromo-2-(7-(tetrahydrofuran-3-yloxy)imidazo[1,2-a]pyridin-3-yl)quinoline in place of 2-(7-(2-methoxyethoxy)imidazo[1,2-a]pyridin-3-yl)quinolin-8-yl trifluoromethanesulfonate. MS APCI (+) m/z 432 (M+1) detected.

### Example 71

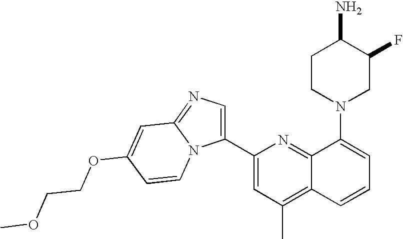

### (cis)-3-fluoro-1-(2-(7-(2-methoxyethoxy)imidazo[1,2-a]pyridin-3-yl)-4-methylquinolin-8-yl)piperidin-4-amine

The compound was prepared according to the procedure used for Example 31, using benzyl (cis)-3-fluoropiperidin-4-ylcarbamate in place of tert-butyl piperidin-4-ylcarbamate. The Cbz group was removed according to the conditions used in Example 27, Step E, to give the title compound. MS APCI (+) m/z 450 (M+1) detected.

### Example 72

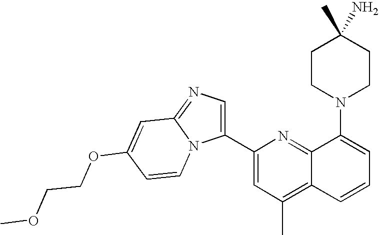

### 1-(2-(7-(2-methoxyethoxy)imidazo[1,2-a]pyridin-3-yl)-4-methylquinolin-8-yl)-4-methylpiperidin-4-amine

The protected amino compound can be prepared according to the procedures used for Example 31, using benzyl 4-methylpiperidin-4-ylcarbamate in place of tert-butyl piperidin-4-ylcarbamate. The Cbz group can be removed according to the procedure used for Example 27, Step E, to give the title compound.

1. A compound of general Formula I: 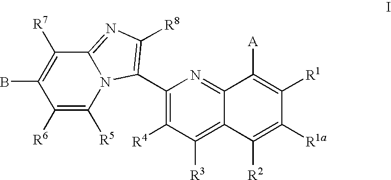
or a pharmaceutically acceptable salt thereof, wherein:
A is a 5-8 membered N-linked heterocyclic ring having at least one nitrogen atom and optionally substituted with one or more R⁹ groups;
B is H, CN, OR^{h}, Ar¹, hetAr², C(O)NRⁱR^{j}, C(O)-hetCyc³, CO₂(1-6C alkyl), C(O)NH(1-6C alkyl)-hetCyc³, C(O)(1-6C alkyl)-hetCyc³, SR^{k}, SO₂N(1-6C alkyl)₂, or (1-6C alkyl)NR′R″;
R¹, R², R³ and R⁴ are independently H, F, Cl, CN, Me, Et, isopropyl, cyclopropyl, C(O)NR′R″, CH₂OH, or hetAr³;
R^{1a} is H, F, Cl, CN, Me, or CF₃;
R⁵, R⁶, R⁷ and R⁸ are independently H, F, Cl, CN or Me;
each R⁹ is independently selected from halogen, CN, CF₃, (1-6C)alkyl, NR^{a}R^{b}, -(1-6C alkyl)NR^{a}R^{c}, OR^{a}, (1-6C alkyl)OR^{a} [optionally substituted with amino], C(O)NR^{a}R^{c}, C(O)(CR^{x}R^{y})NR^{a}R^{c}, NHC(O)R^{e}, NHC(O)(CR^{m}Rⁿ)NR^{a}R^{c}, NHC(O)NR^{f}R^{g}, (1-6C alkyl)-hetAr¹, (1-6C alkyl)-hetCyc¹, oxo and CO₂(1-6C alkyl);
each R^{a} is independently H or (1-6C)alkyl;
each R^{b} is independently H, (1-6C)alkyl, (1-6C alkyl)OH, (3-6C)cycloalkyl, CH₂hetAr⁴, (1-6C fluoroalkyl) or -(1-6C alkyl)-O-(1-6C alkyl),
or NR^{a}R^{b} forms a 4-6 membered heterocyclic ring optionally substituted with OH;
each R^{c} is independently H, (1-6C)alkyl, (3-6C)cycloalkyl, or aryl;
each R^{e} is independently (1-6C alkyl);
each R^{f} and R^{g} is independently H or (1-6C alkyl);
R^{h} is H, CF₃, (1-6C)alkyl, (1-6Calkyl)-(3-6C cycloalkyl), (1-6C alkyl)-O-(1-6C alkyl), (1-6C alkyl)OH, (1-6C alkyl)-S-(1-6C alkyl), (1-6C alkyl)NR′R″, hetCyc⁴, (1-6C alkyl)hetCyc⁴, (1-6C alkyl)aryl, or (1-6C alkyl)-hetAr⁵;
Rⁱ is H or 1-6C alkyl;
R^{j} is (1-6C)alkyl, (1-6C alkyl)-O-(1-6C alkyl), or (1-6C alkyl)-OH;
R^{k} is (1-6C)alkyl, (3-6C)cycloalkyl, or (1-6C alkyl)-O-(1-6C alkyl);
R^{m} and Rⁿ are independently H or (1-6C alkyl);
R^{x} and R^{y} are independently H or (1-6C alkyl),
or R^{x} and R^{y} together with the atom to which they are attached form a cyclopropyl ring;
Ar¹ is aryl optionally substituted with OH, O-(1-6C alkyl), C(O)₂(1-6C alkyl), or (1-6C alkyl)NR′R″;
hetCyc¹ is a 5-6 membered heterocyclic ring which is optionally substituted with (1-6C)alkyl or OH;
hetCyc³ and hetCyc⁴ are independently a 5 or 6 membered heterocyclic ring optionally substituted with OH or —O(1-6C alkyl);
hetAr¹ and hetAr² are independently a 5-6 membered heteroaryl ring optionally substituted with one to three groups independently selected from (1-6C)alkyl, (3-6C)cycloalkyl, halogen, CN, CF₃, OCH₂F, OCF₃, O(1-6C alkyl), O(3-6C)cycloalkyl, and NR′R″;
hetAr³ and hetAr⁴ are independently a 5-6 membered heteroaryl ring;
hetAr⁵ is a 5-6 membered heteroaryl ring optionally substituted with (1-6C)alkyl; and
R′ and R″ are independently H or (1-6C)alkyl.

2. A compound of claim 1, wherein A is optionally substituted with one or more R⁹ groups independently selected from (1-6C)alkyl, NR^{a}R^{b}, OR^{a}, (1-6C alkyl)OR^{a}, C(O)NR^{a}R^{c}, -(1-6C alkyl)NR^{a}R^{c}, halogen, CO₂(1-6C alkyl), and CF₃.

3. A compound as defined in claim 1, wherein A is optionally substituted with one or more R⁹ groups independently selected from methyl, NH₂, NMe₂, —NHCH(CH₃)CH₂F, NHCH₂CH₂OCH₃, —NHCH₂CH₂OH, N(CH₃)CH₂CH₂OH, 1-azetidin-3-ol, OH, CH₂OH, C(O)NHMe, CH₂NH₂, CH₂CH₂NMe₂, F, CO₂Me and CF₃.

4. A compound as defined in claim 1, wherein A is optionally substituted with one or more R⁹ groups independently selected from methyl, NH₂, NHCH(CH₃)CH₂F, OH, CH₂OH, and F.

5. A compound as defined in claim 1, wherein A is optionally substituted with one or more R⁹ groups independently selected from F, NH₂, and CH₂OH.

6. A compound as defined in claim 1, wherein A is an optionally substituted piperidinyl, piperazinyl or pyrrolidinyl ring.

7. A compound as defined in claim 1, wherein B is selected from H, CN, OR^{h}, hetAr², C(O)NRⁱR^{j}, and CO₂(1-6 C alkyl).

8. A compound of claim 7, wherein B is selected from H, CN, —O(1-6C alkyl)-O-(1-6C alkyl), —O(1-6C alkyl)OH, —O(1-6Calkyl)-(3-6C cycloalkyl), —O(1-6C alkyl)NR′R″, a pyridyl ring, a pyrimidyl ring, C(O)N(1-6C alkyl)₂, and CO₂(1-6 C alkyl).

9. A compound of claim 8, wherein B is selected from H, CN, —OCH₂CH₂OMe, —OCH₂CH₂OH, —OCH₂(cyclopropyl), 2-pyridyl, 3-pyridyl, 2-pyrimidyl, —OCH₂CH₂NH₂, C(O)NMe₂, and C(O)₂Me.

10. A compound as defined in claim 1, wherein B is OR^{h} or hetAr².

11. A compound of claim 10, wherein B is selected from —O(1-6C alkyl)-O-(1-6C alkyl), O(1-6Calkyl)-(3-6C cycloalkyl), —O(1-6C alkyl)OH, a pyridyl ring, and a pyrimidyl ring.

12. A compound of claim 11, wherein B selected from —OCH₂CH₂OMe, —OCH₂CH₂OH, —OCH₂(cyclopropyl), 2-pyridyl, 3-pyridyl, and 2-pyrimidyl.

13. A compound as defined in claim 1, wherein B is OR^{h}.

14. A compound of claim 13, wherein B is selected from —O(1-6C alkyl)-O-(1-6C alkyl), O(1-6Calkyl)-(3-6C cycloalkyl), —O(1-6C alkyl)OH.

15. A compound of claim 14, wherein B is —OCH₂CH₂OMe.

16. A compound as defined in claim 1, wherein R^{1a} is H, F or CF₃.

17. A compound as defined in claim 1, wherein R² is H or F.

18. A compound as defined in claim 1, wherein R³ is H, methyl or oxazolyl.

19. A compound as defined in claim 1, wherein each of R⁵, R⁶, R⁷ and R⁸ is hydrogen.

20. A compound as defined in claim 1, wherein each of R¹ and R⁴ is hydrogen.

21. A pharmaceutical composition, which comprises a compound of Formula I as defined in claim 1, or a pharmaceutically acceptable salt thereof, and a pharmaceutically acceptable diluent or carrier.

22. (canceled)

23. (canceled)

24. A method of treating a class 3 and/or class 5 receptor tyrosine kinase-mediated condition in a mammal, which comprises administering to said mammal a therapeutically effective amount of a compound of Formula I as defined in claim 1, or a pharmaceutically acceptable salt thereof.

25. The method of claim 24, wherein said condition is cancer.

26. The method of claim 25, wherein said condition is fibrosis.

27. A process for the preparation a compound of claim 1, which comprises:
(a) coupling a corresponding compound having the formula II 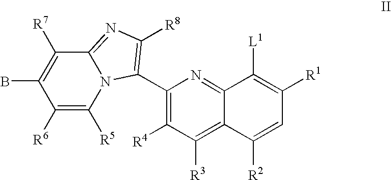
wherein L¹ represents a leaving atom or group, with a compound having the formula HNR¹⁰R¹¹ wherein NR¹⁰R¹¹ forms a 5-8 membered heterocyclic ring optionally substituted with one or more R⁹ groups, using a palladium catalyst and a ligand in the presence of a base; or
(b) for a compound of Formula I where B is OR^{h}, reacting a corresponding compound having the Formula III 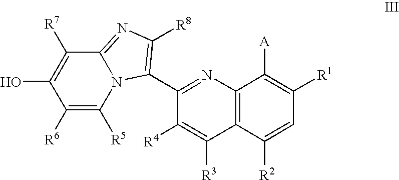
with a compound of the formula R^{h}-L² wherein L² represents a leaving atom or group in the presence of a base; or
(c) for a compound of Formula I where B is OR^{h}, reacting a corresponding compound having the Formula III with a compound having the formula R^{h}—OH in the presence of a coupling reagent; or
(d) for a compound having the Formula I wherein A is: 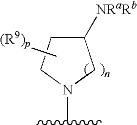
wherein n is 1-3, p is 0-4, R^{b} is other than hydrogen, and R^{a} is as defined in claim 1, reacting a corresponding compound having the formula IV 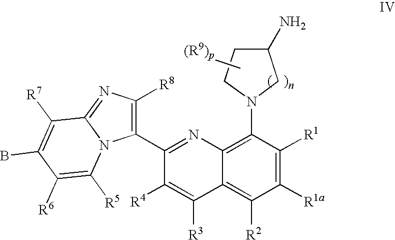
with a compound having the formula R^{a}C(═O)R^{b} where R^{b} is other than hydrogen, followed by treatment with a reducing agent; or
(e) for a compound of Formula I wherein A is: 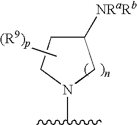
wherein n is 1-3 and p is 0-4, reacting a corresponding compound having the formula V 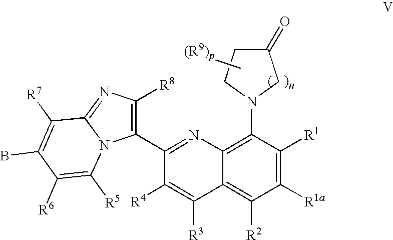
with a compound having the formula HNR^{a}R^{b} followed by treatment with a reducing agent; or
(f) for a compound of Formula I wherein the B group has the formula —O(1-6C alkyl)NH₂, reacting a corresponding compound having the formula VI 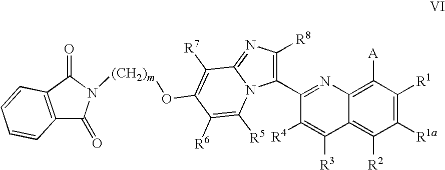
wherein m is an integer from 1-6, with a hydrazine;
(g) for a compound of Formula I wherein the B group has the formula —O(CH₂CH₂)OH, demethylating a corresponding compound having the formula 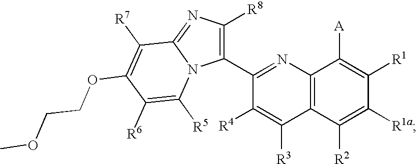
and
removing any protecting group or groups and, if desired, forming a salt.
